(19) United States
(12) Patent Application Publication
(10) Pub. No.: US 2026/0226057 A1
(43) Pub. Date: Aug. 6, 2026

(54) TRICYCLIC AZEPINONE DERIVATIVES AS SYSTEM XC INHIBITORS

(71) Applicant: UCB BIOPHARMA SRL, Brussels (BE)

(72) Inventors: Pierre BURSSENS, Brussels (BE); Jean KEYAERTS, Brussels (BE); Marie Adolphine C. LEDECQ, Brussels (BE); Baptiste MANTEAU, Brussels (BE); Laurent PROVINS, Brussels (BE); Dominique Louis L. SWINNEN, Brussels (BE)

(21) Appl. No.: 19/151,823

(22) PCT Filed: Jan. 29, 2024
(86) PCT No.: PCT/EP2024/052061

§ 371 Date: Jul. 29, 2025

(30) Foreign Application Priority Data
Feb. 1, 2023 (EP) 23154379.4

(51) Int. Cl. C07D 471/14; A61K 31/551; C07D 471/04; C07D 513/14

(52) U.S. Cl. CPC C07D 471/14; A61K 31/551; C07D 471/04; C07D 513/14

(57) ABSTRACT

The present invention relates to a compound of formula (I) or a pharmaceutically acceptable salt thereof, which is useful for the treatment of diseases and/or disorders in which System Xc- plays a role.

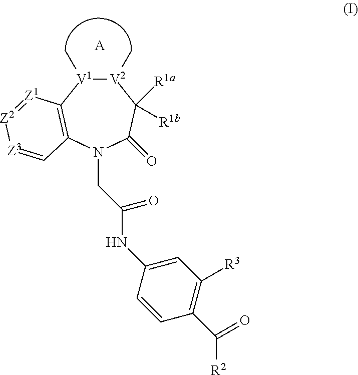

The invention relates to substituted 7-membered cyclic amides derivatives and their use in therapy.

In particular, the present invention relates to pharmacologically active substituted 7-membered cyclic amides or ureas derivatives and analogs thereof.

More particularly, the present invention relates to (cyclo)alkylcarbonyl substituted (hetero)aryl-acetamide of 7-membered cyclic amides derivatives and analogs thereof. The compounds according to the present invention modulate the System Xc⁻ cystine/glutamate antiporter and accordingly are of benefit as pharmaceutical agents for the treatment of diseases in which System Xc⁻ cystine/glutamate antiporter plays a role.

### BACKGROUND OF THE INVENTION

System Xc⁻, also known as the cystine/glutamate antiporter, is an amino acid transporter that mediates the extrusion of intracellular L-glutamate and the uptake of extracellular L-cystine, which undergoes intracellular reduction to L-cysteine. The influx of L-cystine serves as a rate-limiting step in providing L-cysteine, which is required for the synthesis of glutathione (GSH), the principal antioxidant in cells. L-Glutamate extruded by System Xc⁻ can serve as neurotransmitter. System Xc⁻ is a complex formed of two proteins, xCT (coded by the SLC7A11 gene) also called the light chain, and CD98 hc (SLC3A2) also called heavy chain or 4F2 hc. System Xc⁻ is expressed predominantly in the brain, in some glial cells such as astrocytes and microglia, and in non-CNS cells such as endothelial cells, fibroblasts, macrophages and hepatocytes.

In many different cancer types, system Xc⁻ is overexpressed compared to normal tissue. Those include, but are not limited to glioma (particularly glioblastoma) (Takeuchi et al. Neurosurgery (2013), 72, 33-41), colon carcinoma, colorectal carcinoma (Sugano et al. Anticancer Res (2015), 35, 677-682), non-small cell lung carcinoma (adenocarcinomas and squamous cell carcinomas) and other lung cancer types (Ji et al. Oncogene (2018), 37, 5007-5019), esophageal carcinoma, cancer stem cells in triple negative breast cancer (Conti et al., Cancer Immunol Res (2020), 8, 1039-105) and hepatocellular carcinoma (Kavanaugh et al., Mol Imaging Biol (2016) 18, 924-934).

High system Xc⁻ expression is associated with poor prognosis in several cancers including but not limited to colon carcinoma (Lim et al., Proc Natl Acad Sci USA (2019), 116, 9433-9442), adrenocortical carcinoma, kidney carcinoma (Wang et al., Oncotarget 2016, 7, 29901-29915), hepatocellular carcinoma (Kinoshita et al., Oncolumn Rep (2013), 29, 685-689), mesothelioma, lung carcinoma (Ji et al. Oncogene (2018), 37, 5007-5019), sarcoma, uveal melanoma and gastric cancer (Luo et al., Oncotarget 8, (2017), 112530-112549). In pancreatic ductal adenocarcinoma, a particular form of pancreatic carcinoma, stroma cells heavily rely on cysteine to prevent ferroptotic cell death and depletion of SLC7A11 in cancer-associated fibroblasts prevents orthotopic pancreatic tumor formation (Sharbeen et al., Cancer Res (2021); DOI: 10.1158/0008-5472.CAN-20-2496). In other cancers, System Xc⁻ plays a crucial role in tumorigenesis, because down regulation of SLC7A11 (the light chain of system Xc⁻) in cancer cells decreases cancer cell proliferation, tumor progression and invasion (Badgley et al., Science (2020), 368, 85-89; Ede et al., Haematologica (2018), 103, 1496-1501; Hu et al., J Clin Invest (2020), 130, 1752-1766; Lei et al., Cell Res (2020), 30, 146-162; Lin et al., Am J Cancer Res (2020), 10, 3106-3126).

High system Xc⁻ levels also confer to the cell increased capacity for the anti oxidant GSH synthesis, defense against reactive oxygen species (ROS) and tumor growth (Liu et al., Mol Ther (2020), 28, 2358-2366).

In addition, SLC7A11, cystine and cysteine have been described to play a role in radiotherapy resistance and in multidrug resistance in several cancer types (Horibe et al., Biochem Biophys Res Commun (2018), 507, 426-432; Koppula et al., Cell Res (2020), 30, 146-162).

Thus, inhibiting or blocking System Xc⁻ may be useful for the treatment of certain cancers where System Xc⁻ plays a role.

Blocking System Xc⁻ can also synergize with other therapies targeting tumor growth. For example, inhibition of System Xc⁻ preventing cancer stem cell metastasis, together with chemotherapy treatment blocking tumor growth (induced by oncogenes such as HER2, p53, Kras and others), leads to additional therapeutic effects in breast, esophageal and other cancer cell lines and models (Conti et al., Cancer Immunol Res (2020), 8, 1039-53; Liu et al., Nat Commun (2017), 8, 14844).

In several cancer cells, toxic lipid peroxidation induced by inhibition of System Xc, when combined to conventional cancer therapy can have a synergistic effect, lead to cancer cell death and overcome resistance to this conventional cancer therapy (Lin et al., Am J Cancer Res (2020), 10, 3106-3126; Zhu et al. Cancer Res (2021) 77(8), 2064-2077).

Therefore, molecules inhibiting system Xc⁻ could be used alone or in combination therapy, with molecules or treatments targeting other mechanisms and pathways involved in cancer biology, and thereby help overcome drug resistance in current cancer treatments or enhance the effect of certain existing treatments. Glutamate release due to upregulation of system Xc⁻ in cancer cells also affects tumorigenesis, and inhibition of glutamate release is correlated with a decrease in proliferation not only in brain tumors, but also in non-brain carcinoma ((Savaskan et al., Nature Medicine (2008), 14, 629; Lewerenz et al., Antioxid Redox Signal (2013), 18, 522-555; Corsi et al., Int J Mol Sci (2019), 20).

System Xc⁻ induced efflux of L-Glutamate into the extracellular space can contribute to excitatory signaling and to excitotoxicity, leading to seizures, neuronal death, and other brain pathologies through activation of postsynaptic glutamate receptors on neurons. Conversely, mice lacking system Xc⁻ have decreased brain glutamate receptors and demonstrate decreased or delayed epileptogenesis (Leclercq et al., Epilepsia (2019), 60, 1412-1423). Glioblastoma cells expressing elevated System Xc⁻ levels release high levels of glutamate, which activates glutamate receptors on neighboring neurons, and induces neuronal hyperactivity and seizures (Marcus et al., J. Neurooncol. (2010), 97, 11-23; Robert et al., (2015), Sci Transl Med 7, 289ra286).

Inhibiting system Xc⁻ function or expression could therefore prevent glutamate-induced seizures and neuronal death in glioma-associated epilepsy patients and in other epilepsy syndromes presenting high System Xc⁻ levels, such as focal cortical dysplasia and tuberous sclerosis (Arena et al., (2019), Brain Pathol 29, 351-365).

International patent application WO 2015/196086 relates to compounds that are stated to be inhibitors of System Xc⁻.

Sulfasalazine is approved for the treatment of disorders, including rheumatoid arthritis, ulcerative colitis, and Crohn's disease. It has been demonstrated to be a non-selective inhibitor of the System Xc⁻ antiporter; however, due to its poor brain exposure, its uses are limited to peripheral indications. In addition, due to its low potency on human System Xc⁻ function, its efficacy in peripheral indications is limited.

There is therefore a need to design new agents that inhibit the System Xc⁻ antiporter, which agents have improved properties and can be used for the treatment of certain cancers, or epilepsy syndromes where system Xc⁻ plays a role.

In order to accelerate the identification of suitable inhibitors of the System Xc⁻ antiporter, there is also a need to develop pharmacological tools that can be used in the development of new biological tests.

### SUMMARY OF THE INVENTION

In a first aspect, the present invention provides a compound of formula (I) or a pharmaceutically acceptable salt thereof,

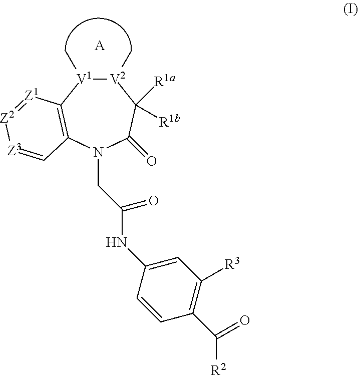

Wherein
A together with the points of attachment V¹ and V² to the remainder of the molecule, represents an optionally substituted aryl or heteroaryl selected from the groups represented by A¹, A² and A³

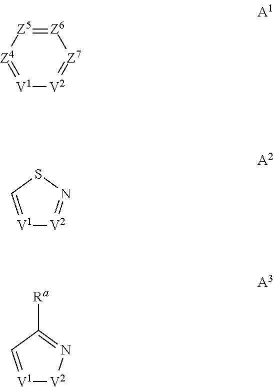

Wherein
V¹ represents C;
V² represents C or N;
Z⁴ represents N or C—R⁷;
Z⁵ represents N or C—R⁸;
Z⁶ represents N or C—R⁹;
Z⁷ represents N or C—R¹⁰;
R^{a} represents halogen;
R⁷, R⁸, R⁹, and R¹⁰ represent independently hydrogen or halogen; or C₁₋₄ alkyl or C₁₋₄ alkoxy, either of which groups may be optionally substituted by one or more substituents; and
Z¹ represents N or C—R⁴;
Z² represents N or C—R⁵;
Z³ represents N or C—R⁶;
R^{1a} and R^{1b} represent independently hydrogen or C₁₋₄ alkyl, either of which groups may be optionally substituted with one or more substituents;
R² represents C₁₋₄ alkyl or C₃₋₇ cycloalkyl, either of which groups may be optionally substituted by one or more substituents;
R³ represents hydrogen, halogen or hydroxyl; or C₁₋₄ alkyl, which group may be optionally substituted by one or more substituents;
R² and R³ together with the phenyl group to which they are attached form an heteroaryl which group is optionally substituted with one or more substituents;
R⁴ and R⁵ represent independently hydrogen, halogen or cyano; or C₁₋₄ alkyl or C₁₋₄ alkoxy, either of which groups may be optionally substituted by one or more substituents; and
R⁶ represents hydrogen, halogen or cyano; or C₁₋₄ alkyl, C₁₋₄ alkoxy, C₃₋₇ heterocycloalkyl, or C₃₋₇ cycloalkyl, any of which groups may be optionally substituted by one or more substitutents.

In a second aspect, the present invention provides for a compound of formula (I′)

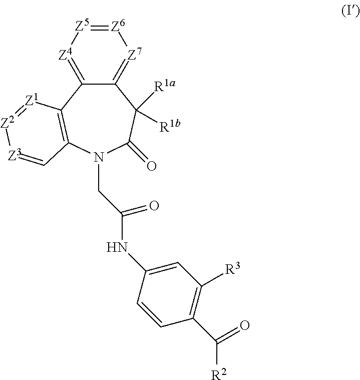

Wherein Z¹, Z², Z³, Z⁴, Z⁵, Z⁶, Z⁷, R¹³, R^{1b}, R² and R³ are as defined here above.
In a third aspect, the present invention provides a compound of formula (I′) or a pharmaceutically acceptable salt thereof,

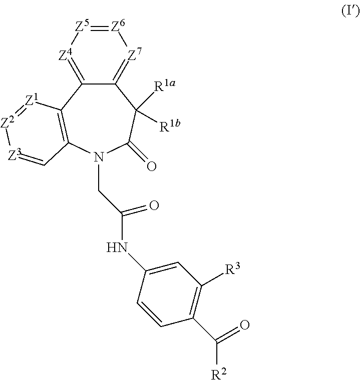

Wherein
Z¹ represents N or C—R⁴;
Z² represents N or C—R⁵;
Z³ represents N or C—R⁶;
Z⁴ represents N or C—R⁷;
Z⁵ represents N or C—R⁸;
Z⁶ represents N or C—R⁹;
Z⁷ represents N or C—R¹⁰;
R^{1a} a and R^{1b} represent independently hydrogen or C₁₋₄ alkyl, either of which groups may be optionally substituted with one or more substituents;
R² represents C₁₋₄ alkyl or C₃₋₇ cycloalkyl, either of which groups may be optionally substituted by one or more substituents;
R³ represents hydrogen, halogen or hydroxyl; or C₁₋₄ alkyl, which group may be optionally substituted by one or more substituents;
R² and R³ together with the phenyl group to which they are attached form an heteroaryl which group is optionally substituted with one or more substituents;
R⁴ and R⁵ represent independently hydrogen, halogen or cyano; or C₁₋₄ alkyl or C₁₋₄ alkoxy, either of which groups may be optionally substituted by one or more substituents;
R⁶ represents hydrogen, halogen or cyano; or C₁₋₄ alkyl, C₁₋₄ alkoxy or C₃₋₇ heterocycloalkyl, any of which groups may be optionally substituted by one or more substitutents; and
R⁷, R⁸, R⁹, and R¹⁰ represent independently hydrogen or halogen; or C₁₋₄ alkyl or C₁₋₄ alkoxy, either of which groups may be optionally substituted by one or more substituents.

In a fourth aspect, the present invention provides a compound of Formula (I), or a pharmaceutically acceptable salt thereof, for use in therapy.

In a fifth aspect, the present invention provides compound of Formula (I), or a pharmaceutically acceptable sale thereof, useful for the treatment of disorders for which system Xc⁻ cystine/glutamate antiporter plays a role.

In particular, the present invention provides compounds of formula (I) which may be useful for the treatment of cancers, or epilepsy syndromes where system Xc⁻ plays a role.

Furthermore, the present invention provides compounds of formula (I) which may be useful to overcome cancer treatment resistance.

In a sixth aspect, the present invention provides a pharmaceutical composition comprising, as an active ingredient, a compound of Formula (I), or a pharmaceutically acceptable salt thereof, in combination with one or more pharmaceutically acceptable carriers, diluents, or excipients.

In a sixth aspect, the present invention provides synthetic intermediates of Formula (II) useful for the chemical synthesis of compounds of formula (I).

### DETAILED DESCRIPTION OF THE INVENTION

The term “C₁₋₄ alkyl” as used herein refers to straight or branched, monovalent, saturated aliphatic hydrocarbon chains of 1 to 4 carbon atoms. Illustrative C₁₋₄ alkyl according to the present invention are methyl and ethyl.

The term “C₁₋₄ alkoxy” represents a group of formula —O—R where R is a “C₁₋₄ alkyl,” as described herein, wherein the C₁-C₄ alkoxy group is connected to the parent structure via the oxygen atom. Suitable alkoxy groups according to the present invention include methoxy.

The term “C₃₋₇ cycloalkyl” as used herein refers to monovalent groups of 3 to 7 carbon atoms derived from a saturated monocyclic hydrocarbon. Illustrative C₃₋₇ cycloalkyl groups include cyclopropyl.

The term “C₃₋₇ heterocycloalkyl” as used herein refers to saturated monocyclic and bicyclic rings containing 3 to 7 carbon atoms and at least one heteroatom selected from oxygen, sulphur and nitrogen. Suitable C₃₋₇ heterocycloalkyl according to the present invention include azetidinyl and azaspiro[2.3]hexan-5-yl.

The terms “Halo,” “halogen,” and “halide” are used indifferently and represent a chloro, fluoro, bromo, or iodo atom. Suitable examples of halogens according to the present invention include chloro and fluoro.

The term “heteroaryl” as used herein represents aromatic carbocyclic groups of from 5 to 14 carbon atoms, having at least an aromatic single ring or multiple condensed rings, wherein one or more of the said carbon atoms have been replaced by one or more heteroatoms selected from oxygen, sulphur and nitrogen. Examples of heteroaryl according to the present invention are optionally substituted benzofuran-3-one or indan-1-one.

For the avoidance of doubt, when reference is made to the compounds of formula (I) this also embraces compounds of formulae (I′) and (IA).

Where any of the groups in the compounds of formula (I) above is stated to be optionally substituted, this group may be unsubstituted, or substituted by one or more substituents. Typically, such groups will be unsubstituted, or substituted by one, two or three substituents. In one embodiment, such groups are unsubstituted. Suitable substituents for each of the groups present on compounds of formula (I) are further described here after in the present specification.

Formula (I) and the formulae depicted hereinafter are intended to represent all individual stereoisomers and all possible mixtures thereof, unless stated or shown otherwise.

Stereoisomers of compounds of formula (I) include cis and trans isomers, optical isomers, diastereomers, geometric isomers, rotational isomers, atropisomers, and conformational isomers of the compounds of formula (I), including compounds exhibiting more than one type of isomerism; and mixtures thereof (such as racemates and diastereomeric pairs).

Compounds of Formula (I) and/or their intermediates may have at least one stereogenic center in their structure. This stereogenic center may be present in a R or a S configuration (referred to as aR or aS for atropisomers), said R and S (or aR and aS) notation is used in correspondence with the rules described in Pure Appl. Chem., 45 (1976) 11-30. The invention thus also relates to all stereoisomeric forms such as enantiomeric and diastereoisomeric forms of the compounds of Formula (I′) and Formula (I) or mixtures thereof (including all possible mixtures of stereoisomers).

With respect to the present invention reference to a compound or compounds is intended to encompass that compound in each of its possible isomeric forms and mixtures thereof, unless the particular isomeric form is specifically referred to.

The carbon-carbon bonds of the compounds of formula (I) are depicted herein using a solid line ( [symbol] ) a solid wedge ( [symbol] ) or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of formula (I′) and formula (I) may contain more than one asymmetric carbon atom. In those compounds, the use of a solid line to depict bonds to asymmetric carbon atoms is meant to indicate that all possible stereoisomers are meant to be included.

Some compounds of formula (I) may exist as single atropisomer or as mixture of atropisomers.

Atropisomers are stereoisomers arising because of hindered rotation about a single bond, where energy differences due to steric strain or other contributors create a barrier to rotation that is high enough to allow for isolation of individual conformers (see for example Bringmann G. et al. Atroposelective Synthesis of Axially Chiral Biaryl Compounds. Angewandte Chemie International Edition. (2005) 44 (34): 5384-5427).

Unlike compounds with classical chiral centers, which racemize via a bond breaking and making process, atropisomers racemize via an intramolecular dynamic process that only involves bond rotation. Depending on rotation barrier, one particular conformer of compounds formula (I) can be in equilibrium with another conformer and thus the composition of the conformers may change with time or condition to reach an equilibrium. The conformation of the compounds of formula (I) can be represented with solid line ( [symbol] ) and/or with solid wedge ( [symbol] ). An example is displayed herebelow with a particular sub-group of compounds of Formula (I), which atropisomers are represented respectively by formula (I-a) and (I-b).

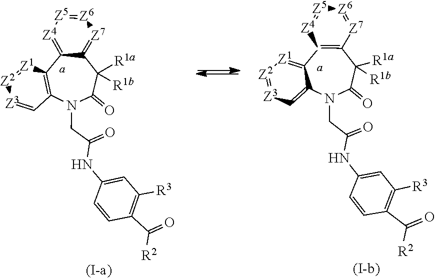

The use of the solid wedge [symbol] on the A and B rings is meant to indicate a conformation associated to the specific atropisomer (I-a) or (I-b).

Some of the compounds of formula (I) may exist in tautomeric forms. Such forms although not explicitly indicated in the above formula are intended to be included within the scope of the present invention. Examples of tautomers include keto (CH₂C═O)↔enol (CH═CHOH) tautomers or amide (NHC═O)↔hydroxyamine (N═COH) tautomers or 2-hydroxypyridine↔pyridinone. Formula (I) and the formulae depicted hereinafter are intended to represent all individual tautomers and all possible mixtures thereof, unless stated or shown otherwise.

It is also to be understood that each individual atom present in formula (I), or in the formula depicted hereinafter, may in fact be present in the form of any of its naturally occurring isotopes, with the most abundant isotope(s) being preferred.

Thus, by way of example, each individual hydrogen atom present in formula (I), or in the formula depicted hereinafter, may be present as a ¹H, ²H (deuterium) or ³H (tritium) atom, preferably ¹H or ²H. Similarly, by way of example, each individual carbon atom present in formula (I′), formula (I), or in the formulae depicted hereinafter, may be present as a ¹¹C, ¹²C, ¹³C or ¹⁴C atom, preferably ¹²C. Similarly, by way of example, each individual fluorine atom may be present as ¹⁸F or ¹⁹F.

Thus, the present invention, also includes within its scope, isotopically-labelled compounds of Formula (I).

Specific embodiments of compounds of formula (I) according to the present invention are described hereafter.

In one embodiment, A represents A¹. In one aspect of this embodiment, V¹ and V² represent C. In another embodiment A represents A². In one aspect of this embodiment, V¹ and V² represent C. In a further embodiment, A represents A³. In one aspect of this embodiment, V¹ represents C and V² represents N.

Generally, R^{a} represents halogen. Particularly R^{a} represents fluoro.

In a first embodiment, R^{1a} represents hydrogen. In a second embodiment, R^{1a} represents optionally substituted C₁₋₄ alkyl. In one aspect of this embodiment, R^{1b} represents optionally substituted methyl.

In a first embodiment, R^{1b} represents hydrogen. In a second embodiment, R^{1b} represents optionally substituted C₁₋₄ alkyl. In one aspect of this embodiment, R^{1b} represents optionally substituted methyl.

Suitably R^{1a} and R^{1b} represent independently hydrogen or C₁₋₄ alkyl.

Illustratively, R^{1a} and R^{1b} represent independently hydrogen or methyl.

In a particular embodiment according to the present invention, R^{1a} represents C₁₋₄ alkyl and R^{1b} represents hydrogen.

In a first embodiment, R² represents optionally substituted C₁₋₄ alkyl. In a first aspect of this embodiment R² represents optionally substituted methyl. In a second aspect of this embodiment, R² represents optionally substituted ethyl. In a second embodiment, R² represents optionally substituted C₃₋₇ cycloalkyl. In a first aspect of this embodiment, R² represents optionally substituted cyclopropyl.

Suitably, R² represents C₁₋₄ alkyl or C₃₋₇ cycloalkyl.

Illustratively, R² represents methyl or cyclopropyl.

In a first embodiment, R³ represents hydrogen. In a second embodiment, R³ represents halogen.

In one aspect of this embodiment, R³ represents fluoro. In a third embodiment, R³ represents hydroxyl. In a fourth embodiment, R³ represents optionally substituted C₁₋₄ alkyl. In one aspect of this embodiment, R³ represents optionally substituted methyl.

Suitably, R³ represents hydrogen.

In a particular embodiment according to the invention, R² and R³ together with the phenyl group to which they are attached form an optionally substituted benzofuran-3-one or indan-1-one.

Illustratively, R² and R³ together with the phenyl group to which they are attached form benzofuran-3-one.

In one embodiment, Z¹ represents N. In another embodiment, Z¹ represents C—R⁴.

In one embodiment, Z² represents N. In another embodiment, Z² represents C—R⁵.

In one embodiment, Z³ represents N. In another embodiment, Z³ represents C—R⁶.

In one embodiment, Z⁴ represents N. In another embodiment, Z⁴ represents C—R⁷.

In one embodiment, Z⁵ represents N. In another embodiment, Z⁵ represents C—R⁸.

In one embodiment, Z⁶ represents N. In another embodiment, Z⁶ represents C—R⁹.

In one embodiment, Z⁷ represents N. In another embodiment, Z⁷ represents C—R¹⁰.

In a particular embodiment according to the invention, one or none of Z¹, Z², and Z³ represents N. In one aspect of this particular embodiment, Z² represents N, Z¹ represents C—R⁴ and Z³ represents C—R⁶.

In a particular embodiment according to the invention, one or none of Z⁴, Z⁵, Z⁶ and Z⁷ represents N. In one aspect of this particular embodiment, Z⁷ represents N, Z⁴ represents C—R⁷, Z⁵ represents C—R⁸, Z⁶ represents C—R⁹.

In a first embodiment, R⁴ represents hydrogen. In a second embodiment, R⁴ represents halogen.

In a first aspect of this embodiment, R⁴ represents fluoro. In a second aspect of this embodiment, R⁴ represents chloro. In a third embodiment, R⁴ represents cyano. In a fourth embodiment, R⁴ represents optionally substituted C₁₋₄ alkyl. In one aspect of this embodiment, R⁴ represents optionally substituted methyl.

In a fifth embodiment, R⁴ represents optionally substituted C₁₋₄ alkoxy. In a first aspect of this embodiment, R⁴ represents optionally substituted methoxy.

Suitably, R⁴ represents hydrogen, halogen, cyano, C₁₋₄ alkyl or C₁₋₄ alkoxy.

Illustratively, R⁴ represents hydrogen, fluoro, chloro, or methoxy.

In a first embodiment, R⁵ represents hydrogen. In a second embodiment, R⁵ represents halogen. In a first aspect of this embodiment, R⁵ represents fluoro. In a second aspect of this embodiment, R⁵ represents chloro. In a third embodiment, R⁵ represents cyano. In a fourth embodiment, R⁵ represents optionally substituted C₁₋₄ alkyl. In one aspect of this embodiment, R⁵ represents optionally substituted methyl. In a fifth embodiment, R⁵ represents optionally substituted C₁₋₄ alkoxy. In a first aspect of this embodiment, R⁵ represents optionally substituted methoxy.

Suitably, R⁵ represents hydrogen, halogen, cyano, C₁₋₄ alkyl or C₁₋₄ alkoxy.

Illustratively, R⁵ represents hydrogen, fluoro, chloro, or methoxy.

In a first embodiment, R⁶ represents hydrogen. In a second embodiment, R⁶ represents halogen. In a first aspect of this embodiment, R⁶ represents fluoro. In a second aspect of this embodiment, R⁶ represents chloro. In a third embodiment, R⁶ represents cyano. In a fourth embodiment, R⁶ represents optionally substituted C₁₋₄ alkyl. In a first aspect of this embodiment, R⁶ represents optionally substituted methyl. In a fifth embodiment, R⁶ represents optionally substituted C₁₋₄ alkoxy. In a first aspect of this embodiment, R⁶ represents optionally substituted methoxy. In a sixth embodiment, R⁶ represents optionally substituted C₃₋₇ heterocycloalkyl. In a first aspect of this embodiment, R⁶ represents optionally substituted azetidinyl. In a second aspect of this embodiment, R⁶ represents optionally substituted azaspiro[2.3]hexanyl. In a seventh embodiment, R⁶ represents optionally substituted C₃₋₇ cycloalkyl. In a first aspect of this embodiment, R⁶ represents optionally substituted cyclobutane.

Suitably, R⁶ represents hydrogen, halogen, cyano, C₁₋₄ alkyl, or C₁₋₄ alkoxy; or optionally substituted C₃₋₇ heterocycloalkyl or C₃₋₇ cycloalkyl.

More suitably, R⁶ represents hydrogen, halogen, cyano, C₁₋₄ alkyl, or C₁₋₄ alkoxy; or optionally substituted C₃₋₇ heterocycloalkyl.

Ideally, R⁶ represents C₁₋₄ alkyl, C₁₋₄ alkoxy or optionally substituted C₃₋₇ heterocycloalkyl.

Illustratively, R⁶ represents methyl, methoxy, optionally substituted azetidinyl, optionally substituted azaspiro[2.3]hexanyl or optionally substituted cyclobutane.

Particularly, R⁶ represents methyl, methoxy, 3,3-difluoroazetidin-1-yl, 2,2-difluoro-5-azaspiro[2.3]hexan-5-yl, 3-fluoro-azetidin-1-yl, 3,3-(hydroxy)(methyl)-azetidin-1-yl, 3,3-(fluoro)(methyl)azetidin-1-yl, cyclobutane, or 1-hydroxy-cyclobutane.

More particularly, R⁶ represents methyl, methoxy, 3,3-difluoroazetidin-1-yl or 2,2-difluoro-5-azaspiro[2.3]hexan-5-yl,

In a first embodiment, R⁷ represents hydrogen. In a second embodiment, R⁷ represents halogen. In a first aspect of this embodiment, R⁷ represents fluoro. In a third embodiment, R⁷ represents optionally substituted C₁₋₄ alkyl. In a first aspect of this embodiment, R⁷ represents optionally substituted methyl. In a fourth embodiment, R⁷ represents optionally substituted C₁₋₄ alkoxy. In a first aspect of this embodiment, R⁷ represents optionally substituted methoxy.

Suitably, R⁷ represents hydrogen, halogen, C₁₋₄ alkyl or C₁₋₄ alkoxy.

Illustratively, R⁷ represents hydrogen.

In a first embodiment, R⁸ represents hydrogen. In a second embodiment, R⁸ represents halogen. In a first aspect of this embodiment, R⁸ represents fluoro. In a third embodiment, R⁸ represents optionally substituted C₁₋₄ alkyl. In a first aspect of this embodiment, R⁸ represents optionally substituted methyl. In a fourth embodiment, R⁸ represents optionally substituted C₁₋₄ alkoxy. In a first aspect of this embodiment, R⁸ represents optionally substituted methoxy.

Suitably, R⁸ represents hydrogen, halogen, C₁₋₄ alkyl or C₁₋₄ alkoxy.

Illustratively, R⁸ represents hydrogen, fluoro or methoxy.

In a first embodiment, R⁹ represents hydrogen. In a second embodiment, R⁹ represents halogen. In a first aspect of this embodiment, R⁹ represents fluoro. In a third embodiment, R⁹ represents optionally substituted C₁₋₄ alkyl. In a first aspect of this embodiment, R⁹ represents optionally substituted methyl. In a fourth embodiment, R⁹ represents optionally substituted C₁₋₄ alkoxy. In a first aspect of this embodiment, R⁹ represents optionally substituted methoxy.

Suitably, R⁹ represents hydrogen, halogen, C₁₋₄ alkyl or C₁₋₄ alkoxy.

Illustratively, R⁹ represents hydrogen.

In a first embodiment, R¹⁰ represents hydrogen. In a second embodiment, R¹⁰ represents halogen. In a first aspect of this embodiment, R¹⁰ represents fluoro. In a third embodiment, R¹⁰ represents optionally substituted C₁₋₄ alkyl. In a first aspect of this embodiment, R¹⁰ represents optionally substituted methyl. In a fourth embodiment, R¹⁰ represents optionally substituted C₁₋₄ alkoxy. In a first aspect of this embodiment, R¹⁰ represents optionally substituted methoxy.

Suitably, R¹⁰ represents hydrogen, halogen, C₁₋₄ alkyl or C₁₋₄ alkoxy.

Illustratively, R¹⁰ represents hydrogen.

Suitable optional substitutents on R¹³, Rib, R², R³, R⁴, R⁵, R⁶, R⁷, R⁸, R⁹, and R¹⁰ are hydroxyl, halogen, C₁₋₄ alkyl, or C₁₋₄ alkoxy. Particular examples of optional substituents on R², R³, R⁴, R⁵, R⁶, R⁷, R⁸, R⁹, and R¹⁰ are hydroxyl, fluoro, methyl and methoxy.

Illustratively, R^{1a}, R^{1b}, R², R³, R⁴, R⁵, R⁷, R⁸, R⁹, and R¹⁰ are not substituted.

In a first particular embodiment, the present invention relates to a particular subclass of compounds of formula (I) represented by formula (IA),

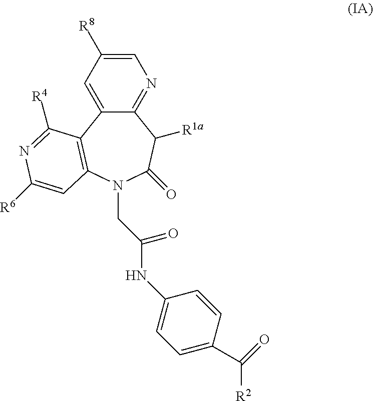

Wherein R^{1a}, R², R⁴, R⁶ and R⁸ are as defined above.

In compounds according to formula (IA), suitably:
R^{1a} represents hydrogen or C₁₋₄ alkyl;
R² represents C₁₋₄ alkyl or C₃₋₇ cycloalkyl;
R⁴ and R⁸ represent independently hydrogen, halogen, or C₁₋₄ alkoxy; and
R⁶ represents C₁₋₄ alkyl, C₁₋₄ alkoxy or C₃₋₇ heterocycloalkyl.

In compounds according to formula (IA), suitably:
R^{1a} represents hydrogen or methyl;
R² represents methyl or cyclopropyl;
R⁴ represents hydrogen, chloro, fluoro or methoxy;
R⁶ represents methyl, methoxy, 2,2-difluoro-azetidin-1-yl, or 2,2-difluoro-5-azaspiro[2.3]hexan-5-y, 3-fluoroazetidin-1-yl, 3,3-(hydroxy)(methyl)-azetidin-1-yl, 3,3-(fluoro)(methyl)azetidin-1-yl, cyclobutane, or 1-hydroxy-cyclobutane; and
R⁸ represents hydrogen, fluoro, or methoxy.

In compounds according to formula (IA), typically:
R^{1a} represents hydrogen or C₁₋₄ alkyl;
R² represents C₁₋₄ alkyl or C₃₋₇ cycloalkyl;
R⁴ and R⁸ represent independently hydrogen, halogen, or C₁₋₄ alkoxy; and
R⁶ represents C₁₋₄ alkyl, C₁₋₄ alkoxy or C₃₋₇ heterocycloalkyl.

In compounds according to formula (IA), suitably:
R^{1a} represents hydrogen or methyl;
R² represents methyl or cyclopropyl;
R⁴ represents hydrogen, chloro, fluoro or methoxy;
R⁶ represents methyl, methoxy, 2,2-difluoro-azetidin-1-yl, or 2,2-difluoro-5-azaspiro[2.3]hexan-5-yl; and
R⁸ represents hydrogen, fluoro, or methoxy.

In one particular aspect of compounds of formula (IA), R⁸ represents fluoro.

Specific novel compounds in accordance with the present invention include each of the compounds whose preparation is described in the accompanying Examples, their individual stereoisomers, and pharmaceutically acceptable salts and solvates thereof.

In a particular embodiment, the present invention relates to compounds of formula (I) selected from the group consisting of:
N-(4-acetylphenyl)-2-[3-fluoro-5,10-dimethyl-9-oxo-4,8,12-triazatricyclo[9.4.0.0^{2,7}]pentadeca-1(11),2,4,6,12,14-hexaen-8-yl]acetamide;
N-[4-(cyclopropanecarbonyl)phenyl]-2-[3-fluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[3,14-difluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[14-fluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[(10R)-3-chloro-14-fluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[3-chloro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[5-(3,3-difluoroazetidin-1-yl)-3-fluoro-10-methyl-9-oxo-4,8,12-triazatricyclo[9.4.0.0²⁷1]pentadeca-1(11),2,4,6,12,14-hexaen-8-yl]acetamide;
N-(4-acetylphenyl)-2-[3-fluoro-5-(3-fluoroazetidin-1-yl)-10-methyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[3-chloro-5-(2,2-difluoro-5-azaspiro[2.3]hexan-5-yl)-9-oxo-4,8,12-triazatricyclo[9.4.0.0^{2,7}]pentadeca-1(11),2,4,6,12,14-hexaen-8-yl]acetamide; and
N-(4-acetylphenyl)-2-(1-fluoro-3-methyl-6-oxo-7H-pyrido[4,3-d][3]benzazepin-5-yl)acetamide,
N-(4-acetylphenyl)-2-[3-fluoro-5-methoxy-10-methyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[14-methoxy-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[3-methoxy-5,10-dimethyl-9-oxo-4,8,12-triazatricyclo[9.4.0.0^{2,7}]pentadeca-1(15),2(7),3,5,11,13-hexaen-8-yl]acetamide;
N-(4-acetylphenyl)-2-(3-methoxy-10-methyl-9-oxo-4,8,12-triazatricyclo[9.4.0.0^{2,7}]pentadeca-1(11),2(7),3,5,12,14-hexaen-8-yl)acetamide;
2-[3-fluoro-5,10-dimethyl-9-oxo-4,8,12-triazatricyclo[9.4.0.0^{2,7}]pentadeca-1(11),2,4,6,12,14-hexaen-8-yl]-N-(4-propanoylphenyl)acetamide;
N-(4-acetyl-3-hydroxy-phenyl)-2-[3-fluoro-5,10-dimethyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-(3,14-difluoro-5-methoxy-10-methyl-9-oxo-4,8,12-triazatricyclo[9.4.0.0^{2,7}]pentadeca-1(11),2(7),3,5,12,14-hexaen-8-yl)acetamide;
2-[3,14-difluoro-5,10-dimethyl-9-oxo-4,8,12-triazatricyclo[9.4.0.0^{2,7}]pentadeca-1(11),2(7),3,5,12,14-hexaen-8-yl]-N-(3-oxobenzofuran-6-yl)acetamide;
N-(4-acetylphenyl)-2-(10-cyano-2-fluoro-5,9-dimethyl-6-oxo-5H-pyrido[2,3-d][1]benzazepin-7-yl)acetamide;
N-(4-acetylphenyl)-2-[(10)-3-fluoro-14-methoxy-5,10-dimethyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[3-fluoro-5-(3-fluoroazetidin-1-yl)-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[5-(3,3-difluoroazetidin-1-yl)-3-fluoro-9-oxo-4,8,12-triazatricyclo[9.4.0.0^{2,7}]pentadeca-1(11),2(7),3,5,12,14-hexaen-8-yl]acetamide;
N-(4-acetylphenyl)-2-(3,5-difluoro-10-methyl-9-oxo-4,8,12-triazatricyclo[9.4.0.0^{2,7}]pentadeca-1(11),2(7),3,5,12,14-hexaen-8-yl)acetamide;
N-(4-acetyl-3-fluoro-phenyl)-2-[(10)-3-fluoro-5,10-dimethyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[3-fluoro-5-(3-hydroxy-3-methyl-azetidin-1-yl)-10-methyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[3-fluoro-5-(3-fluoro-3-methyl-azetidin-1-yl)-10-methyl-9-oxo-4,8,12-triazatricyclo[9.4.0.0^{2,7}]pentadeca-1(11).2(7).3,5,12,14-hexaen-8-yl]acetamide;
N-(4-acetylphenyl)-2-[3-fluoro-5-(3-hydroxy-3-methyl-azetidin-1-yl)-9-oxo-4,8,12-triazatricyclo[9.4.0.0^{2,7}]pentadeca-1(11),2(7),3,5,12,14-hexaen-8-yl]acetamide;
N-(4-acetylphenyl)-2-(14-fluoro-7,12-dimethyl-8-oxo-4-thia-5,9,13-triazatricyclo[8.4.0.0^{2,6}]tetradeca-1(10),2,5,11,13-pentaen-9-yl)acetamide;
N-(4-acetylphenyl)-2-[3-fluoro-5-(1-hydroxycyclobutyl)-10-methyl-9-oxo-4,8,12-triazatricyclo[9.4.0.0^{2,7}]pentadeca-1(15),2,4,6,11,13-hexaen-8-yl]acetamide;
N-(4-acetylphenyl)-2-[3,14-difluoro-5-(hydroxymethyl)-10-methyl-9-oxo-4,8,12-triazatricyclo[9.4.0.0^{2,7}]pentadeca-1(11),2,4,6,12,14-hexaen-8-yl]acetamide;
N-(4-acetylphenyl)-2-(5-cyclobutyl-3-fluoro-10-methyl-9-oxo-4,8,12-triazatricyclo[9.4.0.0^{2,7}]pentadeca-1(11),2(7),3,5,12,14-hexaen-8-yl)acetamide;
N-(4-acetylphenyl)-2-(14-fluoro-5-methoxy-10-methyl-9-oxo-4,8,12-triazatricyclo[9.4.0.0^{2,7}]pentadeca-1(11),2(7),3,5,12,14-hexaen-8-yl)acetamide;
N-(4-acetyl-3-hydroxy-phenyl)-2-(3,14-difluoro-5,10-dimethyl-9-oxo-4,8,12-triazatricyclo[9.4.0.0^{2,7}]pentadeca-1(11),2(7),3,5,12,14-hexaen-8-yl)acetamide;
N-(4-acetylphenyl)-2-(3,14-difluoro-5,10-dimethyl-9-oxo-4,8,13-triazatricyclo[9.4.0.0^{2,7}]pentadeca-1(11),2(7),3,5,12,14-hexaen-8-yl)acetamide;
N-(4-acetylphenyl)-2-[11-fluoro-9-(3-fluoroazetidin-1-yl)-5-methyl-6-oxo-5H-pyrido[2,3-d][1]benzazepin-7-yl]acetamide;
N-(4-acetylphenyl)-2-(14-chloro-4-fluoro-7,12-dimethyl-8-oxo-5,6,9,13-tetrazatricyclo[8.4.0.0²′6]tetradeca-1(14),2,4,10,12-pentaen-9-yl)acetamide; and enantiomers thereof.

In a particular aspect, the present invention relates to compounds of formula (I) as described in the accompanying Examples 1-36.

In a further particular aspect, the present invention relates to compounds of formula (I) selected from the group consisting of:
N-(4-acetylphenyl)-2-[(10R)-3-fluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[(10R)-3,14-difluoro-5,10-dimethyl-9-oxo-4,8,12-triazatricyclo[9.4.0.0^{2,7}]pentadeca-1(11),2,4,6,12,14-hexaen-8-yl]acetamide;
N-[4-(cyclopropanecarbonyl)phenyl]-2-[(10R)-3-fluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[(10R)-3-chloro-14-fluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[3-chloro-5-(2,2-difluoro-5-azaspiro[2.3]hexan-5-yl)-9-oxo-4,8,12-triazatricyclo[9.4.0.0^{2,7}]pentadeca-1(11),2,4,6,12,14-hexaen-8-yl]acetamide;
N-(4-acetylphenyl)-2-(1-fluoro-3-methyl-6-oxo-7H-pyrido[4,3-d][3]benzazepin-5-yl)acetamide; 2-[(10R)-3-fluoro-5,10-dimethyl-9-oxo-4,8,12-triazatricyclo[9.4.0.0^{2,7}]pentadeca-1(11),2,4,6,12,14-hexaen-8-yl]-N-(4-propanoylphenyl)acetamide;
N-(4-acetylphenyl)-2-[3-fluoro-5-(3-fluoroazetidin-1-yl)-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[5-(3,3-difluoroazetidin-1-yl)-3-fluoro-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[3-fluoro-5-(3-hydroxy-3-methyl-azetidin-1-yl)-10-methyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[3-fluoro-5-(3-fluoro-3-methyl-azetidin-1-yl)-10-methyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[3-fluoro-5-(3-hydroxy-3-methyl-azetidin-1-yl)-9-oxo-4,8,12-triazatricyclo[9.4.0.0^{2,7}]pentadeca-1(11),2(7),3,5,12,14-hexaen-8-yl]acetamide;
N-(4-acetylphenyl)-2-(14-fluoro-7,12-dimethyl-8-oxo-4-thia-5,9,13-triazatricyclo[8.4.0.0^{2,6}]tetradeca-1(10),2,5,11,13-pentaen-9-yl)acetamide;
N-(4-acetylphenyl)-2-[3-fluoro-5-(1-hydroxycyclobutyl)-10-methyl-9-oxo-4,8,12-triazatricyclo[9.4.0.0^{2,7}]pentadeca-1(15),2,4,6,11,13-hexaen-8-yl]acetamide;
N-(4-acetylphenyl)-2-[3,14-difluoro-5-(hydroxymethyl)-10-methyl-9-oxo-4,8,12-triazatricyclo[9.4.0.0^{2,7}]pentadeca-1(11),2,4,6,12,14-hexaen-8-yl]acetamide;
N-(4-acetylphenyl)-2-(3,14-difluoro-5,10-dimethyl-9-oxo-4,8,13-triazatricyclo[9.4.0.0^{2,7}]pentadeca-1(11),2(7),3,5,12,14-hexaen-8-yl)acetamide; and
N-(4-acetylphenyl)-2-(14-chloro-4-fluoro-7,12-dimethyl-8-oxo-5,6,9,13-tetrazatricyclo[8.4.0.0²′6]tetradeca-1(14),2,4,10,12-pentaen-9-yl)acetamide.

The present invention also provides a compound of formula (I) as defined above or a pharmaceutically acceptable salt thereof, for use in therapy.

In particular, the present invention provides a compound of formula (I) as defined above, or a pharmaceutically acceptable salt thereof, for use in the treatment of diseases and/or disorders in which System Xc⁻ plays a role.

In the following aspects, the compound of formula (I) as defined above may be an inhibitor of the System Xc⁻ antiporter.

In a first aspect, the present invention provides a compound of formula (I) as defined above, or a pharmaceutically acceptable salt thereof, for use in the treatment of cancers where System Xc⁻ plays a role, in epilepsy syndromes where System Xc⁻ plays a role, or in cancer treatment resistance.

In a first embodiment according to this aspect, the present invention provides a compound of formula (I) as defined above, or a pharmaceutically acceptable salt thereof, for use in the treatment of cancers where System Xc⁻ plays a role.

In particular, the present invention provides a compound of formula (I) as defined above, or a pharmaceutically acceptable salt thereof for use in the treatment of glioma, colon carcinoma, colorectal carcinoma, lung cancer, esophageal carcinoma, triple negative breast cancer, hepatocellular carcinoma, adrenocortical carcinoma, kidney carcinoma, mesothelioma, sarcoma, uveal melanoma, gastric cancer, pancreatic carcinoma or leukemia.

In a second embodiment according to this aspect, the present invention provides a compound of formula (I) as defined above for use in the treatment of epilepsy syndromes where System Xc⁻ plays a role.

In particular, the present invention provides a compound of formula (I) as defined above, or a pharmaceutically acceptable salt thereof, for use in the treatment of epileptogenesis, glutamate induced seizures, glioma associated epilepsy, focal cortical dysplasia or tuberous sclerosis.

In a third embodiment, the present invention provides compounds of formula (I) for use in the treatment of cancer treatment resistance.

In particular, the present invention provides a compound of formula (I) as defined above, or a pharmaceutically acceptable salt thereof, for use in the treatment of multidrug resistance in several cancer types.

In a second aspect, the present invention provides for the use of a compound of formula (I) as defined above, or a pharmaceutically acceptable salt thereof, for the manufacture of a medicament useful for the treatment of diseases and/or disorders in which system Xc⁻ cystine/glutamate antiporter plays a role.

In a first embodiment of this aspect, the present invention provides for the use of a compound of formula (I) as defined above, or a pharmaceutically acceptable salt thereof, for the manufacture of a medicament useful for the treatment of cancers where System Xc⁻ plays a role.

In particular, the present invention provides for the use of a compound of formula (I) as defined above, or a pharmaceutically acceptable salt thereof for the manufacture of a medicament useful for the treatment glioma, colon carcinoma, colorectal carcinoma, lung cancer, esophageal carcinoma, triple negative breast cancer, hepatocellular carcinoma, adrenocortical carcinoma, kidney carcinoma, mesothelioma, sarcoma, uveal melanoma, gastric cancer, pancreatic carcinoma or leukemia.

In a second embodiment according to this aspect, the present invention provides for the use of a compound of formula (I) as defined above, or a pharmaceutically acceptable salt thereof, for the manufacture of a medicament useful for the treatment of epilepsy syndromes where System Xc⁻ plays a role.

In particular, the present invention provides a compound of formula (I) as defined above, or a pharmaceutically acceptable salt thereof, for the manufacture of a medicament useful for the treatment of epileptogenesis, glutamate induced seizures, glioma associated epilepsy, focal cortical dysplasia or tuberous sclerosis.

In a third embodiment according to this aspect, the present invention provides for the use of a compound of formula (I) as defined above, or a pharmaceutically acceptable salt thereof, for the manufacture of a medicament useful for cancer treatment resistance.

In particular, the present invention provides for the use of a compound of formula (I) as defined above, or a pharmaceutically acceptable salt thereof, for the manufacture of a medicament useful for the treatment of multidrug resistance in several cancer types. In a third aspect, the present invention provides a method for the treatment of disorders for which the administration of inhibitors of the System Xc⁻ is indicated, which comprises administering to a patient in need of such treatment an effective amount of a compound of formula (I) as defined above, or a pharmaceutically acceptable salt thereof.

In a first embodiment according to this aspect, the present invention provides a method for the treatment of cancers where System Xc⁻ plays a role, which comprises administering to a patient in need of such treatment an effective amount of a compound of formula (I) as defined above, or a pharmaceutically acceptable salt thereof.

In particular, the present invention provides a method for the treatment of glioma, colon carcinoma, colorectal carcinoma, lung cancer, esophageal carcinoma, triple negative breast cancer, hepatocellular carcinoma, adrenocortical carcinoma, kidney carcinoma, mesothelioma, sarcoma, uveal melanoma, gastric cancer, pancreatic carcinoma or leukemia, which comprises administering to a patient in need of such treatment of an effective amount of a compound of formula (I) as defined above, or a pharmaceutically acceptable salt thereof.

In a second embodiment according to this aspect, the present invention provides a method for the treatment of epilepsy syndromes where System Xc⁻ plays a role, which comprises administering to a patient in need of such treatment an effective amount of a compound of formula (I) as defined above, or a pharmaceutically acceptable salt thereof.

In particular, the present invention provides a method for the treatment of epileptogenesis, glutamate induced seizures, glioma associated epilepsy, focal cortical dysplasia or tuberous sclerosis, which comprises administering to a patient in need of such treatment an effective amount of a compound of formula (I) as defined above, or a pharmaceutically acceptable salt thereof.

In a third embodiment according to this aspect, the present invention provides a method for the treatment of cancer treatment resistance, which comprises administering to a patient in need of such treatment an effective amount of a compound of formula (I) as defined above, or a pharmaceutically acceptable salt thereof. In particular, the present invention provides a method for the treatment of multidrug resistance in several cancer types, which comprises administering to a patient in need of such treatment an effective amount of a compound of formula (I) as defined above, or a pharmaceutically acceptable salt thereof.

As used herein, the term “patient” refers to a mammal that is afflicted with one or more disorders associated with function or expression of System Xc⁻. It will be understood that the most preferred patient is a human.

It is also recognized that one skilled in the art may affect the disorders by treating a patient presently afflicted with the disorders, or by prophylactically treating a patient afflicted with the disorders with an effective amount of the compound of Formula (I). Thus, the terms “treatment” and “treating” are intended to refer to all processes wherein there may be a slowing, interrupting, arresting, controlling, or stopping of the progression of the disorders described herein, and is intended to include prophylactic treatment of such disorders, but does not necessarily indicate a total elimination of all disorder symptoms.

Activity in any of the above-mentioned therapeutic indications or disorders can of course be determined by carrying out suitable clinical trials in a manner known to a person skilled in the relevant art for the particular indication and/or in the design of clinical trials in general.

For use in medicine, the salts of the compounds of formula (I) will be pharmaceutically acceptable salts. Other salts may, however, be useful in the preparation of the compounds of use in the invention or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compound of formula (I) or formula (I) include acid addition salts which may, for example, be formed by mixing a solution of the compound of formula (I) with a solution of a pharmaceutically acceptable acid.

The present invention includes within its scope solvates of the compounds of formula (I) above. Such solvates may be formed with common organic solvents or water.

The present invention also includes within its scope co-crystals of the compounds of formula (I) above. The technical term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity (see Pharmaceutical Salts and Co-crystals, ed. J. Wouters & L. Quere, RSC Publishing, 2012).

Compounds according to the present invention may exist in different polymorphic forms. Although not explicitly indicated in the above formula, such forms are intended to be included within the scope of the present invention.

The invention also includes within its scope pro-drug forms of the compounds of formula (I) and its various sub-scopes and sub-groups.

For treating diseases, compounds of formula (I) or their pharmaceutically acceptable salts may be employed at an effective daily dosage and administered in the form of a pharmaceutical composition.

Therefore, another embodiment of the present invention concerns a pharmaceutical composition comprising an effective amount of a compound of formula (I) or a pharmaceutically acceptable salt thereof in combination with a pharmaceutically acceptable diluent or carrier.

To prepare a pharmaceutical composition according to the invention, one or more of the compounds of formula (I) or a pharmaceutically acceptable salt thereof is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, parenteral, intranasal, or intratumoral.

Pharmaceutical compositions comprising compounds according to the invention can, for example, be administered orally, parenterally, i.e. intravenously, intramuscularly or subcutaneously, intrathecally, by inhalation, intranasally or by ophthalmic administration.

Pharmaceutical compositions suitable for oral administration can be solids or liquids and can, for example, be in the form of tablets, pills, dragees, gelatin capsules, solutions, syrups, chewing-gums and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycolumn or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus, the quantity of compound of formula (I) in compositions for oral administration is at least 0.5% by weight and can be up to 80% by weight with respect to the total weight of the composition.

In accordance with the invention, it has also been found that the compounds of formula (I) or the pharmaceutically acceptable salts thereof can be administered alone or in combination with other pharmaceutically active ingredients.

In particular, compounds of formula (I) according to the present invention could be combined with other active ingredients that increase intracellular reactive oxygen species, regulate amino acid metabolism or with immunotherapeutic agents.

In compositions for parenteral administration, the quantity of compound of formula (I) present is at least 0.5% by weight and can be up to 33% by weight with respect to the total weight of the composition. For the preferred parenteral compositions, the dosage unit is in the range 0.5 mg to 3000 mg of compounds of formula (I).

The daily dose can fall within a wide range of dosage units of compound of formula (I) and is generally in the range 0.5 to 3000 mg. However, it should be understood that the specific doses can be adapted to particular cases depending on the individual requirements, at the physician's discretion.

### SYNTHETIC SCHEMES

It will be apparent to the person skilled in the art that there are various synthetic pathways that can lead to the compounds according to the invention. The following processes are aimed at illustrating some of these synthetic pathways but should not be construed in any way as a limitation on how the compounds according to the invention should be made.

During any of the below synthetic sequences, it may be necessary and/or desirable to protect sensitive or reactive groups on any of the molecules concerned. This may be achieved by means of conventional protecting groups (PG), such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} edition, 1999. The protecting groups may be removed at any convenient subsequent stage utilising methods known from the art.

The compounds of Formula (I) according to the invention can be prepared analogously to conventional methods as understood by the person skilled in the art of synthetic organic chemistry.

In the following description of general synthetic methods, “DCM” means dichloromethane; “DIPEA” refers to N,N-di-iso-propylethylamine; “DMF” refers to N,N-dimethylformamide; “DMSO” refers to dimethylsulfoxide; “EDC” refers to 1-Ethyl-3-carbodiimide hydrochloride; “; “TEA” refers to triethylamine; “THF” refers to tetrahydrofuran; “HATU” refers to hexafluorophosphate azabenzotriazole tetramethyl uranium; “HBTU” refers to hexafluorophosphate benzotriazole tetramethyl uronium; “HOBt” refers to hydroxybenzotriazole”; “TCFH” refers to chloro-N,N,N′,N′-tetramethylformamidinium hexafluorophosphate; and “NMI” refers to N-methylimidazole.

The following description of synthetic schemes provides for means of preparing compounds of formula (I). However, analogous methods may be used in the preparation of compounds of formula (I). According to one embodiment, compounds having the general Formula (I), wherein A represents A¹ may be prepared by reaction of a compound of formula (2) with an amide of formula (4) or by reaction of a compounds of formula (3) with an aromatic amine of formula (5) according to the equation:

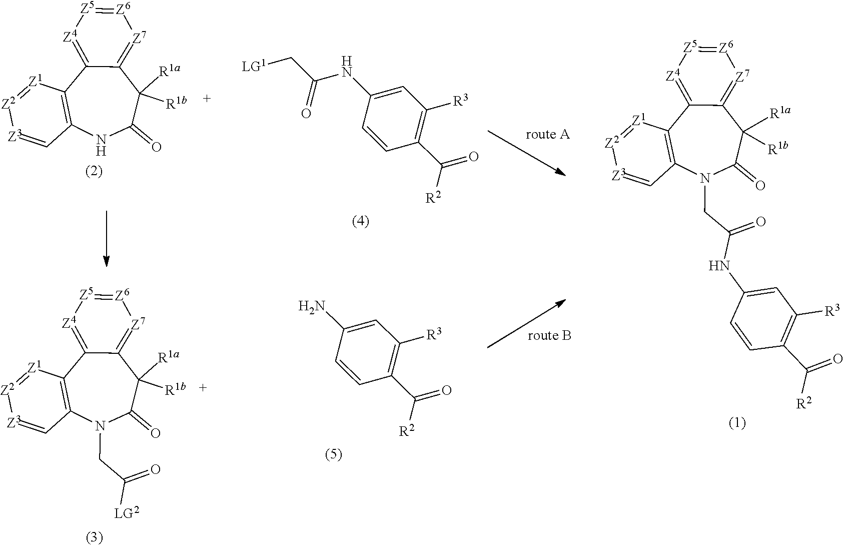

wherein Z¹, Z², Z³, Z⁴, Z⁵, Z⁶, Z⁷, R^{1a}, R^{1b} and R² are as defined above for compound of Formula (I) and where LG¹ is a halogen atom or a leaving group such as a mesylate or tosylate; and LG² is hydroxy, alkoxy, or halogen.
   The reaction following route A may be performed with a base such as trialkyl amines, inorganic carbonates or pyridines, with or without the presence of an iodide salt such as KI or NaI in a suitable solvent such as DMSO, DMF, sulfolane, acetonitrile, or THE.
   Alternatively, compounds of Formula (I) may be prepared following route B by reaction of a carboxylic acid or carboxylic derivatives of formula (3) with aromatic amines (5) following procedures for the formation of an amide from carboxylic acids or carboxylic derivatives and amines known to the person skilled in the art. The reaction following route B, when LG² is a halogen such as chlorine, may be performed with a base such as trialkyl amines, inorganic carbonates, or pyridines in a suitable solvent such as DCM, DMSO, DME, sulfolane, acetonitrile, or THE. When LG² is hydroxy, the reaction may be performed with similar bases and in the presence of an amide coupling reagent such as HBTU, HATU, TCFH/NMI, EDC/HOBt, or according to any other method known to the person skilled in the art. Alternatively, compounds of formula (3) in which LG² is hydroxy can be transformed into compounds of formula (3) in which LG² is chloro by reaction with sulfonyl chloride or thionyl chloride in the presence or absence of catalytic DMF, in a suitable solvent such as DCM or THF at room or at higher temperatures such as 70° C.

Compounds of formula (3) where LG² is an alkoxy such as OMe, OEt, or OtBu may be prepared by reaction of intermediate (2) with an alpha-chloro ester or an alpha-bromo ester such as methyl 2-bromoacetate, ethyl 2-bromoacetate, or tert-butyl 2-bromoacetate in the presence of a base such as potassium carbonate in a polar solvent such as DMF at room temperature or by any method known to the person skilled in the art. Further basic or acid ester hydrolysis known to the person skilled in the art may be used to form compounds of formula (3) in which LG² is OH.

Alternatively, compounds of Formula (I) may be prepared by reaction of an intermediate of Formula (3) wherein LG² is NH₂, hereinafter referred to as (3′), with a compound of formula (5′) wherein X is sulfonate such as a triflate, an halogen such as chloro or bromo in the presence of a catalytic amount of a palladium catalyst. This reaction, the “Buchwald amide coupling”, is known to the person skilled in the art.

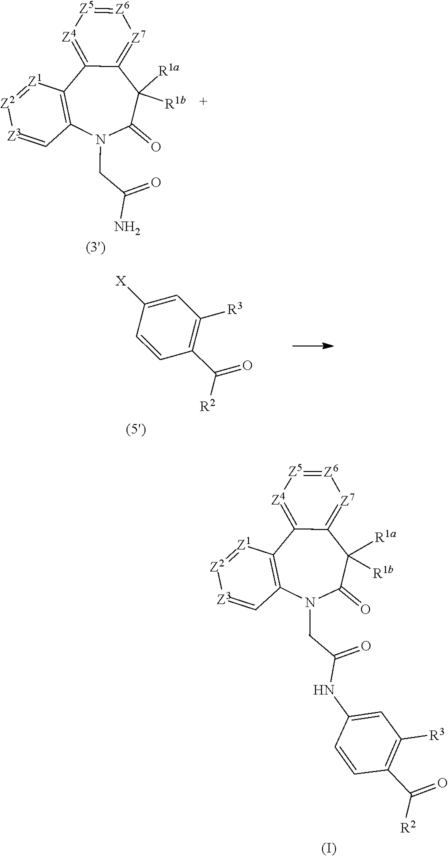

Compounds of Formula (5′) are either commercially available or maybe prepared by any method known to the person skilled in the art.

Compounds of Formula (3′) may be prepared by reaction of a compound of Formula (2) with an alpha-halogeno amide such as iodoacetamide in the presence of a base such as potassium carbonate in a polar solvent such as DMF at room temperature or by any other method known to the person skilled in the art. Alternatively, compounds of Formula (3′) may be prepared by reaction of a carboxylic acid or carboxylic acid derivatives of formula (3) with ammonia following procedures for the formation of an amide from carboxylic acids or carboxylic derivatives and amines known to the person skilled in the art.

Compounds of Formula (2) may be prepared by cyclocondensation from their precursors of Formula (6) in which LG² has the same definition as depicted above. For example, when LG² is an alkoxy, the reaction involves the presence of a base such as LiHMDS or K₂CO₃ or may be directly obtained without isolation from the previous step under heating conditions.

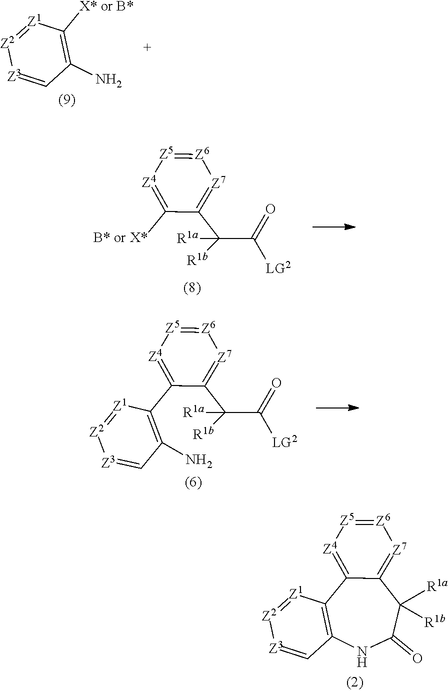

Compounds of Formula (6) may be prepared by a cross-coupling reaction, the “Suzuki reaction” known by the person skilled in the art, from their corresponding precursors of Formula (8) and (9), with the proviso that when (8) bears B*, (9) bears X* or when (8) bears X*, (9) bears B*. B* may be a boronic acid B(OH)₂, or any boronic ester B(OR)₂ such as pinacol boronic ester or a mixture of the two, and X* is a halogen such as Cl, Br or I.

Compounds of Formula (8) and (9) are either commercially available, described in the literature or may be prepared by functional group transformations known to the person skilled in the art.

Compounds of formula (I′) wherein A represents A² or A³ may be synthesized as described in the Examples.

In another aspect, the present invention provides synthetic intermediates of formula (II),

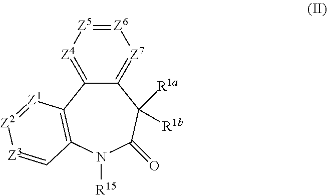

Wherein Z¹, Z², Z³, Z⁴, Z⁵, Z⁶, Z⁷, R¹³, R^{1b} are as defined hereabove:
R¹⁵ represents hydrogen or CH₂—CO—R^{d}; and
R^{d} represents hydroxy, halogen, amino or C₁₋₄ alkoxy.

In first embodiment, R¹⁵ represents CH₂—CO—R^{d}. In a second embodiment, R¹⁵ represents hydrogen.

In yet another aspect, the present invention relates to the use of intermediates of Formula (II) for the synthesis of compounds of formula (I).

### EXPERIMENTAL SECTION

### I. Abbreviations/Recurrent Reagents

ACN or MeCN Acetonitrile
CV Column Volume
DCM Dichloromethane
EtOAc Ethyl acetate
DMF N,N-Dimethylformamide
DMA Dimethyl acetamide
DMAP Dimethylaminopyridine
EDC 1-Ethyl-3-carbodiimide hydrochloride
MeOH Methanol
DCE Dichloroethane
HATU Hexafluorophosphate Azabenzotriazole Tetramethyl Uronium
HBTU Hexafluorophosphate Benzotriazole Tetramethyl Uronium
HOBt Hydroxybenzotriazole
TCFH Chloro-N,N,N′,N′-tetramethylformamidinium hexafluorophosphate
NMI N-Methylimidazole
MTBE or TBME Methyl tert-butyl ether
PTFE polytetrafluoroethylene
ELSD Evaporative light scattering detector
DMSO Dimethylsulfoxide
Brine Saturated aqueous sodium chloride solution
Et₂O Diethyl ether
h Hour
d Days
THE Tetrahydrofuran
AcOH Acetic acid
RT Room temperature
rt Retention time
Rf Retention factor
br Broad
M Molar
MS Mass Spectrometry
[M+H]⁺ Exact mass of protonated ion observed by MS
[M−H]⁻ Exact mass of deprotonated ion observed by MS
mL Milliliter
HPLC High Performance Liquid Chromatography
UPLC Ultra High Performance Liquid Chromatography
LC-MS Liquid Chromatography Mass Spectrometry
ESI Electrospray Ionisation
ES⁺ Electrospray Positive Ionisation
TEA Triethylamine
DIPEA N,N-di-iso-propylethylamine
DEA Diethylamine
CDI Carbonyl diimidazole
PCy3 Tricyclohexylphosphine
TMSCN Trimethylsilyl cyanide
dppf 1,1′-Bis(diphenylphosphino)ferrocene
PEPPSI Pyridine-Enhanced Precatalyst Preparation Stabilization and Initiation
HMDS bis(trimethylsilyl)amide or Hexamethyldisilazane
PPh₃ Triphenylphosphine
AIBN Azobisisobutyronitrile
TFA Trifluoroacetic acid
bs. Broad singlet
NBS N-bromosuccinimide
DME Dimethoxy ethane
HMPA Hexamethylphosphoramide
SFC Supercritical Fluid Chromatography
SCX Strong Cation Exchange HPLC column
TLC Thin Layer Chromatography
Sat. Saturated
Hex Hexane
aq. Aqueous
Eq. Equivalent
min Minute
mmol Millimole
UV Ultraviolet

### Naming Convention:

IUPAC names of chemical reagents, Intermediates and Examples have been generated using Biovia Draw 2020 (version 20.1.100.2161 or 20.1.0.2081). Depending on the Kekule structures of chemical reagents, Intermediates and Examples, Bovia Draw may generate different chemical names. As an illustration, the Kekule structures K1 and K2 are named 4,8,14-triazatricyclo[9.4.0.0^{2,7}]pentadeca-1(11),2,4,6,12,14-hexaen-9-one and 4,8,14-triazatricyclo[9.4.0.0^{2,7}]pentadeca-1(15),2,4,6,11,13-hexaen-9-one, respectively. Both names could be found in the below descriptions.

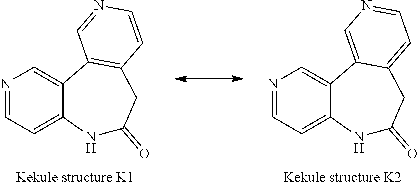 [SMILES: O=C1Cc2ccncc2-c2cnccc2N1 ; O=C1Cc2ccncc2-c2cnccc2N1]

### II. Analytical and Synthetic Methods

All reactions involving air or moisture-sensitive reagents are performed under a nitrogen or argon atmosphere (inert atmosphere) using dried solvents and glassware. Experiments requiring microwave irradiation are performed on a Biotage Initiator Sixty microwave oven upgraded with version 2.0 of the operating software. Experiments are run to reach the required temperature as quickly as possible (maximum irradiation power: 400 W, no external cooling). Commercial solvents and reagents are generally used without further purification, including anhydrous solvents when appropriate (generally Sure-Seal™ products from Aldrich Chemical Company or AcroSeal™ from ACROS Organics). In general, reactions are followed by thin layer chromatography (TLC), high performance liquid chromatography (HPLC) or mass spectrometry (MS) analyses.

NMR spectra were recorded on a Bruker Advance III HD 500 MHz or 400 MHz spectrometer. The chemical shifts (6) reported are given in parts per million (ppm), and the coupling constants (J) are in Hertz (Hz). The spin multiplicities are reported as s=singlet, bs=broad singlet, d=doublet, t=triplet, q=quartet, dd=doublet of doublet, ddd=doublet of doublet of doublet, dt=doublet of triplet, td=triplet of doublet, and m=multiplet.

### Mass Spectrometric Measurements in LC-MS Mode are Performed as Follows:

For acidic elution (Method A1, A1′, A1_S, A2 and A2′), analyses are performed using a QDA Waters simple quadrupole mass spectrometer. This spectrometer is equipped with an ESI source and an UPLC Acquity Hclass with diode array detector (200 to 400 nm). Data are acquired in a full MS scan from m/z 70 to 800 in positive mode with an acidic elution. The reverse phase separation is carried out at 45° C. on a Waters Acquity UPLC HSS T3 1.8 μm (2.1×50 mm) column for Method A1, A1′ and A1_S elution and on a Waters Acquity UPLC HSS T3 1.8 μm (2.1×100 mm) column for Method A2 and A2′. Gradient elution is done with water/ACN/TFA (95/5/0.5 mL/L) (solvent A) and ACN (solvent B) for Method A1, A1_S and A2 and Water/Acetonitrile/Formic acid (95/5/(0.05%)) (solvent A) Acetonitrile/Formic acid (99.95/0.05%) (solvent B) for Method A1′ and A2′. Injection volume: 1 μL. Full flow in MS. Gradient Program:

| | | | |
|---|---|---|---|
| Time | A | B | Flow |
| (min) | (%) | (%) | (mL/min) |

Method A1, A1′

| | | | |
|---|---|---|---|
| 0 | 99 | 1 | 0.4 |
| 0.3 | 99 | 1 | 0.4 |
| 3.2 | 5 | 95 | 0.4 |
| 3.25 | 5 | 95 | 0.5 |
| 4 | 5 | 95 | 0.5 |
| 4.1 | 99 | 1 | 0.4 |
| 5.5 | 99 | 1 | 0.4 |

Method A1_S

| | | | |
|---|---|---|---|
| 0 | 99 | 1 | 0.8 |
| 0.15 | 99 | 1 | 0.8 |
| 1.60 | 5 | 95 | 0.8 |
| 1.65 | 5 | 95 | 0.8 |
| 2 | 5 | 95 | 0.8 |
| 2.05 | 99 | 1 | 0.8 |
| 2.75 | 99 | 1 | 0.8 |

Method A2, A2′

| | | | |
|---|---|---|---|
| 0 | 99 | 1 | 0.4 |
| 0.8 | 99 | 1 | 0.4 |
| 5.3 | 5 | 95 | 0.4 |
| 5.35 | 5 | 95 | 0.5 |
| 7.3 | 5 | 95 | 0.5 |
| 7.35 | 99 | 1 | 0.4 |
| 9 | 99 | 1 | 0.4 |

For acidic elution (Method A3), analysis are performed using a Xevo Waters Q-TOF mass spectrometer. This spectrometer is equipped with an ESI source and a Waters Acquity H-class UPLC with diode array detector (210 to 400 nm). Data are acquired in a full MS scan from m/z 50 to 1200 in positive mode. The reverse phase separation is carried out at 40° C. on an Acquity UPLC HSS T3 C18 column (1.8 μm, 2.1×100 mm). Gradient elution is done with Water/ACN/Formic acid (95/5/750 μL/L) (Solvent C) and Water/ACN/Formic acid (5/95/500 μL/L) (Solvent D) at pH~3. 100% Flow in UV, 10% flow in MS-, 90% flow in ELSD. Injection volume: 0.5 to 2 μL.

### Gradient Program:

Method A3

| Time | C | D | Flow |
|---|---|---|---|
| (min) | (%) | (%) | (mL/min) |
| 0 | 98 | 2 | 0.6 |
| 0.3 | 98 | 2 | 0.6 |
| 5.9 | 5 | 95 | 0.6 |
| 9.3 | 5 | 95 | 0.6 |
| 9.4 | 98 | 2 | 0.6 |
| 14 | 98 | 2 | 0.6 |

For acidic elution (Method A4 and A8), analysis is performed using a SYNAPT G2-SI Waters Q-TOF mass spectrometer for Method A4 and on a SQD2 Waters single quadrupole for Method A8. These spectrometers are equipped with an ESI source and a Waters Acquity H-class UPLC with diode array detector (210 to 400 nm). Data are acquired in a full MS scan from m/z 50 to 1200 in positive mode. The reverse phase separation is carried out at 45° C. on an Acquity UPLC HSS T3 C18 column (1.8 μm, 2.1×100 mm). Gradient elution is done with Water/ACN/Formic acid (95/5/750 μL/L) (Solvent C) and Water/ACN/Formic acid (5/95/500 μL/L) (Solvent D) pH-3. Full flow in MS. injection volume: 0.5 μL.

### Gradient Program:

Method A4, A8

| Time | C | D | Flow |
|---|---|---|---|
| (min) | (%) | (%) | (mL/min) |
| 0 | 98 | 2 | 0.6 |
| 0.5 | 98 | 2 | 0.6 |
| 5 | 5 | 95 | 0.6 |
| 5.1 | 5 | 95 | 0.6 |
| 7.3 | 5 | 95 | 0.6 |
| 7.5 | 98 | 1 | 0.6 |
| 10 | 98 | 1 | 0.6 |

For acidic elution (Method A5), analyses are performed using a Shimadzu LC-MS 2010EV mass spectrometer for LC-MS analysis. This spectrometer is equipped with an ESI source and HPLC with diode array detector (210 to 400 nm). Data is acquired in a full MS scan from m/z 80 to 2000 in positive mode and negative mode. The reverse phase separation is carried out with Waters X-Select CSH C18 (4.6×150 mm), 3.5 μm column. Column temp: 50° C. Gradient elution is done with Mobile phase with 0.1% Formic acid in water (Phase A) and Acetonitrile (Phase B). Injection volume: 2 μL.

### Gradient Program:

Method A5

| Time | A | B | Flow |
|---|---|---|---|
| (min) | (%) | (%) | (mL/min) |
| 0.01 | 95 | 5 | 1 |
| 1.0 | 95 | 5 | 1 |
| 8.0 | 0 | 100 | 1 |
| 12 | 0 | 100 | 1 |
| 14 | 95 | 5 | 1 |
| 18 | 95 | 5 | 1 |

For acidic elution (Method A6), analyses are performed using a Shimadzu LC-MS 2010EV mass spectrometer for LC-MS analysis. This spectrometer is equipped with an ESI source and HPLC with diode array detector (210 to 400 nm). Data is acquired in a full MS scan from m/z 80 to 2000 in positive mode and negative mode. The reverse phase separation is carried out with Waters X-Select CSH C18 (4.6×150) mm, 3.5 μm column. Column temp: 50° C. Gradient elution is done with Mobile phase with 0.1% TFA in water (Phase A) and Acetonitrile (Phase B). Injection volume: 2 μL.

### Gradient Program:

Method A6

| Time | A | B | Flow |
|---|---|---|---|
| (min) | (%) | (%) | (mL/min) |
| 0.01 | 95 | 5 | 1.2 |
| 1.0 | 95 | 5 | 1.2 |
| 8.0 | 0 | 100 | 1.2 |
| 12 | 0 | 100 | 1.2 |
| 14 | 95 | 5 | 1.2 |
| 18 | 95 | 5 | 1.2 |

For acid elution (Method A7), analyses are performed using an Agilent 1200-6120 LC-MS system coupled to UV detection (254 nM) and MS Detection: Agilent 6120 Mass Spectrometer (ES) m/z 100 to 1000. Column: XSelect CSH C18 XP 130 Å, 2.5 μm, 4.6 mm×30 mm (Waters™). Mobile Phase A: 0.1% Formic acid in water, Mobile Phase B: Acetonitrile+0.1% Formic acid. Flow rate: 2.5 mL/min.

### Gradient Program:

Method A7

| Time | | | Flow |
|---|---|---|---|
| (min) | A(%) | B(%) | (mL/min) |
| 0 | 95 | 5 | 2.5 |
| 3 | 5 | 95 | 2.5 |
| 3.01 | 5 | 95 | 4.5 |
| 3.60 | 5 | 95 | 4.5 |
| 3.7 | 95 | 5 | 4.5 |
| 3.71 | 95 | 5 | 2.5 |
| 4 | 95 | 5 | 2.5 |

For acid elution (Method A9), analyses are performed using similar equipments as above, but the reverse phase separation is carried out with a Waters Cortecs C18 2.7 μm (30×2.1 mm) column. Column temp: 40° C. 1.5 min gradient elution is done with Mobile phase with 0.1% formic acid in water (Phase A) and ACN (Phase B).
For basic elution (Method B1, B1_S and B2), analyses are performed using a QDA Waters simple quadrupole mass spectrometer. This spectrometer is equipped with an ESI source and a UPLC Acquity Hclass with diode array detector (200 to 400 nm). Data are acquired in a full MS scan from m/z 70 to 800 in positive mode. The reverse phase separation is carried out at 45° C. on a Waters Acquity UPLC BEHC18 1.7 μm (2.1×50 mm) column for Method B1 and B1_S and on a Waters Acquity UPLC BEH C18 1.7 μm (2.1×100 mm) column basic elution for Method B2. Gradient elution is performed with water/ACN/ammonium formate (95/5/63 mg/L) (solvent A) and ACN/water/ammonium formate (95/5/63 mg/L) (solvent B). Injection volume: 1 μL. Full flow in MS.

### Gradient Program:

| | | | |
|---|---|---|---|
| Time | A | B | Flow |
| (min) | (%) | (%) | (mL/min) |

Method B1

| | | | |
|---|---|---|---|
| 0 | 99 | 1 | 0.4 |
| 0.3 | 99 | 1 | 0.4 |
| 3.2 | 0 | 100 | 0.4 |
| 3.25 | 0 | 100 | 0.5 |
| 4 | 0 | 100 | 0.5 |
| 4.1 | 99 | 1 | 0.4 |
| 4.8 | 90 | 1 | 0.4 |

Method B1_S

| | | | |
|---|---|---|---|
| 0 | 99 | 1 | 0.8 |
| 0.15 | 99 | 1 | 0.8 |
| 1.6 | 5 | 95 | 0.8 |
| 1.65 | 5 | 95 | 0.8 |
| 2 | 5 | 95 | 0.8 |
| 2.05 | 99 | 1 | 0.8 |
| 2.75 | 99 | 1 | 0.8 |

Method B2

| | | | |
|---|---|---|---|
| 0 | 99 | 1 | 0.4 |
| 0.8 | 99 | 1 | 0.4 |
| 5.30 | 0 | 100 | 0.4 |
| 5.35 | 0 | 100 | 0.5 |
| 7.30 | 0 | 100 | 0.5 |
| 7.35 | 99 | 1 | 0.4 |
| 9 | 90 | 1 | 0.4 |

For Basic elution (Method B3) analysis are performed using a Xevo Waters Q-TOF mass spectrometer. This spectrometer is equipped with an ESI source and a Waters Acquity H-class UPLC with diode array detector (210 to 400 nm). Data are acquired in a full MS scan from m/z 50 to 1200 in positive mode. The reverse phase separation is carried out at 45° C. on an Acquity UPLC BEH C18 column (1.7 μm, 2.1×100 mm). Gradient elution is done with Water/ACN/Ammonium formate (95/5/(40 mg/L ammonium bicarbonate+100 μL/L NH₄OH)) (Solvent A) and ACN (Solvent B) pH-8-9. 100% Flow in UV, 10% flow in MS. 90% Flow in ELSD injection volume: 0.2 to 2 μL.

### Gradient Program:

Method B3

| Time | A | B | Flow |
|---|---|---|---|
| (min) | (%) | (%) | (mL/min) |
| 0 | 98 | 2 | 0.6 |
| 0.3 | 98 | 2 | 0.6 |
| 5.9 | 5 | 95 | 0.6 |
| 9.3 | 5 | 95 | 0.6 |
| 9.4 | 98 | 2 | 0.6 |
| 14 | 98 | 2 | 0.6 |

For basic elution (Method B4 and B8) analysis are performed using a SYNAPT G2-SI system and Waters Q-TOF mass spectrometer for Method A4 and on a SQD2 Waters single quadrupole for Method B8. These spectrometer is equipped with an ESI source and a Waters Acquity H-class UPLC with diode array detector (210 to 400 nm). Data are acquired in a full MS scan from m/z 50 to 1200 in positive mode. The reverse phase separation is carried out at 45° C. on an Acquity UPLC BEH C18 column (1.7 μm, 2.1×100 mm). Gradient elution is done with water/ACN/ammonium formate (95/5/(63 mg/L+100 μL/L NH₄OH)) (solvent A) and ACN (solvent B), at pH~8-9. Full flow in MS. injection volume: 0.5 μL.

### Gradient Program:

Method B4, B8

| Time | | | Flow |
|---|---|---|---|
| (min) | A(%) | B(%) | (mL/min) |
| 0 | 98 | 2 | 0.6 |
| 0.5 | 98 | 2 | 0.6 |
| 5 | 5 | 95 | 0.6 |
| 5.5 | 5 | 95 | 0.7 |
| 7.3 | 5 | 95 | 0.7 |
| 7.5 | 98 | 1 | 0.6 |
| 10 | 98 | 1 | 0.6 |

For basic elution (Method B5 and B5′), analyses are performed using an Agilent 1200-6120 LC-MS system coupled to UV detection (254 nM) and a MS Detection Agilent 6120 Mass Spectrometer (ES) m/z 100 to 1000. The reverse phase separation is carried out at 45° C. on a XBridge BEH C18 XP Column, 130A, 2.5 μm, 4.6 mm×30 mm (Waters™). Column temp: 40° C. Flow rate: 2.5 mL/min. Gradient elution is done with Mobile phase Acetonitrile/10 mM aqueous ammonium bicarbonate (Phase A) and Acetonitrile (Phase B) for method B5 and Mobile Phase A: 0.1% Ammonia in water, Mobile Phase B: Acetonitrile for method B5′.

### Gradient Program:

Method B5, B5′

| Time | | | Flow |
|---|---|---|---|
| (min) | A(%) | B(%) | (mL/min) |
| 0 | 95 | 5 | 2.5 |
| 3 | 5 | 95 | 2.5 |
| 3.01 | 5 | 95 | 4.5 |
| 3.60 | 5 | 95 | 4.5 |
| 3.7 | 95 | 5 | 4.5 |
| 3.71 | 95 | 5 | 2.5 |
| 4 | 95 | 5 | 2.5 |

For Basic elution (Method B6), analyses are performed using a Shimadzu LC-MS 2010EV mass spectrometer. This spectrometer is equipped with an ESI source and HPLC with diode array detector (210 to 400 nm). Data is acquired in a full MS scan from m/z 80 to 2000 in positive mode and negative mode. The reverse phase separation is carried out with Waters X-Select CSH C18 (4.6×150) mm, 3.5 μm column. Column temp: 50° C. Gradient elution is done with Mobile phase: 10 mM ammonium bicarbonate in Water (Phase A) and acetonitrile (Phase B). Injection volume: 2 μL.

### Gradient Program:

Method B6

| Time | A | B | Flow |
|---|---|---|---|
| (min) | (%) | (%) | (mL/min) |
| 0.01 | 95 | 5 | 1 |
| 1.0 | 95 | 5 | 1 |
| 8.0 | 0 | 100 | 1 |
| 12 | 0 | 100 | 1 |
| 14 | 95 | 5 | 1 |
| 18 | 95 | 5 | 1 |

For Basic elution (Method B7), analyses are performed using a Shimadzu LC-MS 2010EV mass spectrometer. This spectrometer is equipped with an ESI source and HPLC with diode array detector (210 to 400 nm). Data is acquired in a full MS scan from m/z 80 to 2000 in positive mode and negative mode. The reverse phase separation is carried out with Waters X-Bridge C18 (4.6×150) mm, 5 μm column. Column temp: 50° C. Gradient elution is done with Mobile phase 0.1% Ammonia in Water (Phase A) and Acetonitrile (Phase B). Injection volume: 5 μL.

### Gradient Program:

Method B7

| Time | A | B | Flow |
|---|---|---|---|
| (min) | (%) | (%) | (mL/min) |
| 0.01 | 98 | 2 | 1.2 |
| 6 | 85 | 15 | 1.2 |
| 8 | 85 | 15 | 1.2 |
| 9 | 0 | 100 | 1.2 |
| 12 | 0 | 100 | 1.2 |
| 14 | 98 | 2 | 1.2 |
| 18 | 98 | 2 | 1.2 |

For Basic elution (Method B9), analyses are performed using an Agilent 1200 series LC in tandem with a 6140 mass spectrometer. The reverse phase separation is carried out with a Phenomenex Gemini NX-C18 3 μM (2×20 mm), flow rate 1.0 mL/min, column temperature 40° C., eluting with a 5-95% gradient over 6.0 minutes (solvent A: 10 mM ammonium formate in water+0.1% ammonia solution, solvent B: ACN+5% water+0.1% ammonia solution).
For Basic elution (Method B10), analyses are performed using an Agilent 1290 Infinity II LC in tandem with a 6135 MSD XT mass spectrometer. The reverse phase separation is carried out with an Acquity UPLC BEH C18 2.1×50 mm, 1.7 μM, flow rate 1.5 mL/min, 60° C. column temperature, eluting with a 5-95% gradient over 4.5 minutes (solvent A−10 mM ammonium formate in water+0.1% Ammonia solution, solvent B−ACN+5% water+0.1% ammonia solution).

Analytical chiral LC-MS were all performed at 30° C. on 4.6×150 mm columns with a flow rate of 1.5 mL/min except for Chiralpak IG-u (Daicel) column which dimension is 3×100 mm and the flow rate is 0.425 mL/min. All columns display a granulometry of 3 μm except for Whelk O-1 (R,R) (Regis Technology) which is 3.5 μm and Chiralpak IG-u (Daicel) which is sub-2 μm.

High Resolution Mass spectrometric measurements in LC-MS mode are performed as follows:

Method HRMS_A1: A SYNAPT G2-SI Waters Q-TOF mass spectrometer is used for QC analysis. This spectrometer is equipped with an ESI source and a Waters Acquity H-class UPLC with diode array detector (210 to 400 nm). Data are acquired in a full MS scan from m/z 50 to 1200 in positive mode. The reverse phase separation is carried out at 45° C. on an Acquity UPLC BEH C18 column (1.7 μm, 2.1×30 mm). Gradient elution is done with Water/ACN/Formic acid (95/5/(750 μL/L)) (Solvent C) and Water/ACN/Formic acid (5/95/(500 μL/L)) (Solvent D) at pH~3. Full flow in MS. injection volume: 0.5 to 1 μL.
Method HRMS_A2: analysis are performed using a Xevo Waters Q-TOF mass spectrometer. This spectrometer is equipped with an ESI source and a Waters Acquity H-class UPLC with diode array detector (210 to 400 nm). Data are acquired in a full MS scan from m/z 50 to 1200 in positive mode. The reverse phase separation is carried out at 40° C. on an Acquity UPLC HSS T3 C18 column (1.8 μm, 2.1×50 mm). Gradient elution is done with Water/ACN/Formic acid (95/5/750 μL/L) (Solvent C) and Water/ACN/Formic acid (5/95/500 μL/L) (Solvent D) at pH-3. 100% Flow in UV, 10% flow in MS-, 90% flow in ELSD. Injection volume: 0.5 to 1 μL.

| | | | |
|---|---|---|---|
| Time | C | D | Flow |
| (min) | (%) | (%) | (mL/min) |

Method HRMS_A1

| | | | |
|---|---|---|---|
| 0 | 95 | 5 | 0.8 |
| 1.8 | 5 | 95 | 0.8 |
| 2.4 | 5 | 95 | 0.8 |
| 2.5 | 95 | 5 | 0.8 |
| 3.1 | 95 | 5 | 0.8 |

Method HRMS_A2

| | | | |
|---|---|---|---|
| 0 | 98 | 2 | 0.8 |
| 0.3 | 98 | 2 | 0.8 |
| 3 | 5 | 95 | 0.8 |
| 4 | 5 | 95 | 0.8 |
| 4.1 | 98 | 2 | 0.8 |
| 5.1 | 98 | 2 | 0.8 |

Preparative HPLC purifications are performed using SQD Waters or QDa Performance single quadrupole mass spectrometer. This spectrometer is equipped with an ESI source, Waters 2525 binary pump coupled with 2767 sample Manager and with diode array detector (210 to 400 nm). Data are acquired in a full MS scan from m/z 100 to 850 in positive and negative modes. LC parameters: The reverse phase separation is carried out at room temperature on a Waters XBridge OBD MS C18 column (5 μm, 30×50 mm). Typical HPLC flow rate from 35 mL/min to 45 mL/min. Typical example of basic elution: gradient from solvent A (H₂O+10 mM NH₄HCO₃+50 μL/L NH₄OH) and solvent B (100% acetonitrile) [Purification Method P_B]. Typical example of acidic elution: gradient from solvent A (H₂O/TFA: 99.5%/0.5%) and solvent B (ACN/TFA: 99.5%/0.5%) [Purification Method P_A].

Some preparative HPLC purifications are performed using a Gilson Modular System (333 Prep-Scale HPLC Pump (Water), 334 Presp-Scale HPLC Pump (Acetonitrile), 334 Presp-Scale HPLC Pump (Modifier: a solution of 5 mL NH₄OH in 1000 mL of H₂O for basic elution [Purification Method G_B]. or a solution of 20 mL TFA in 1000 mL H₂O [Purification Method G_A]), 171 Diode Array Detector, GX-271 Prep Liquid Handler, PrepFC Fraction Collector) equipped with a YMC Triart-500 g-10 μm-76.5×200 mm column. Typical HPLC flow rate is 180 mL/min.

When analytical methods are not specified in the below protocols, the methods used were similar to the ones described above. It will be apparent to the person skilled in the art that there are analytical and preparative chromatographic methods analogues to the ones described above can be use for the below procedures.

### III. Intermediates

### Intermediate C1: N-(4-acetylphenyl)-2-chloro-acetamide

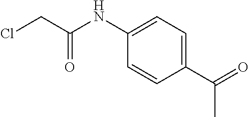

To a solution of 1-(4-aminophenyl)ethanone (2.0 g, 14.6 mmol) and triethylamine (2.5 mL, 18 mmol) in dry DCM (14.6 mL) was added dropwise at 0° C. chloroacetyl chloride (1.4 mL, 18 mmol). The resulting mixture was slowly warmed to room temperature and stirred at room temperature for 18 h. The reaction mixture was quenched by addition of water and extracted with DCM The combined organic extracts were washed with brine, dried over MgSO₄, filtered, and concentrated under vacuum. The residue was purified by flash chromatography on silica gel (using a gradient of 0 to 100% EtOAc in Heptane as eluent) to afford the title compound (2.65 g, yield: 86%) as a beige solid. LC-MS (Method B1_S) m/z: [M+H]⁺: 212.0; rt: 0.93 min; purity: 100%. ¹H NMR (400 MHz, DMSO-d₆) δ 10.61 (s, 1H), 7.95 (d, J=8.8 Hz, 2H), 7.72 (d, J=8.8 Hz, 2H), 4.30 (s, 2H), 2.53 (s, 3H).

### Intermediate C2: N-(4-acetyl-3-hydroxy-phenyl)-2-chloro-acetamide

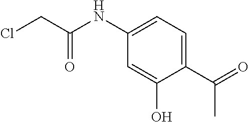

To a solution of 1-(4-amino-2-hydroxyphenyl)ethanone (200 mg, 1.32 mmol) and DIPEA (485 μL, 2.92 mmol) in dry DCM (4.00 mL) was added dropwise chloroacetyl chloride (172 μL, 2.12 mmol) under N₂ atmosphere. The resulting mixture was stirred at room temperature for 16 h. Extra chloroacetyl chloride (40 μL, 0.49 mmol) was added and the reaction mixture was further stirred at room temperature for 4 h. After completion, the reaction mixture was diluted with DCM (50 mL) and washed with water (2×50 mL). The organic layer was dried over MgSO₄, filtered and concentrated under vacuum. The residue was purified by flash chromatography on silica gel (using a gradient of 0 to 100% EtOAc in Heptane as eluent) to afford the title compound as a brownish solid (163 mg, yield: 54%). LC-MS (Method A1_S) m/z: [M+H]⁺:227.9; rt: 1.09 min; purity: 99%. ¹H NMR (400 MHz, DMSO-d₆) δ 12.27 (s, 1H), 10.61 (s, 1H), 7.88 (d, J=8.7 Hz, 1H), 7.34 (d, J=2.0 Hz, 1H), 7.08 (dd, J=8.7, 2.0 Hz, 1H), 4.30 (s, 2H), 2.58 (s, 3H).

### Intermediate L1: 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic, trifluoroacetic acid salt

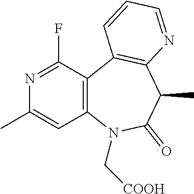

### Step 1: Synthesis of dimethyl 2-(4-amino-3,5-dichloro-6-fluoro-2-pyridyl)propanedioate L1_1

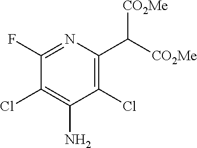 [SMILES: COC(=O)C(C(=O)OC)c1nc(F)c(Cl)c(N)c1Cl]

To a mixture of 4-amino-3,5-dichloro-2,6-difluoropyridine (402.2 g, 2021 mmol), dimethyl malonate (470 mL, 4030 mmol) and N,N-dimethylformamide (2.4 L) (colorless solution) was added potassium carbonate (830 g, 6005 mmol) (white suspension). The mixture was heated to 65° C. (internal temperature) for 19 hours. The mixture was cooled to ambient temperature and poured into mechanically stirred water (6 L). The solid was recovered on a sinter, washed with water (2 L) and dried. Trituration with TBME (2 L) and washing with isohexane (1.5 L) gave the title product (529 g, 84%) as a white crystalline solid. ¹H NMR (300 MHz, DMSO) δ 7.24 (s, 2H), 5.20 (s, 1H), 3.71 (s, 6H). ¹⁹F NMR (282 MHz, DMSO) δ −75.10. LC/MS (method B9) m/z (ES): 310.8, 312.8, 314.8 (M+H).

### Step 2: Synthesis of 3,5-dichloro-2-fluoro-6-methyl-pyridin-4-amine L1_2

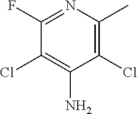

To dimethyl 2-(4-amino-3,5-dichloro-6-fluoro-2-pyridyl)propanedioate (Intermediate L1_1, 370 g, 1190 mmol) and calcium chloride hexahydrate (780 g, 3560 mmol) was added 1-methyl-2-pyrrolidinone (1.5 L). The suspension was heated to 100° C. to give a solution. After 20 hours, analysis by LCMS showed 87% conversion to mono-ester. The temperature was increased to 125° C. After 72 hours, analysis by LCMS showed complete conversion. The mixture was cooled to 25° C. and poured into mechanically stirred water (2.5 L). The precipitate was recovered on a sinter, washed with water (1 L) and partially dried. The wet solid was suspended in ethanol (3 L) and heated to 65° C. to give a cloudy solution. Activated charcoal (30 g) was added and heating continued. After 15 minutes, the warm mixture was filtered through Celite. To the filtrate was added water (3 L, to give 1:1 ethanol:water). The mixture was then cooled to 5° C. for 20 hours. The crystals were recovered on a sinter, washed with water (600 mL) and dried (Note: solid is slightly volatile if dried at 50° C. at ~2 mbar) to give the title product as an off-white solid (179 g, 77%). ¹H NMR (300 MHz, DMSO) δ 6.94 (s, 2H), 2.35 (d, J=0.6 Hz, 3H). ¹⁹F NMR (282 MHz, DMSO) δ −76.01. LC/MS (method B9) m/z (ES): 194.8, 196.8, 198.8 (M+H), rt: 1.38 min, 100% purity.

### Step 3: Preparation of 2-fluoro-6-methyl-pyridin-4-amine L1_3

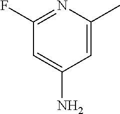

3,5-dichloro-2-fluoro-6-methyl-pyridin-4-amine (Intermediate L1_2, 150 g, 769 mmol) and 5% palladium on charcoal (49 g, 23 mmol) were solubilized in methanol (2.6 L) and pyridine (125 mL). The mixture was placed under a 1 bar hydrogen atmosphere and heated to 50° C. for 18 hours. The mixture was then cooled, filtered through Celite and concentrated under reduced pressure. Water (750 mL) was added to the residue and the solution was extracted with ethyl acetate (3×750 mL). The organics were washed with brine (500 mL), dried over MgSO₄ filtered and concentrated under reduced pressure to give the title product as a white solid (93 g, 91%). ¹H NMR (400 MHz, DMSO) δ 6.30 (s, 2H), 6.24 (s, 1H), 5.85 (s, 1H), 2.17 (t, J=1.5 Hz, 3H). ¹⁹F NMR (376 MHz, DMSO) δ −71.85. LC/MS (method B2) m/z (ES): 127.0 (M+H), rt: 2.1 min, 98.6% purity.

### Step 4: Synthesis of 2-fluoro-6-methyl-3-(4,4,5,5-tetramethyl-1,3,2-dioxaborolan-2-yl)pyridin-4-amine L1_4

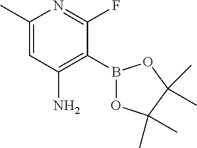

In a nitrogen filled glovebox, 2-fluoro-6-methyl-pyridin-4-amine (Intermediate L1_3, 138 mg, 1.04 mmol) was dissolved in dry THE (1 mL) in a 6 mL pressure tube containing a magnetic stir bar. Pinacolborane (211 μL, 1.45 mmol) was added and the reaction mixture was stirred at room temperature for 1 h. (1,5-Cyclooctadiene)(methoxy)iridium(I) dimer (10 mg, 0.015 mmol), 4,4′-di-tert-butyl-2,2′-dipyridyl (9 mg, 0.033 mmol) and bis(pinacolato)diboron (158 mg, 0.62 mmol) were added, the tube was sealed under nitrogen atmosphere and the reaction mixture was heated at 80° C. for 16 h. After cooling to room temperature, methanol (3 mL) was added and the reaction mixture was stirred for 10 min until the gas evolution has ceased, before being concentrated under vacuum. The crude brown oil was purified by column chromatography on silica gel (using a gradient DCM/EtOAc from 100/0 to 50/50 as eluent) and by trituration in hexane to afford the title compound as light pink solid (200 mg, yield: 72%). LC-MS (Method B1) m/z: [M+H]⁺: 253.0, rt: 1.13 min, purity: 95%. ¹H NMR (400 MHz, DMSO-d₆) δ 6.58 (s, 2H), 6.29 (d, J=1.9 Hz, 1H), 2.16 (s, 3H), 1.28 (s, 12H).

### Step 5: Synthesis of methyl 2-[3-(4-amino-2-fluoro-6-methyl-3-pyridyl)-2-pyridyl]propanoate L1_5

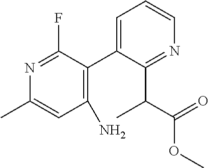 [SMILES: COC(=O)C(C)c1ncccc1-c1c(N)cc(C)nc1F]

To a solution of 2-fluoro-6-methyl-3-(4,4,5,5-tetramethyl-1,3,2-dioxaborolan-2-yl)pyridin-4-amine (Intermediate L1_4, 100 mg, 0.39 mmol) and methyl 2-(3-bromopyridin-2-yl)propanoate (102 mg, 0.40 mmol) in dry toluene (2 mL) was added potassium carbonate (170 mg, 1.22 mmol). Argon was passed throughout the reaction mixture before addition of tris(dibenzylideneacetone)dipalladium(0) (36 mg, 0.04 mmol) and 2-dicyclohexylphosphino-2,6-dimethoxybiphenyl (17 mg, 0.04 mmol). The reaction mixture was then stirred at 100° C. for 4 h. After cooling to room temperature, the reaction mixture was diluted with EtOAc (10 mL) and filtered through a PTFE filter. The filtrate was concentrated under vacuum to afford the crude title compound as a yellow oil (225 mg) which was directly used in the next step without purification. LC-MS (Method A1) m/z: [M+H]⁺: 290; rt: 0.80, 0.84 and 0.92 min. (as mixture of diastereoisomers).

### Step 6: Synthesis of 3-fluoro-5,10-dimethyl-4,8,12-triazatricyclo[9.4.0.0^{2,7}]pentadeca-1(11),2(7),3,5,12,14-hexaen-9-one L1_6

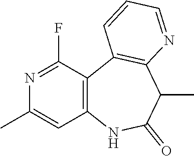

At 0° C., to a solution of crude methyl 2-[3-(4-amino-2-fluoro-6-methyl-3-pyridyl)-2-pyridyl]propanoate (Intermediate L1_5, 115 mg, 0.40 mmol) in dry toluene (2 mL) was added a 1.5 M solution of lithium bis(trimethylsilyl)amide in THE (200 μL, 0.30 mmol) and the resulting mixture was stirred at room temperature for 30 min. The reaction mixture was neutralized at 0° C. by the addition of saturated aqueous NH₄Cl and extracted with EtOAc (3×20 mL). The combined organic layers were washed with brine (20 mL), dried over MgSO₄, filtered off and concentrated under vacuum to give a yellow solid. Purification by trituration in Et₂O afforded the title compound as a brown solid (11 mg, yield: 10%). LC-MS (Method A1) m/z: [M+H]⁺: 258.0; rt: 0.93 min; purity: 94%.

### Step 7: Synthesis of tert-butyl 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetate L1_7

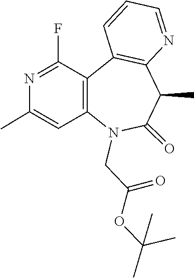

To a solution of intermediate L1_6 (1.05 g, 3.47 mmol) in dry DMF (17 mL) were added tert-butyl bromoacetate (627 μL, 4.16 mmol), potassium carbonate (970 mg, 6.90 mmol) and potassium iodide (58 mg, 0.35 mmol) and the resulting mixture was stirred at room temperature for 20 h. Water was added and the reaction mixture was extracted with EtOAc. The combined organic layers were washed with brine, dried over Na₂SO₄, filtered off and concentrated to dryness. The crude orange oil was purified by flash chromatography on silica gel (using a gradient of heptane/EtOAc from 100/0 to 0/100) to afford the title compound (1.22 g, yield: 92%) as a yellow solid. LC-MS (Method-A1) m/z: [M+H]⁺: 372.4, rt: 1.33 min, purity: 97%. ¹H NMR (400 MHz, DMSO-d₆) δ 8.66 (dd, J=4.8, 1.6 Hz, 1H), 8.11 (ddd, J=7.9, 4.8, 1.6 Hz, 1H), 7.48 (dd, J=7.9, 4.8 Hz, 1H), 7.29 (s, 1H), 4.65-4.30 (m, 2H), 3.74 (d, J=6.6 Hz, 1H), 2.52 (s, 3H), 1.49 (d, J=6.6 Hz, 3H), 1.24 (s, 9H). The racemate (21.5 g) was separated by Chiral SFC (Whelk O-1 (R,R) from Regis Technology, CO₂+iPrOH 20%) to afford the title compound (9.22 g, yield: 45%). Chiral purity: 99%; rt=2.52 min (first eluting enantiomer). For information, second eluting enantiomer rt=3.27 min. Both measured by HPLC (Whelk O-1 (R,R) from Regis Technology, iPrOH 50%−heptane 50%−DEA 0.1%).

### Step 8: 2-[(10R)-3-fluoro-5,10-dimethyl-9-oxo-4,8,12-triazatricyclo[9.4.0.0^{2,7}]pentadeca-1(11),2(7),3,5,12,14-hexaen-8-yl]acetic acid, trifluoroacetic acid salt L1

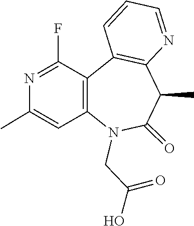

To a solution of Intermediate L1_7 (2.28 g, 5.89 mmol) in DCM (18 mL) was added at room temperature TFA (18 mL). The resulting mixture was stirred at room temperature for 2 h. After completion, the reaction mixture was concentrated to dryness and co-evaporated with Et₂O to afford the title compound as white solid (TFA salt, 2.85 g, yield: 94%). LC-MS (Method A1_S) m/z: [M+H]⁺: 316.1; rt: 0.92 min; purity: 99%. ¹H NMR (400 MHz, DMSO-d₆) δ 8.66 (dd, J=4.8, 1.6 Hz, 1H), 8.13-8.06 (m, 1H), 7.48 (dd, J=7.9, 4.8 Hz, 1H), 7.31 (s, 1H), 4.44 (s, 2H), 3.76 (q, J=6.6 Hz, 1H), 1.49 (d, J=6.6 Hz, 3H). Proton for COOH not observed. CH₃ protons under DMSO signal. ¹⁹F NMR (376 MHz, DMSO-d₆) δ −70.82 (d, J=4.7 Hz). Chiral purity: 100%; rt=1.80 min (first eluting enantiomer). For information, second eluting enantiomer rt=2.91 min. Both measured by HPLC (Chiralpak AD from Daicel, EtOH 30%−heptane 70%−DEA 0.1%).

### Intermediate L2: 3,14-difluoro-5,10-dimethyl-4,8,12-triazatricyclo[9.4.0.0^{2,7}]pentadeca-1(11),2(7),3,5,12,14-hexaen-9-one

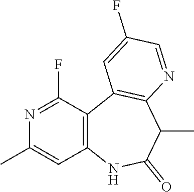

### Step 1: Synthesis of O1-tert-butyl O3-ethyl 2-(3-bromo-5-fluoro-2-pyridyl)propanedioate L2_1

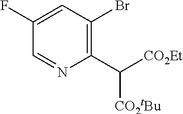 [SMILES: CCOC(=O)C(C(=O)OC(C)(C)C)c1ncc(F)cc1Br]

3-Bromo-2,5-difluoropyridine (163.3 g, 800 mmol) and dimethyl sulfoxide (3.2 L) were charged into a 5 L Process reactor. To the stirred solution was added tert-butyl ethyl malonate (300 mL, 1610 mmol) followed by Cs₂CO₃ (652 g, 2000 mmol) at rt. The resulting reaction mixture was stirred at 100° C. (internal temperature) for 21 hours before it was cooled down to rt, quenched with ice-cold water (1.7 L), and extracted with tert-butyl methyl ether (4×1.5 L). Combined organic extracts were washed with water (2×1.5 L), brine (2×1.2 L), dried over sodium sulfate, filtered and concentrated under vacuo to give the crude product (266 g) as a tan residue, which was then purified by flash chromatography (silica, gradient from 0 to 15% ethyl acetate in iso-hexane) to afford O1-tert-butyl 03-ethyl 2-(3-bromo-5-fluoro-2-pyridyl)propanedioate (120.6 g, 330 mmol, 42% Yield) as a white solid that spontaneously precipitated from chromatographed fractions. The precipitate was filtered off and the filtrates were concentrated and the resulting yellow residue (67 g) further purified by chromatography (silica, gradient from 0 to 10% ethyl acetate in hexane). The resulting pale yellow solid was slurried in ethyl acetate (20 mL) and iso-hexane (20 mL) and filtered off. The solid was washed with iso-hexane (2×20 mL) to afford O1-tert-butyl O3-ethyl 2-(3-bromo-5-fluoro-2-pyridyl)propanedioate (22.1 g, 61.0 mmol, 7.6% Yield). LC-MS (method B10) m/z: 306.0/308.0 (M-tBu+H), 262.0/264.0 (M-Boc+H); rt: 2.1 min, purity: 100%. ¹H NMR (300 MHz, CDCl₃) δ 8.41 (d, J=2.6 Hz, 1H), 7.66 (dd, J=7.5, 2.6 Hz, 1H), 5.10 (s, 1H), 4.29 (qd, J=7.2, 4.5 Hz, 2H), 1.49 (s, 9H), 1.29 (t, J=7.1 Hz, 3H). ¹⁹F NMR (282 MHz, CDCl₃) δ −125.48 (d, J=7.4 Hz). ¹³C NMR (101 MHz, CDCl₃) δ 166.98, 165.57, 157.80 (d, J=263.2 Hz), 149.25 (d, J=4.0 Hz), 136.19 (d, J=23.1 Hz), 127.43 (d, J=20.8 Hz), 121.16 (d, J=3.5 Hz), 82.90, 61.97, 60.01, 27.88, 14.06.

### Step 2: Synthesis of ethyl 2-(3-bromo-5-fluoro-2-pyridyl)acetate L2_2

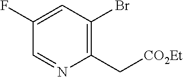 [SMILES: CCOC(=O)Cc1ncc(F)cc1Br]

To a 2 L Process Reactor were added dichloromethane (400 mL) and trifluoroacetic acid (300 mL). To the stirred solution was added a freshly prepared solution of O1-tert-butyl O3-ethyl 2-(3-bromo-5-fluoro-2-pyridyl)propanedioate L2_1 (69.3 g, 191 mmol) in dichloromethane (400 mL) at rt. The reaction mixture was stirred at rt for 5 hours before it was quenched by pouring into two stirred beakers of ice-water (2×1.7 L). The stirred mixture was carefully neutralised with solid K₃PO₄ to pH 7. The mixtures were then partitioned and organic extracts combined. The separated aqueous layer was extracted with dichloromethane (2×1 L). The combined organic extracts were washed with saturated NaHCO₃ solution (1.5 L), dried over sodium sulfate and concentrated under vacuo to give ethyl 2-(3-bromo-5-fluoro-2-pyridyl)acetate (53.6 g, 200 mmol, 100% Yield) as a yellow oil. LC-MS (method B10) m/z: 262.0/264.0 [M+H]⁺; rt: 1.42 min, purity: 100%. ¹H NMR (300 MHz, CDCl₃) δ 8.39 (d, J=2.6 Hz, 1H), 7.66 (dd, J=7.5, 2.6 Hz, 1H), 4.20 (q, J=7.1 Hz, 2H), 4.02 (s, 2H), 1.27 (t, J=7.1 Hz, 3H). ¹⁹F NMR (282 MHz, CDCl₃) δ −126.48 (d, J=7.6 Hz). ¹³C NMR (101 MHz, CDCl₃) δ 169.43, 157.64 (d, J=262.2 Hz), 149.91, 136.18 (d, J=22.6 Hz), 127.39 (d, J=20.6 Hz), 121.11, 61.27, 43.00, 14.16.

### Step 3: Synthesis of ethyl 2-(3-bromo-5-fluoro-2-pyridyl)propanoate L2_3

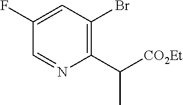

To a stirred solution of lithium diisopropylamide (2.0 M in THF/heptane/ethylbenzene, 56 mL, 110 mmol) in anhydrous tetrahydrofuran (340 mL) under nitrogen at −74° C. was added a solution of ethyl 2-(3-bromo-5-fluoro-2-pyridyl)acetate L2_2 (28.3 g, 100 mmol) in anhydrous tetrahydrofuran (100 mL) dropwise via a dropping funnel maintaining the internal temperature below −70° C. The resulting dark brown mixture was stirred for 30 minutes at −72° C. before iodomethane (9.6 mL, 150 mmol) was added dropwise. Stirring at −72° C. was continued for 30 minutes before the cooling bath was removed and the reaction mixture was allowed to warm up to rt. After 16 hours the reaction mixture was quenched with saturated NH₄Cl solution (450 mL) and extracted with ethyl acetate (2×450 mL). The combined organic extracts were washed with water (450 mL) and brine (300 mL), dried over sodium sulfate and concentrated under vacuo. Purification by chromatography (silica, gradient from 0 to 5% ethyl acetate in iso-hexane) afforded ethyl 2-(3-bromo-5-fluoro-2-pyridyl)propanoate (25.64 g, 92.86 mmol, 91% Yield) as a yellow oil. LC-MS (method B10) m/z: 276.0/278.0 [M+H]⁺; rt: 1.74 min, purity: 100%. ¹H NMR (300 MHz, CDCl₃) δ 8.40 (d, J=2.6 Hz, 1H), 7.64 (dd, J=7.6, 2.6 Hz, 1H), 4.33 (q, J=7.1 Hz, 1H), 4.16 (q, J=7.1 Hz, 2H), 1.53 (d, J=7.1 Hz, 3H), 1.20 (t, J=7.1 Hz, 3H). ¹⁹F NMR (282 MHz, CDCl₃) δ −126.94 (d, J=7.6 Hz).

### Step 4: Synthesis of 3,14-difluoro-5,10-dimethyl-4,8,12-triazatricyclo[9.4.0.0′⁷]pentadeca-1(11),2(7),3,5,12,14-hexaen-9-one L2

Under argon atmosphere, to a mixture of ethyl 2-(3-bromo-5-fluoro-2-pyridyl)propanoate (Intermediate L2_3, 2.00 g, 7.24 mmol), 2-fluoro-6-methyl-3-(4,4,5,5-tetramethyl-1,3,2-dioxaborolan-2-yl)pyridin-4-amine (Intermediate L1_4, 2.19 g, 8.69 mmol) and K₃PO₄ (3.17 g, 14.5 mmol) in dry Toluene (36 mL) at room temperature was added Tris(dibenzylideneacetone)dipalladium(0) (663 mg, 0.72 mmol) followed by 2-Dicyclohexylphosphino-2,6-dimethoxybiphenyl (303 mg, 0.72 mmol). The resulting mixture was stirred at 100° C. for 2 h. After completion, the reaction mixture was diluted with EtOAc (100 mL) and filtered over Celite. The filtrate was concentrated to dryness. The residue was dissolved in dry Toluene (36 mL) before addition, at room temperature, of LiHMDS (1.5 M in THF, 9.70 mL, 14.5 mmol). The resulting mixture was stirred at room temperature for 1 h. After complete conversion, the reaction mixture was quenched with water (80 mL) and extracted with EtOAc (4×20 mL). The combined organic extracts were washed with brine, dried over MgSO₄, filtered and concentrated under vacuum. The residue was purified by flash chromatography on silica gel (using a gradient of 20 to 100% EtOAc in Heptane as eluent) then triturated in Et₂O (20 mL), filtered and vacuum-dried to afford the title compound as a beige solid (634 mg, yield: 32%). LC-MS (Method A1_S) m/z: [M+H]⁺: 276.0; rt: 1.09 min; purity: 100%. ¹⁹F NMR (376 MHz, DMSO-d₆) δ −70.19 (d, J=4.1 Hz), −130.44 (d, J=9.8 Hz).

The racemate intermediate L2 (74.8 g) was separated by Chiral SFC (Chiralpak IG from Daicel, CO₂+MeOH 35%). The desired enantiomer (first eluting product) was triturated in iPrOH (300 mL) at 45° C., filtered, rinsed with iPrOH (30 mL) and dried under high vacuum at 50° C. for 2 h to afford the title compound (34.3 g, yield: 46%) as a white solid. LC-MS (Method A2) m/z: [M+H]⁺: 275.9; rt: 3.71 min; purity: 100%. LC-MS (Method B2) m/z: [M+H]⁺: 275.9 rt: 3.61 min; purity: 100%. ¹H NMR (500 MHz, DMSO-d₆) δ 10.87 (s, 1H), 8.69 (s, 1H), 8.07 (d, J=9.5 Hz, 1H), 7.01 (s, 1H), 3.64 (t, J=6.5 Hz, 1H), 2.46 (s, 3H), 1.48 (d, J=6.5 Hz, 3H). Chiral purity: 100%; rt=1.87 min (first eluting enantiomer). For information, second eluting enantiomer rt=2.52 min. Both measured by HPLC (Chiralpak IG from Daicel, MeOH 100%−DEA 0.1%).

### Intermediate L3: 14-fluoro-5,10-dimethyl-4,8,12-triazatricyclo[9.4.0.0^{2,7}]pentadeca-1(11),2,4,6,12,14-hexaen-9-one

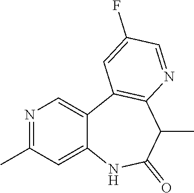

In a 60 mL vial, to a solution of ethyl 2-(3-bromo-5-fluoro-2-pyridyl)propanoate (Intermediate L2_3, 1 g, 3.62 mmol) in 1,4-dioxane (14.5 mL) at room temperature were added 2-methyl-5-(tetramethyl-1,3,2-dioxaborolan-2-yl)pyridin-4-amine (CAS: 1668475-78-2, 3.56 g, 5.43 mmol, 1.5 eq.), potassium carbonate (1.52 g, 10.9 mmol, 3.01 eq.) and water (3.6 mL). The mixture was evacuated and back-filled with N₂ (3×) before addition of XPHOS Pd G3 (161 mg, 0.18 mmol, 0.05 eq.) and X-PHOS (88 mg, 0.18 mmol, 0.05 eq.). The resulting mixture was evacuated, back-filled with N₂ (3 x) and then stirred at 100° C. for 3 h. The reaction mixture was diluted with EtOAc and water, filtered over a pad of celite and rinsed with EtOAc. The phases were separated and the aqueous layer was extracted two times with EtOAc. The combined organic layers were washed with brine, dried over Na₂SO₄, filtered and concentrated to dryness affording the crude mixture as an orange solid which was used in the next step without further purification. The residue (1.1 g) was dissolved in dry THE (18 mL) before addition, at 0° C., of LiHMDS (1.5 M in THF, 4.8 mL, 7.2 mmol). The resulting mixture was stirred at room temperature for 1 h. After complete conversion, the reaction mixture was diluted with methanol (5 mL), celite was added and the residue was concentrated to dryness and purified by flash chromatography on silica gel (using a gradient of 0 to 10% MeOH in DCM as eluent) to afford the title compound as a pale yellow solid (842 mg, yield: 90%). LC-MS (Method A1_S) m/z: [M+H]⁺: 258; rt: 0.70 min; purity: 100%. ¹H NMR (400 MHz, DMSO-d₆) δ 10.66 (s, 1H), 8.76 (s, 1H), 8.67 (d, J=2.8 Hz, 1H), 8.12 (dd, J=9.7, 2.8 Hz, 1H), 7.03 (s, 1H), 3.51 (q, J=6.6 Hz, 1H), 1.48 (d, J=6.6 Hz, 3H). CHs protons under DMSO signal.

### Intermediate L4: (10R)-3-chloro-14-fluoro-5,10-dimethyl-4,8,12-triazatricyclo[9.4.0.0^{2,7}]pentadeca-1(11),2(7),3,5,12,14-hexaen-9-one

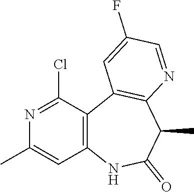

### Step 1: Synthesis of 3-bromo-2-chloro-6-methyl-pyridin-4-amine L4_1

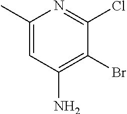

A solution of 2-chloro-6-methyl-pyridin-4-amine (10.0 g, 66.6 mmol) in dry acetonitrile (300 mL) was cooled to 0° C., then N-bromosuccinimide (11.3 g, 63.3 mmol) was added over 1 h and the reaction mixture was stirred at 0° C. for 3 h, then warmed to room temperature and stirred for a further 18 h. The reaction mixture was then concentrated under vacuum and purified by column chromatography on silica gel (using a gradient of 0 to 50% EtOAc in iso-hexane as eluent) to afford the title compound (7.05 g, yield: 47%) as a yellow solid. ¹H NMR (400 MHz, DMSO-d₆) δ 6.53 (s, 2H), 6.47 (d, J=0.7 Hz, 1H), 2.20 (s, 3H).

### Step 2: Synthesis of ethyl 2-[5-fluoro-3-(4,4,5,5-tetramethyl-1,3,2-dioxaborolan-2-yl)-2-pyridyl]propanoate L4_2

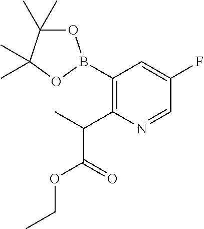 [SMILES: CCOC(=O)C(C)c1ncc(F)cc1B1OC(C)(C)C(C)(C)O1]

To a nitrogen sparged suspension of ethyl 2-(3-bromo-5-fluoro-2-pyridyl)propanoate L2_3 (86% purity, 1.50 g, 4.67 mmol), 4,4,5,5-tetramethyl-2-(4,4,5,5-tetramethyl-1,3,2-dioxaborolan-2-yl)-1,3,2-dioxaborolane (1.4 g, 5.61 mmol) and CH₃COOK (1.8 g, 18.7 mmol) in 1,4-dioxane (13 mL) was added [1,1′-bis(diphenylphosphino)ferrocene]dichloropalladium(II) DCM complex (172 mg, 0.21 mmol) and the reaction mixture was sparged with nitrogen for a further 10 min. The suspension was heated at reflux for 4 h. The reaction mixture was allowed to cool to room temperature then filtered over celite and washed through with EtOAc (100 mL). The filtrate was concentrated under reduced pressure then purified by column chromatography on silica gel (using a gradient of 0-100% EtOAc in isohexane as eluent) to afford the title compound as a clear colourless oil which partially crystallised upon standing (950 mg, yield: 35%). LC-MS (Method A9) m/z [M+H]P: 324.4; rt: 1.27 min, purity: 55%. ¹H NMR (400 MHz, CDCl₃) δ 8.42 (d, J=3.1 Hz, 1H), 7.77 (dd, J=8.8, 3.1 Hz, 1H), 4.67 (q, J=7.0 Hz, 1H), 4.17-4.09 (m, 2H), 1.50 (d, J=7.1 Hz, 3H), 1.35 (s, 12H), 1.18 (t, J=7.2 Hz, 3H).

### Step 3: Synthesis of (10R)-3-chloro-14-fluoro-5,10-dimethyl-4,8,12-triazatricyclo[9.4.0.0^{2,7}]pentadeca-1(11),2(7),3,5,12,14-hexaen-9-one L4

To a solution of 3-bromo-2-chloro-6-methyl-pyridin-4-amine (Intermediate L4_1, 860 mg, 3.87 mmol) and ethyl 2-[5-fluoro-3-(4,4,5,5-tetramethyl-1,3,2-dioxaborolan-2-yl)-2-pyridyl]propanoate (Intermediate L4_2, 2.09 g, 4.46 mmol) in 1,4-dioxane (31 mL) was added CsF (5 M in water, 1.94 mL, 9.70 mmol) followed by bis(di-tert-butyl(4-dimethylaminophenyl)phosphine)dichloropalladium(II) (140 mg, 0.19 mmol). The resulting mixture was stirred at 85° C. for 16 h. After completion, the reaction mixture was filtered over a pad of celite and rinsed with EtOAc. The filtrate was concentrated to dryness. The residue was dissolved in dry Toluene (20 mL) before addition of LiHMDS (1.5 M in THF, 7.70 mL, 11.6 mmol) at room temperature. The resulting mixture was stirred at room temperature for 2 h. After completion, the reaction mixture was concentrated under vacuum. The residue was purified twice by flash chromatography on silica gel (first by using a gradient of 0 to 5% MeOH in DCM, second by using a gradient of 0 to 70% EtOAc in Heptane as eluent) to afford the title compound as a white solid (333 mg, yield: 30%). LC-MS (Method A1_S) m/z: [M+H]⁺: 292; rt: 1.16 min; purity: 100%. ¹H NMR (400 MHz, DMSO-d₆) δ 10.74 (s, 1H), 8.68 (d, J=2.8 Hz, 1H), 8.19 (dd, J=9.9, 2.8 Hz, 1H), 7.06 (s, 1H), 3.67 (q, J=6.6 Hz, 1H), 2.49 (s, 3H), 1.46 (d, J=6.6 Hz, 3H). ¹⁹F NMR (376 MHz, DMSO) δ −130.63 (d, J=9.9 Hz). The racemate was separated by chiral chromatography (SFC Lux cellulose 2 from Phenomenex, CO₂+isopropanol 20%). Chiral purity 100%; rt=2.46 min. (second eluting enantiomer). For information, first eluting enantiomer rt=2.03 min. Both measured by HPLC, LuxCell2, Solvent: Heptane 50%−ethanol 50%−DEA 0.1%.

### Intermediate L5: enantiomer (10R) or (10S) of 3-chloro-5,10-dimethyl-4,8,12-triazatricyclo[9.4.0.0^{2,7}]pentadeca-1(11),2,4,6,12,14-hexaen-9-one

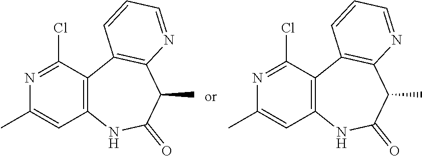

### Step 1: Synthesis of ethyl 2-(3-bromo-2-pyridyl)acetate L5_1

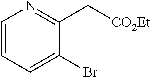 [SMILES: CCOC(=O)Cc1ncccc1Br]

At −78° C., to a solution of 3-bromo-2-methyl-pyridine (5.00 g, 29.1 mmol) in dry THE (100 mL) was added LiHMDS (1 M solution in THF, 58 mL, 58.0 mmol) and the reaction mixture was stirred at 0° C. for 1 h. Diethyl carbonate (5.15 g, 43.6 mmol) was added at 0° C. and the reaction mixture was stirred at room temperature for 15 min. The reaction mixture was treated with H₂O (200 mL) and extracted with EtOAc (2×200 mL). The combined organic layer was dried over anhydrous Na₂SO₄ and concentrated under vacuum. Purification by column chromatography on silica gel (using DCM as eluent) afforded the title compound (5.00 g, yield: 71%) as a brown oil. LC-MS (Method B6): [M+H]⁺ m/z: 243.8, rt: 1.71 min, purity: 94%. ¹H NMR (400 MHz, DMSO-d₆) δ 1.18 (t, J=6.8 Hz, 3H), 3.98 (s, 2H), 4.11 (q, J=6.8 Hz, 2H), 7.27-7.30 (m, 1H), 8.08 (d, J=8.4 Hz, 1H), 8.50 (d, J=4.4 Hz, 1H).

### Step 2: Synthesis of ethyl 2-(3-bromo-2-pyridyl)propanoate L5_2

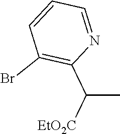

Ethyl 2-(3-bromo-2-pyridyl)acetate (Intermediate L5_1, 30.0 g, 117 mmol) was dissolved in dry THE (400 mL) and cooled to 0° C. A 1 M solution of lithium bis(trimethylsilyl)amide in THE (134 mL, 134 mmol) was added dropwise and stirred for 30 min. Iodomethane (21.5 g, 152 mmol) was added and the reaction mixture was stirred for a further 1 h at 0° C. The solution was concentrated under vacuum and the residue was purified by flash chromatography on silica gel (using a gradient of 0 to 60% MTBE in iso-hexane as eluent) to afford the title compound (29.5 g, yield: 96%) as a light-yellow oil. LC-MS (Method A7) m/z: [M+H]⁺: 258/260; rt: 1.93 min; purity: 99%. ¹H NMR (400 MHz, CDCl₃) δ 8.51 (dd, J=4.6, 1.5 Hz, 1H), 7.85 (dd, J=8.0, 1.5 Hz, 1H), 7.06 (dd, J=8.0, 4.6 Hz, 1H), 4.36 (q, J=7.1 Hz, 1H), 4.17 (q, J=7.1 Hz, 2H), 1.55 (d, J=7.1 Hz, 3H), 1.20 (t, J=7.1 Hz, 3H).

### Step 3: Synthesis of ethyl 2-[3-(4,4,5,5-tetramethyl-1,3,2-dioxaborolan-2-yl)-2-pyridyl]propanoate L5_3

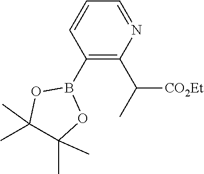 [SMILES: CCOC(=O)C(C)c1ncccc1B1OC(C)(C)C(C)(C)O1]

Ethyl 2-(3-bromo-2-pyridyl)propanoate (Intermediate L5_2, 30.0 g, 116 mmol), bis(pinacolato)diboron (35.4 g, 139 mmol) and potassium acetate (45.6 g, 465 mmol) were suspended in dry 1,4-dioxane (500 mL) and the reaction mixture was degassed with nitrogen for 10 min. Pd(dppf)C2l (5.95 g, 8.14 mmol) was added and the reaction mixture was stirred at 80° C. for 24 h. The reaction mixture was cooled to room temperature and filtered through a small pad of Celite®. The filter cake was rinsed with EtOAc (200 mL) and the filtrate was concentrated under vacuum. The residue was purified by flash chromatography on silica gel (using a gradient of 0 to 100% MTBE in iso-hexane as eluent) to afford the title compound (32.3 g, yield: 45%) as a yellow oil. ¹H NMR (400 MHz, CDCl₃) δ 8.60 (dd, J=4.8, 2.0 Hz, 1H), 8.10 (d, J=7.4 Hz, 1H), 7.17 (d, J=6.6 Hz, 1H), 4.71 (d, J=7.1 Hz, 1H), 4.14 (q, J=7.2 Hz, 2H), 1.55-1.51 (m, 3H), 1.34 (s, 12H), 1.18 (d, J=6.9 Hz, 3H).

### Step 4: Synthesis of enantiomer (10R) or (10S) of 3-chloro-5,10-dimethyl-4,8,12-triazatricyclo[9.4.0.0²⁷]pentadeca-1(11),2,4,6,12,14-hexaen-9-one L5

A mixture of 3-bromo-2-chloro-6-methyl-pyridin-4-amine (Intermediate L4_1, 400 mg, 1.81 mmol), ethyl 2-[3-(4,4,5,5-tetramethyl-1,3,2-dioxaborolan-2-yl)-2-pyridyl]propanoate (Intermediate L5_3, 1.10 g, 2.53 mmol), cesium fluoride (768 mg, 5.06 mmol) and bis(di-tert-butyl(4-dimethylaminophenyl)phosphine)dichloropalladium(II) (64 mg, 0.090 mmol) in 1,4-dioxane (34 mL) and water (1.7 mL), was purged with nitrogen then heated at reflux for 18 h. The reaction was allowed to cool to room temperature, then filtered through a bed of Celite*and washed with EtOAc (100 mL). The filtrate was concentrated under vacuum then taken up in absolute EtOH (16 mL) to which potassium carbonate (499 mg, 3.61 mmol) was added and the reaction mixture was heated at 65° C. for 4 h. The reaction mixture was concentrated under vacuum, then water (75 mL) was added and the reaction mixture was extracted with EtOAc (3×75 mL). The combined organic layers were dried over MgSO₄, filtered and concentrated under vacuum. The residue was purified by flash chromatography on silica gel (using a gradient of 0 to 100% EtOAc in iso-hexane as eluent) to afford the title compound (178 mg, yield: 35%) as an off-white solid. LC-MS (Method A7) m/z: [M+H]⁺: 274; rt: 1.45 min; purity: 99%. ¹H NMR (400 MHz, DMSO-d₆) δ 10.69 (s, 1H), 8.64 (dd, J=4.7, 1.7 Hz, 1H), 8.18 (dd, J=7.9, 1.7 Hz, 1H), 7.44 (dd, J=7.9, 4.7 Hz, 1H), 7.05 (s, 1H), 3.64 (q, J=6.6 Hz, 1H), 2.48 (s, 3H), 1.47 (d, J=6.6 Hz, 3H).

The racemate was separated by chiral chromatography (SFC Chiralpak AS from Daicel, CO₂+isopropanol 20%). Chiral purity 98.6%; rt=1.79 min. (second eluting enantiomer, L5). For information, first eluting enantiomer: rt=1.62 min. Both measured by HPLC, Chiralpak AS from Daicel, Solvent: Heptane 50%−Ethanol 50%−DEA 0.1%.

### Intermediate L6: N-(4-acetylphenyl)-2-(3,5-difluoro-10-methyl-9-oxo-4,8,12-triazatricyclo[9.4.0.0^{2,7}]pentadeca-1(11),2,4,6,12,14-hexaen-8-yl)acetamide

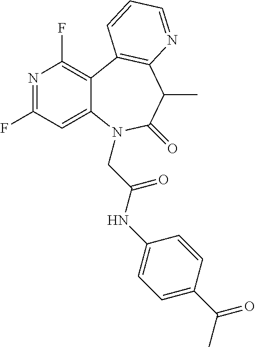 [SMILES: CC(=O)c1ccc(NC(=O)CN2C(=O)C(C)c3ncccc3-c3c2cc(F)nc3F)cc1]

### Step 1: Synthesis of 2,6-difluoro-3-(4,4,5,5-tetramethyl-1,3,2-dioxaborolan-2-yl)pyridin-4-amine L6_1

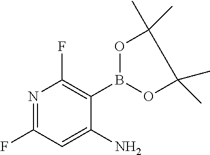 [SMILES: CC1(C)OB(c2c(N)cc(F)nc2F)OC1(C)C]

In a 60 mL vial, under N₂ atmosphere, a suspension of 4,4′-di-tert-butyl-2,2′-dipyridyl (63 mg, 0.23 mmol, 0.03 eq.) and (1,5-cyclooctadiene)(methoxy)iridium(I) dimer (76 mg, 0.115 mmol, 0.015 eq.) in dry THE (1.2 mL) was added to a solution of 2,6-difluoropyridin-4-amine (1.0 g, 7.740 mmol) and bis(pinacolato)diboron (3.9 g, 15 mmol, 2.0 eq.) in dry THE (5.8 mL). The reaction mixture was flushed with N₂ and stirred at 80° C. for 20 h. The reaction mixture was slowly poured into MeOH (50 mL) then concentrated to dryness. The brown residue was purified by column chromatography on silica gel (using a gradient of 0% to 30% EtOAc in DCM as eluent) to afford the title Intermediate as a white solid (1.73 g, yield: 71%). LC-MS (Method B1) m/z: [M+H]⁺: 257.3, rt: 1.15 min, purity: 81%. ¹H NMR (400 MHz, DMSO-d₆) δ 1.29 (s, 12H), 6.10 (s, 1H), 6.87 (bs, 2H).

### Step 2: Synthesis of 3,5-difluoro-10-methyl-4,8,12-triazatricyclo[9.4.0.0^{2,7}]pentadeca-1(11),2(7),3,5,12,14-hexaen-9-one L6_2

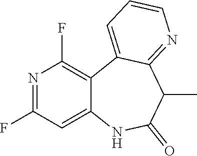 [SMILES: CC1C(=O)Nc2cc(F)nc(F)c2-c2cccnc21]

To 2,6-difluoro-3-(4,4,5,5-tetramethyl-1,3,2-dioxaborolan-2-yl)pyridin-4-amine (Intermediate L6_1, 48.2 g, 0.19 mmol) was added an aq. solution of potassium phosphate (2M, 190 mL), followed by a solution of ethyl 2-(3-bromo-2-pyridyl)propanoate (Intermediate L5_2, 44.0 g, 170 mmol) in dioxane (380 mL). Under nitrogen atmosphere, 2-dicyclohexylphosphino-2,6-dimethoxybiphenyl (8.1 g, 19.0 mmol) and tris(dibenzylideneacetone)dipalladium(0) (8.7 g, 9.4 mmol) were added and the resulting mixture was heated at 80° C. (internal temperature) and stirred for 1.5 hours. The reaction mixture was cooled to 20° C. (internal temperature), filtered through a pad of celite, washed with ethyl acetate (2×500 mL) and the filtrate was concentrated under vacuum. The resulting mixture was diluted with ethyl acetate (800 mL) and washed with water (2×500 mL), saturated sodium bicarbonate (500 mL), brine (500 mL), dried over sodium sulfate and concentrated to dryness to give the crude product (95.5 g) as a dark brown residue. Purification by column chromatography on silica gel (using a gradient of 0% to 100% ethyl acetate in iso-hexane as eluent) afforded ethyl 2-[3-(4-amino-2,6-difluoro-3-pyridyl)-2-pyridyl]propanoate (34.6 g, 90 mmol, yield: 48%) as a tan oil. LC-MS (Method B9) m/z: [M+H]⁺: =308.2, purity >99%, rt: 1.30 and 1.41 min as a mixture of diastereoisomers.

Under nitrogen atmosphere, to a solution of ethyl 2-[3-(4-amino-2,6-difluoro-3-pyridyl)-2-pyridyl]propanoate (42.8 g, 110 mmol) in anhydrous THE (450 mL) at 0° C., was added a 1M solution of lithium bis(trimethylsilyl)amide in THE (130 mL, 130 mmol) via a dropping funnel. The resulting mixture was stirred at 0° C. for 1 hour and then allowed to warm to room temperature and stirred for 1 hour. The reaction mixture was neutralized with saturated ammonium chloride solution (500 mL) and water (250 mL). Ethyl acetate (500 mL) was then added and stirred at room temperature for 15 minutes. The separated aqueous layer was extracted with ethyl acetate (500 mL) and the combined organic extracts were washed with water (2×500 mL), brine (500 mL), dried over sodium sulfate, and evaporated under vacuum. The crude solid was slurried in ethyl acetate (100 mL), iso-hexane (100 mL) was added and the solid was collected by filtration, washed with iso-hexane (50 mL×2) and dried under vacuum to afford the title intermediate L6_2 (22.33 g, yield: 77%) as an off-white solid. LC-MS (Method B9) m/z: [M+H]⁺: 262.0, rt: 1.13 min, purity >99%. ¹H NMR (300 MHz, DMSO-d₆) δ 11.04 (s, 1H), 8.68 (dd, J=4.7, 1.7 Hz, 1H), 8.11 (ddd, J=7.9, 4.6, 1.7 Hz, 1H), 7.47 (ddd, J=7.9, 4.8, 0.5 Hz, 1H), 6.87 (d, J=1.1 Hz, 1H), 3.72 (q, J=6.6 Hz, 1H), 1.49 (d, J=6.6 Hz, 3H).

### Step 3: Synthesis of N-(4-acetylphenyl)-2-(3,5-difluoro-10-methyl-9-oxo-4,8,12-triazatricyclo[9.4.0.0^{2,7}]pentadeca-1(11),2,4,6,12,14-hexaen-8-yl)acetamide L6

To a solution of 3,5-difluoro-10-methyl-4,8,12-triazatricyclo[9.4.0.0^{2,7}]pentadeca-1(11),2(7),3,5,12,14-hexaen-9-one L6_2 (646 mg, 2.5 mmol) and N-(4-acetylphenyl)-2-chloro-acetamide (Intermediate C1, 520 mg, 2.5 mmol) in DMF (15 mL) were added K₂CO₃ (1030 mg, 7.4 mmol) and KI (422 mg, 2.5 mmol). The reaction mixture was then stirred at room temperature overnight. The reaction mixture was poured in 50 mL of AcOEt. The organic layer was washed with 100 mL of water, 100 mL of NaCl solution and with 100 mL of water. The organic layer was dried over MgSO₄, filtered and the solvent was removed under high vacuum to give the crude product (yield 94%). LC-MS (Method A1_S) m/z: [M+H]⁺: 437.1, rt: 1.25 min, purity 92%. The crude product is used as such in the next step.

### Intermediate L7: 3-chloro-5-(2,2-difluoro-5-azaspiro[2.3]hexan-5-yl)-4,8,12-triazatricyclo[9.4.0.0^{2,7}]pentadeca-1(11),2,4,6,12,14-hexaen-9-one

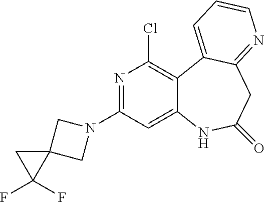 [SMILES: O=C1Cc2ncccc2-c2c(cc(N3CC4(C3)CC4(F)F)nc2Cl)N1]

### Step 1: Synthesis of tert-butyl N-[2-chloro-6-(2,2-difluoro-5-azaspiro[2.3]hexan-5-yl)-4-pyridyl]carbamate L7_1

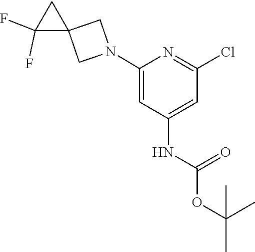 [SMILES: CC(C)(C)OC(=O)Nc1cc(Cl)nc(N2CC3(C2)CC3(F)F)c1]

Under N₂ atmosphere, to a solution of Tert-butyl (2,6-dichloropyridin-4-yl)carbamate (300 mg, 1.08 mmol) and 1,1-Difluoro-5-azaspiro[2.3]hexane hydrochloride (177 mg, 1.08 mmol) in dry Toluene (3.2 mL) at room temperature were added Pd₂dba₃ (51 mg, 0.05 mmol), BINAP (70 mg, 0.11 mmol) and potassium tert-butoxide (273 mg, 2.38 mmol). The reaction mixture was stirred at 90° C. for 16 h. After completion, the reaction mixture was filtered over celite, rinsed with EtOAc and concentrated under vacuum. The residue was purified by flash chromatography on silica gel (using a gradient of 0 to 100% EtOAc in Heptane as eluent) to afford the title compound as a white solid (247 mg, yield: 66%). LC-MS (Method A1_S) m/z: [M+H]⁺: 346.1; rt: 1.59 min; purity: 99%. ¹H NMR (400 MHz, DMSO-d₆) δ 9.80 (s, 1H), 6.77 (d, J=1.5 Hz, 1H), 6.53 (d, J=1.5 Hz, 1H), 4.05 (s, 4H), 1.74 (t, J=8.9 Hz, 2H), 1.47 (s, 9H). ¹⁹F NMR (376 MHz, DMSO-d₆) δ −137.58 (t, J=8.9 Hz).

### Step 2: Synthesis of 2-chloro-6-(2,2-difluoro-5-azaspiro[2.3]hexan-5-yl)pyridin-4-amine, trifluoroacetic acid salt L7_2

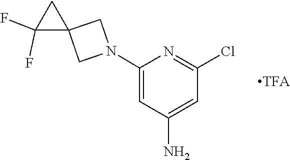 [SMILES: Nc1cc(Cl)nc(N2CC3(C2)CC3(F)F)c1]

To a solution of Intermediate L7_1 (247 mg, 0.71 mmol) in DCM (2.9 mL) at room temperature was added trifluoroacetic acid (2.7 mL, 35.7 mmol). The reaction mixture was stirred at room temperature for 2 h before being concentrated under vacuum. The residue was used as TFA salt in the next step without further purification. LC-MS (Method A1_S) m/z: [M+H]⁺: 246.0; rt: 0.89 min; purity: 88%.

### Step 3: Synthesis of 2-chloro-6-(2,2-difluoro-5-azaspiro[2.3]hexan-5-yl)-3-iodo-pyridin-4-amine L7_3

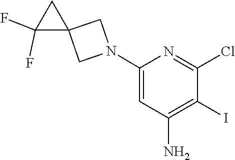 [SMILES: Nc1cc(N2CC3(C2)CC3(F)F)nc(Cl)c1I]

To a solution of Intermediate L7_2 (1.00 mmol) in MeCN (2 mL) was added a solution of N-iodosuccinimide (213 mg, 0.90 mmol) in MeCN (2 mL). The resulting mixture was stirred at room temperature for 2 h. Extra N-iodosuccinimide (70 mg, 0.30 mmol) was added and the reaction mixture was further stirred at room temperature for 1 h. After completion, the reaction mixture was concentrated under vacuum. The residue was purified by flash chromatography on silica gel (using a gradient of 0 to 25% EtOAc in Heptane as eluent) to afford the title compound as a brownish solid (240 mg, yield: 64%). LC-MS (Method A1_S) m/z: [M+H]⁺: 371.9; rt: 1.39 min; purity: 100%. ¹H NMR (400 MHz, DMSO-d₆) δ 6.17 (s, 2H), 5.61 (s, 1H), 3.99-3.91 (m, 4H), 1.73 (t, J=8.9 Hz, 2H). ¹⁹F NMR (376 MHz, DMSO-d₆) δ −137.57 (t, J=8.9 Hz).

### Step 4: Synthesis of ethyl 2-[3-(4,4,5,5-tetramethyl-1,3,2-dioxaborolan-2-yl)-2-pyridyl]acetate L7_4

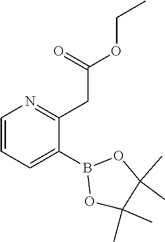 [SMILES: CCOC(=O)Cc1ncccc1B1OC(C)(C)C(C)(C)O1]

Under inert atmosphere, to a suspension of ethyl 2-(3-bromo-2-pyridyl)acetate (Intermediate L5_1, 5.00 g, 20.5 mmol), bis(pinacolato)diboron (CAS 73183-34-3, 6.24 g, 24.6 mmol), potassium acetate (8.0 g, 81.9 mmol), 3 Å molecular sieves (3 g) in 1,4-dioxane (200 mL), was added [1,1′-bis(diphenylphosphino)ferrocene]dichloropalladium(II) (749 mg, 1.02 mmol). The suspension was heated at reflux overnight then cooled to room temperature. It was filtered on a pad of Celite® and washed with ethyl acetate (400 mL). The filtrate was concentrated under vacuum and the residue was purified by flash chromatography on silica gel (using 0 to 100% tertbutylmethylether in cyclohexane as eluent) to give the title compound as a brown oil. (2.35 g, yield: 37%). LC-MS (Method B5) m/z: [M+H]⁺: 292.2; rt: 2.08 min, purity: 95%. ¹H NMR (400 MHz, CDCl₃) δ 8.60 (dd, J=4.9, 1.9 Hz, 1H), 8.14 (dd, J=7.6, 1.9 Hz, 1H), 7.23 (dd, J=7.6, 4.9 Hz, 1H), 4.22 (s, 2H), 4.18 (q, J=7.1 Hz, 2H), 1.35 (s, 12H), 1.28-1.24 (m, 3H).

### Step 5: Synthesis of 3-chloro-5-(2,2-difluoro-5-azaspiro[2.3]hexan-5-yl)-4,8,12-triazatricyclo[9.4.0.0^{2,7}]pentadeca-1(11),2,4,6,12,14-hexaen-9-one L7

The reaction was split in 3 batches. Under N₂ atmosphere, to a solution of Intermediate L7_3 (60 mg, 0.16 mmol) and ethyl 2-(3-(4,4,5,5-tetramethyl-1,3,2-dioxaborolan-2-yl)pyridin-2-yl)acetate L7_4 (73 mg, 0.24 mmol) in dry Toluene (0.81 mL) were added K₃PO₄ (105 mg, 0.48 mmol), CataCXium A (6 mg, 0.02 mmol) followed by Pd₂dba₃ (15 mg, 0.02 mmol). The resulting mixture was stirred at 100° C. for 16 h. After completion, the reaction mixture was filtered over celite, rinsed with EtOAc and concentrated under vacuum. The residue was dissolved in dry THE (0.8 mL) before addition of LiHMDS (1 M in THF, 480 μL, 0.48 mmol). The resulting mixture was stirred at room temperature for 1 h. After complete conversion, the reaction mixture was quenched with water and extracted with EtOAc (3×). The combined organic layers were dried over MgSO₄, filtered and concentrated under vacuum. The residue was purified by flash chromatography on silica gel (using a gradient of 0 to 100% EtOAc in Heptane as eluent) to afford the title compound as a brown solid (16 mg, yield: 23%). LC-MS (Method A1_S) m/z: [M+H]⁺: 363.0; rt: 1.13 min; purity: 85%.

### Intermediate L8: 2-(1-fluoro-3-methyl-6-oxo-7H-pyrido[4,3-d][3]benzazepin-5-yl)acetic acid, hydrochloride salt

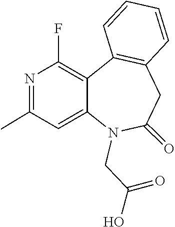 [SMILES: Cc1cc2c(c(F)n1)-c1ccccc1CC(=O)N2CC(=O)O]

### Step 1: Synthesis of 1-fluoro-3-methyl-5,7-dihydropyrido[4,3-d][3]benzazepin-6-one L8_1

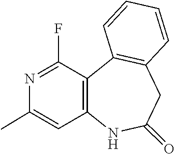 [SMILES: Cc1cc2c(c(F)n1)-c1ccccc1CC(=O)N2]

Under argon atmosphere, a suspension of 2-fluoro-6-methyl-3-(4,4,5,5-tetramethyl-1,3,2-dioxaborolan-2-yl)pyridin-4-amine (Intermediate L1_4, 3.00 g, 11.9 mmol), Methyl 2-(2-bromophenyl)acetate (1.92 mL, 11.9 mmol), K₂CO₃ (4.98 g, 35.7 mmol) and Pd(dppf)Cl₂ (436 mg, 0.60 mmol) in 1,4-Dioxane (40 mL) and water (2.4 mL) was stirred at 100° C. for 18 h. After completion, the reaction mixture was quenched with water and extracted with EtOAc. The organic layer was washed with water (2 x), with brine, dried over MgSO₄, filtered and concentrated under vacuum. The residue was triturated in DCM, filtered, rinsed with DCM and vacuum-dried to afford the title compound as a beige solid (944 mg, yield: 33%). LC-MS (Method B1_S) m/z: [M+H]⁺: 243.1; rt: 1.10 min; purity: 100%. ¹H NMR (400 MHz, DMSO-d₆) δ 10.60 (s, 1H), 7.64 (t, J=6.3 Hz, 1H), 7.50-7.37 (m, 3H), 6.96 (s, 1H), 3.49 (s, 2H), 2.43 (s, 3H).

### Step 2: Synthesis of tert-butyl 2-(1-fluoro-3-methyl-6-oxo-7H-pyrido[4,3-d][3]benzazepin-5-yl)acetate L8_2

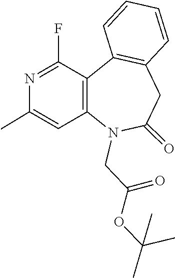 [SMILES: Cc1cc2c(c(F)n1)-c1ccccc1CC(=O)N2CC(=O)OC(C)(C)C]

To a suspension of Intermediate L8_1 (892 mg, 3.68 mmol), K₂CO₃ (1.03 g, 7.36 mmol), potassium iodide (62 mg, 0.37 mmol) in DMF (15 mL) was added. Tert-butyl bromoacetate (666 μL, 4.42 mmol) at room temperature. The resulting mixture was stirred at room temperature for 18 h. After completion, water was added. The resulting precipitate was collected by filtration, rinsed with water, Et₂O and vacuum-dried to afford the title compound as a white solid (1.29 g, yield: 95%). LC-MS (Method B1_S) m/z: [M+H]⁺: 357.2; rt: 1.48 min; purity: 99%.

### Step 3: Synthesis of 2-(1-fluoro-3-methyl-6-oxo-7H-pyrido[4,3-d][3]benzazepin-5-yl)acetic acid, hydrochloride salt L8

To a solution of Intermediate L8_2 (1.24 g, 3.47 mmol) in DCM (17 mL) was added HCl (4.0 M in 1,4-Dioxane, 8.67 mL, 34.7 mmol) at room temperature. The resulting mixture was stirred at room temperature for 18 h. After completion, the precipitate was collected by filtration, rinsed with DCM (3×) and vacuum-dried to afford the title compound as HCl salt (1.07 g, yield: 86%). LC-MS (Method B1_S) m/z: [M+H]⁺: 301.0; rt: 0.77 min; purity: 100%. ¹H NMR (400 MHz, DMSO-d₆) δ 7.66 (dd, J=7.2, 5.4 Hz, 1H), 7.49-7.39 (m, 3H), 7.23 (s, 1H), 4.46-4.31 (m, 2H), 3.64-3.49 (m, 2H), 2.48 (s, 3H). Proton for COOH not observed.

### Intermediate L9: enantiomer (10R) or (10S) of 2-[3-fluoro-5-methoxy-10-methyl-9-oxo-4,8,12-triazatricyclo[9.4.0.0^{2,7}]pentadeca-1(11),2(7),3,5,12,14-hexaen-8-yl]acetic acid, hydrochloride salt

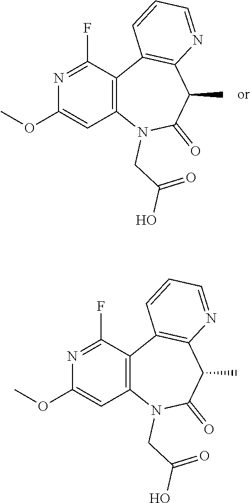

### Step 1: Synthesis of 2-fluoro-6-methoxy-pyridin-4-amine L9_1

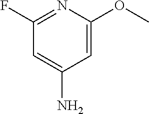

A solution of 2,6-difluoropyridin-4-amine (760 mg, 5.84 mmol) and sodium methoxide (5.4 M solution in methanol, 2.4 mL, 12.9 mmol) in dry THE (29 mL) was heated at reflux for 4 h. The reaction mixture was diluted with water (30 mL) then extracted with EtOAc (3×30 mL). The combined extracts were washed with brine (60 mL), dried over Na₂SO₄, filtered and concentrated under vacuum. The residue was purified by flash chromatography on silica gel (using a gradient of 0 to 60% EtOAc in iso-hexane as eluent) to give the title compound (725 mg, yield: 78%) as a yellow oil. LC-MS (Method A7) m/z: [M+H]⁺: 143; rt: 0.96 min; purity: 55%. ¹H NMR (400 MHz, CDCl₃) δ 5.82 (dd, J=1.6, 1.0 Hz, 1H), 5.78 (d, J=1.6 Hz, 1H), 4.25 (s, 2H), 3.87 (s, 3H).

### Step 2: Synthesis of 2-fluoro-6-methoxy-3-(4,4,5,5-tetramethyl-1,3,2-dioxaborolan-2-yl)pyridin-4-amine L9_2

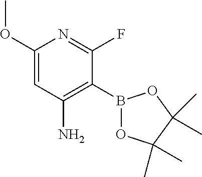 [SMILES: COc1cc(N)c(B2OC(C)(C)C(C)(C)O2)c(F)n1]

A solution of 2-fluoro-6-methoxy-pyridin-4-amine (Intermediate L9_1, 211 mg, 1.34 mmol) in dry THE (6.4 mL) was degassed with nitrogen for 5 min then 4,4,5,5-tetramethyl-1,3,2-dioxaborolane (0.20 mL, 1.43 mmol) was added dropwise. The resulting mixture was stirred at room temperature for 30 min then bis(pinacolato)diboron (CAS 73183-34-3, 376 mg, 1.48 mmol), 4-tert-butyl-2-(4-tert-butyl-2-pyridyl)pyridine (36 mg, 0.134 mmol) and (1,5-cyclooctadiene)(methoxy)iridium(I) dimer (45 mg, 0.07 mmol) were added. The resulting mixture was degassed with nitrogen for 5 min then stirred at 80° C. overnight. The reaction mixture was filtered through a pad of Celite, washed with EtOAc (10 mL) and the filtrate was concentrated under vacuum. The residue was purified by flash chromatography on silica gel (using a gradient of 0 to 100% EtOAc in iso-hexane as eluent) to give the title compound (265 mg, yield: 59%) as a white solid. LC-MS (Method A7) m/z: [M+H]⁺: 269; rt: 2.02 min; purity: 70%. ¹H NMR (400 MHz, DMSO-d₆) δ 6.57 (s, 2H), 6.29 (t, J=1.3 Hz, 1H), 2.16 (s, 3H), 1.28 (s, 12H).

### Step 3: Synthesis of ethyl 2-[3-(4-amino-2-fluoro-6-methoxy-3-pyridyl)-2-pyridyl]propanoate L9_3

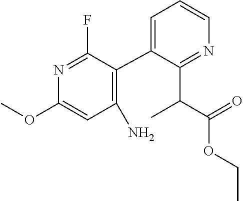 [SMILES: CCOC(=O)C(C)c1ncccc1-c1c(N)cc(OC)nc1F]

A suspension of ethyl 2-(3-bromo-2-pyridyl)propanoate (Intermediate L5_2, 2.90 g, 11.0 mmol), 2-fluoro-6-methoxy-3-(4,4,5,5-tetramethyl-1,3,2-dioxaborolan-2-yl)pyridin-4-amine (Intermediate L9_2, 4.56 g, 13.8 mmol) and CsF (5.85 g, 38.5 mmol) were poured in a mixture of toluene (12.0 mL), EtOH (6.0 mL) and water (6.0 mL). The mixture was purged with nitrogen for 10 min and PEPPSI™-IPr (CAS 905459-27-0, 748 mg, 1.10 mmol) was then added. The reaction mixture was purged with nitrogen for an additional 5 min and was heated at 80° C. for 16 h. The reaction mixture was cooled to room temperature, filtered through a pad of Celite* and washed with EtOAc (50 mL). The filtrate was concentrated under vacuum and the residue was purified by flash chromatography on silica gel (using a gradient of 0 to 5% MeOH in DCM as eluent) to afford the title compound as a yellow solid (1.73 g, yield: 42%). LC-MS (Method A7) m/z [M+H]⁺: 320.2; rt: 1.71 min; purity: 87%.

### Step 4: Synthesis of enantiomer (10S) or (10R) of 3-fluoro-5-methoxy-10-methyl-4,8,12-triazatricyclo[9.4.0.0^{2,7}]pentadeca-1(11),2(7),3,5,12,14-hexaen-9-one L9_4

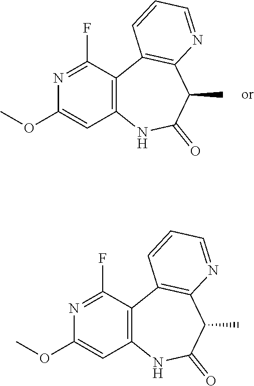

To a solution of ethyl 2-[3-(4-amino-2-fluoro-6-methoxy-3-pyridyl)-2-pyridyl]propanoate (Intermediate L9_3, 1.96 g, 5.52 mmol) in dry THE (50.0 mL) was added dropwise a 1M solution of lithium bis(trimethylsilyl)amide in THE (11.0 mL, 11.0 mmol) and the reaction mixture was stirred for 1 h at room temperature. The reaction mixture was treated with ice-water (10 mL) and extracted with EtOAc (3×20 mL) and the combined organic extracts were dried over magnesium sulfate, filtered and concentrated under vacuum to afford the title compound as a light brown powder (1.59 g, quantitative yield). LC-MS (Method A7) m/z [M+H]P: 274.1; rt: 1.59 min; purity: 96%. ¹H NMR (400 MHz, DMSO-d₆) δ 10.72 (s, 1H), 8.62 (dd, J=4.7, 1.7 Hz, 1H), 8.04 (ddd, J=7.9, 4.7, 1.7 Hz, 1H), 7.44 (dd, J=7.9, 4.7 Hz, 1H), 6.54 (s, 1H), 3.89 (s, 3H), 3.72-3.66 (m, 1H), 1.47 (d, J=6.6 Hz, 3H). The racemate was separated by chiral chromatography (SFC, Chiralpak IG from Daicel, CO₂+Methanol 20%). Chiral purity 100%. Rt=1.78 min (first eluting enantiomer). (For information, second eluting enantiomer: rt=2.24 min. Both measured by HPLC, Chiralpak IG from Daicel, Solvent: ACN 100%−DEA 0.1%).

### Step 5: Synthesis of enantiomer (10R) or (10S) of tert-butyl 2-(3-fluoro-5-methoxy-10-methyl-9-oxo-4,8,12-triazatricyclo[9.4.0.0^{2,7}]pentadeca-1(11),2(7),3,5,12,14-hexaen-8-yl)acetate L9_5

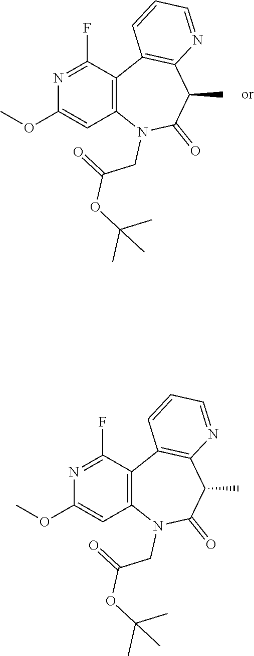

To a solution of Enantiomer (10S) or (10R) of 3-fluoro-5-methoxy-10-methyl-4,8,12-triazatricyclo[9.4.0.0^{2,7}]pentadeca-1(11),2(7),3,5,12,14-hexaen-9-one (Intermediate L9_4, 1.00 g, 3.66 mmol), KI (305 mg, 1.82 mmol) and K₂CO₃ (1.02 g, 7.31 mmol) in DMF (18 mL) was added tert-butyl bromoacetate (660 μL, 4.38 mmol). The resulting mixture was stirred at room temperature for 2 h. After complete conversion, water (80 mL) was added and the reaction mixture was extracted with EtOAc (3×80 mL). The combined organic extracts were washed with brine (2×200 mL), dried over MgSO₄, filtered, and concentrated under vacuum. The residue was purified by flash chromatography on silica gel (using a gradient of 0 to 40% EtOAc in Heptane as eluent) to afford the title compound (1.37 g, yield: 94%) as a white solid. LC-MS (Method A2) m/z: [M+H]⁺: 388.2; rt: 4.68 min; purity: 98%. LC-MS (Method B2) m/z: [M+H]⁺: 388.1; rt: 4.59 min; purity: 97%. ¹H NMR (400 MHz, DMSO-d₆) δ 8.63 (dd, J=4.8, 1.7 Hz, 1H), 8.08 (ddd, J=7.9, 4.5, 1.7 Hz, 1H), 7.47 (dd, J=7.9, 4.8 Hz, 1H), 6.80 (s, 1H), 4.52-4.39 (m, 2H), 3.93 (s, 3H), 3.81 (q, J=6.6 Hz, 1H), 1.48 (d, J=6.6 Hz, 3H), 1.25 (s, 9H). ¹⁹F NMR (376 MHz, DMSO-d₆) δ −71.73 (d, J=4.5 Hz). Chiral purity: 100%; rt=4.22 min (second eluting enantiomer). For information, first eluting enantiomer rt=3.22 min. Both measured by HPLC (Chiralpak IG-u from Daicel, EtOH 50%−heptane 50%−DEA 0.1%).

### Step 6: Synthesis of enantiomer (10R) or (10S) of 2-[3-fluoro-5-methoxy-10-methyl-9-oxo-4,8,12-triazatricyclo[9.4.0.0^{2,7}]pentadeca-1(11),2(7),3,5,12,14-hexaen-8-yl]acetic acid, hydrochloride salt L9

To a solution of enantiomer (10S) or (10R) of tert-butyl 2-(3-fluoro-5-methoxy-10-methyl-9-oxo-4,8,12-triazatricyclo[9.4.0.0^{2,7}]pentadeca-1(11).2(7).3,5,12,14-hexaen-8-yl)acetate (Intermediate L9_5, 1.36 g, 3.42 mmol) in DCM (34 mL) was added HCl (4 M in 1,4-dioxane, 8.55 mL, 34.2 mmol). The resulting mixture was stirred at room temperature for 5 h. Extra HCl (4 M in 1,4-Dioxane, 1.71 mL, 6.84 mmol) was added and the reaction mixture was further stirred at room temperature for 17 h. The reaction mixture was concentrated under vacuum to afford the title compound (HCl salt, 1.41 g, quantitative yield) as a white solid. LC-MS (Method A2) m/z: [M+H]⁺: 332.1; rt: 3.43 min; purity: 89%. LC-MS (Method B2) m/z: [M+H]⁺: 332.0; rt: 2.45 min; purity: 94%. ¹H NMR (400 MHz, DMSO-d₆) δ 8.63 (dd, J=4.8, 1.7 Hz, 1H), 8.08 (ddd, J=7.9, 4.5, 1.7 Hz, 1H), 7.48 (dd, J=7.9, 4.8 Hz, 1H), 6.82 (s, 1H), 4.45 (s, 2H), 3.92 (s, 3H), 3.84 (q, J=6.6 Hz, 1H), 1.48 (d, J=6.6 Hz, 3H). Proton for COOH not observed. ¹⁹F NMR (376 MHz, DMSO-d₆) δ −71.90 (d, J=4.5 Hz). Chiral purity: 100%; rt=2.74 min (first eluting enantiomer). For information, second eluting enantiomer rt=3.72 min. Both measured by HPLC (Lux Cellulose-2 from Phenomenex, EtOH 50%−heptane 50%−DEA 0.1%).

### Intermediate L10: 3-fluoro-14-methoxy-5,10-dimethyl-4,8,12-triazatricyclo[9.4.0.0^{2,7}]pentadeca-1(11),2(7),3,5,12,14-hexaen-9-one

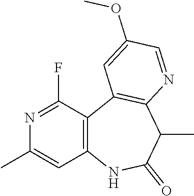

A mixture of Intermediate L1_4 (415 mg, 1.65 mmol), Intermediate L11_1 (395 mg, 1.37 mmol) and potassium phosphate tribasic (594 mg, 2.74 mmol) in toluene (11 mL) was degassed with nitrogen for 10 min before the addition of tris(dibenzylideneacetone)dipalladium(0) (128 mg, 0.14 mmol) and 2-dicyclohexylphosphino-2,6-dimethoxybiphenyl (57 mg, 0.14 mmol). The reaction mixture was heated at 90° C. for 3 h. After completion, the reaction mixture was filtered over celite with EtOAc and the filtrate wax concentrated under vacuum to give a yellow oil.

The crude oil was dissolved in toluene (13.7 mL) and lithium bis(trimethylsilyl)amide (1.5 M in THF, 2.7 mL) was added at room temperature under N₂ atmosphere. The reaction mixture was stirred at room temperature. After 1 h, the reaction mixture was poured into water and extracted twice with EtOAc. The combined organic layers were dried over MgSO₄, filtered, and concentrated to dryness to give a beige solid. The residue was triturated with Et₂O/DCM (75/25), the solid was filtered to afford the title compound (203 mg, yield: 52%). LC-MS (Method B1_S) m/z: [M+H]⁺: 288; rt: 1.05 min; purity: 89%.

### Intermediate L11: 14-methoxy-5,10-dimethyl-4,8,12-triazatricyclo[9.4.0.0^{2,7}]pentadeca-1(11),2(7),3,5,12,14-hexaen-9-one

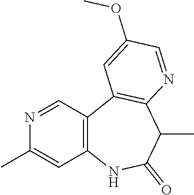

### Step 1: Synthesis of ethyl 2-(3-bromo-5-methoxy-2-pyridyl)propanoate L11_1

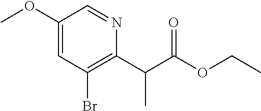 [SMILES: CCOC(=O)C(C)c1ncc(OC)cc1Br]

To a solution of ethyl 2-(3-bromo-5-fluoro-2-pyridyl)propanoate (L2_3, 1.0 g, 3.62 mmol) in dry DMF (18 mL) cooled at 0° C., was added a 25% solution of sodium methoxide in methanol (1.5 mL, 6.52 mmol) and the reaction mixture was allowed to reach room temperature over 1 hour. The reaction mixture was treated with water and extracted twice with EtOAc. The combined organic extracts were dried with brine, over MgSO₄, filtered off and concentrated to dryness to give the title intermediate L11_1 (848 mg, yield: 81%) as a crude yellow oil which was used in the next step without purification. LC-MS (method A2) m/z [M+H]⁺: 270.1; rt: 1.40 min. ¹H NMR (400 MHz, CDCl₃) δ 8.28 (d, J=2.6 Hz, 1H), 7.36 (d, J=2.6 Hz, 1H), 4.28 (q, J=7.2 Hz, 1H), 4.20 (q, J=7.2 Hz, 2H), 3.77 (s, 3H), 1.51 (d, J=7.2 Hz, 3H), 1.20 (t, J=7.2 Hz, 3H).

### Step 2: synthesis of 14-methoxy-5,10-dimethyl-4,8,12-triazatricyclo[9.4.0.0^{2,7}]pentadeca-1(11),2(7),3,5,12,14-hexaen-9-one L11

A suspension of 2-methyl-5-(4,4,5,5-tetramethyl-1,3,2-dioxaborolan-2-yl)pyridin-4-amine (CAS: 1668475-78-2, 1.04 g, 1.64 mmol), ethyl 2-(3-bromo-5-methoxy-2-pyridyl)propanoate (L11_1, 395 mg, 1.37 mmol) and potassium phosphate (594 mg, 2.74 mmol) in dry toluene (11 mL) was degassed with argon. Tris(dibenzylideneacetoneacetone)dipalladium(0) (128 mg, 0.18 mmol) and 2-dicyclohexylphosphino-2,6-dimethoxybenzyl (57 mg, 0.14 mmol) were added. The vial was sealed under argon atmosphere and the reaction mixture was heated at 90° C. for 3 h. After cooling to room temperature, the reaction mixture was filtered through a pad of celite and rinsed with EtOAc. The filtrate was concentrated under vacuum to give a crude oil which was dissolved again in dry toluene (14 mL). To this solution was added a 1.5 M solution of lithium bis(trimethylsilyl)amide in THE (2.7 mL) and the reaction mixture was stirred at room temperature for 1 hour. The reaction mixture was treated with water and extracted twice with EtOAc. The combined organic extracts were washed with brine, then dried over MgSO₄, filtered off and concentrated to dryness to give a brown solid. Trituration and sonication in a 75/25 mixture of Et₂O:DCM afforded the title product (258 mg, yield: 70%) as a white solid. LC-MS (method B2) m/z [M+H]P: 270.1; purity: 97%, rt: 0.89 min. ¹H NMR (400 MHz, DMSO-d₆) δ 8.62 (s, 1H), 8.31 (d, J=2.7 Hz, 1H), 7.59 (d, J=2.9 Hz, 1H), 6.81 (s, 1H), 3.89 (s, 3H), 3.59 (q, J=7.0 Hz, 1H), 2.44 (s, 4H), 1.43 (d, J=6.7 Hz, 3H).

### Intermediate L12: 3-methoxy-5,10-dimethyl-4,8,12-triazatricyclo[9.4.0.0^{2,7}]pentadeca-1(11),2,4,6,12,14-hexaen-9-one

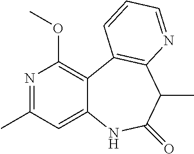

### Step 1: Synthesis of 3-bromo-2-methoxy-6-methyl-pyridin-4-amine L12_1

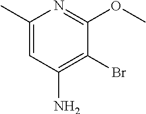

At 0° C., to a solution of 2-methoxy-6-methyl-pyridin-4-amine (505 mg, 3.47 mmol) in DCM (25 mL) was added a suspension of NBS (618 mg, 3.47 mmol) in DCM (10 mL) and the reaction mixture was stirred at 0° C. for 1 h. Water (40 mL) was added and the aqueous layer was extracted with DCM (2×30 mL). The combined organic extracts were washed with brine (80 mL), dried over Na₂SO₄, filtered and concentrated under vacuum. The residue was purified by flash chromatography on silica gel (using 0 to 4% MeOH (0.7 N in NH₃) in DCM as eluent) to give the title compound as a colorless oil (653 mg, yield: 86%). LC-MS (Method B5) m/z: [M+H]⁺: 217.1/219.1; rt: 1.57 min; purity >99%. ¹H NMR (400 MHz, CDCl₃) δ 6.16 (d, J=0.7 Hz, 1H), 4.47 (s, 2H), 3.97 (s, 3H), 2.31 (s, 3H).

### Step 2: Synthesis of ethyl 2-(4′-amino-2′-methoxy-6′-methyl-[3,3′-bipyridin]-2-yl)propanoate L12_2

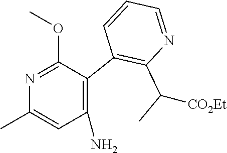 [SMILES: CCOC(=O)C(C)c1ncccc1-c1c(N)cc(C)nc1OC]

To a stirring mixture of 3-bromo-2-methoxy-6-methylpyridin-4-amine (Intermediate L12_1) (1.60 g, 7.37 mmol), ethyl 2-[3-(4,4,5,5-tetramethyl-1,3,2-dioxaborolan-2-yl)-2-pyridyl]propanoate (Intermediate L5_3, 3.29 g, 8.85 mmol) and a 1.5M solution of aq. K₂CO₃ (14.5 mL, 22.0 mmol) in dry 1,4-dioxane (100 mL) was added bis(di-tert-butyl(4-dimethylaminophenyl)phosphine)dichloropalladium(II) (522 mg, 0.737 mmol) and the reaction mixture was stirred at 90° C. for 2 h. The reaction mixture was cooled to room temperature and filtered through a small pad of celite. The filter cake was rinsed with EtOAc (150 mL) and the filtrate was concentrated under vacuum. The residue was purified by flash chromatography on silica gel (using a gradient of 0 to 100% (0.7 M NH₃/MeOH) in DCM as eluent) to afford the title compound (3.24 g, yield: 73%) as a brown oil. LC-MS (Method A7) m/z [M+H]⁺: 316.2; rt: 0.90 min; purity: 53%. ¹H NMR (400 MHz, CDCl₃) δ 8.61 (dd, J=4.8, 1.8 Hz, 1H), 7.50 (dd, J=7.7, 1.8 Hz, 1H), 7.25-7.22 (m, 1H), 6.21 (s, 1H), 4.17-4.02 (m, 2H), 3.86-3.75 (m, 6H), 2.38 (d, J=2.1 Hz, 3H), 1.42 (m, 3H), 1.16 (t, J=7.2 Hz, 3H).

### Step 3: Synthesis of 3-methoxy-5,10-dimethyl-4,8,12-triazatricyclo[9.4.0.0^{2,7}]pentadeca-1(11),2(7),3,5,12,14-hexaen-9-one L12_3

Ethyl 2-[3-(4-amino-2-methoxy-6-methyl-3-pyridyl)-2-pyridyl]propanoate (Intermediate L12_2, 3.24 g, 5.45 mmol) was dissolved in EtOH (70 mL) and potassium carbonate (1.50 g, 10.9 mmol) was added. The reaction mixture was stirred at 90° C. for 16 h. The reaction mixture was concentrated under vacuum and the residue was dissolved in water (30 mL) and extracted with EtOAc (4×50 mL). The combined organic extracts were dried over sodium sulfate, filtered and concentrated under vacuum to afford the title compound (1.99 g, yield: 92%) as a white solid. The product was taken to the next step without further purification. LC-MS (Method A7) m/z [M+H]⁺: 270.2; rt: 1.50 min; purity: 68%. ¹H NMR (400 MHz, DMSO-d₆) δ 10.45 (s, 1H), 8.56 (dd, J=4.7, 1.7 Hz, 1H), 8.11 (dd, J=8.0, 1.7 Hz, 1H), 7.37 (dd, J=7.9, 4.7 Hz, 1H), 6.74-6.64 (m, 1H), 3.89 (s, 3H), 3.46 (d, J=6.6 Hz, 1H), 2.41 (s, 3H), 1.46 (d, J=6.6 Hz, 3H).

### Intermediate L13: 3,14-difluoro-5,10-dimethyl-4,8,13-triazatricyclo[9.4.0.0^{2,7}]pentadeca-1(11),2(7),3,5,12,14-hexaen-9-one

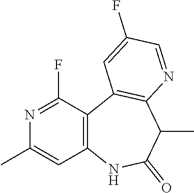

### Step 1: Synthesis of (E) and (Z) 4-bromo-2-fluoro-5-(2-methoxyvinyl)pyridine L13_1

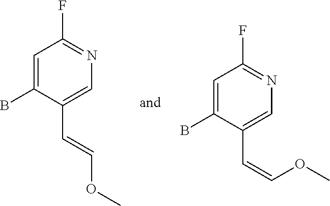

To a solution of (methoxymethyl)triphenylphosphonium chloride (184 mg, 0.54 mmol) in dry THE (2.40 mL) was added sodium bis(trimethylsilyl)amide (750 μL, 0.75 mmol, 1 M in THF) at 0° C., under N₂ atmosphere. The resulting mixture was stirred at 0° C. for 30 min before dropwise addition of a solution of 4-bromo-6-fluoronicotinaldehyde (100 mg, 0.47 mmol) in dry THE (2.40 mL). The resulting mixture was stirred at 0° C. for 30 min then at room temperature for 30 min. After complete conversion, the reaction mixture was quenched with water (30 mL) and extracted with EtOAc (3×30 mL). The combined organic layers were dried over MgSO₄, filtered and concentrated under vacuum. The residue was purified by flash chromatography on silica gel (using a gradient of 0 to 10% EtOAc in Heptane as eluent) to afford a mixture of isomers E and Z of the title compound as a colorless oil (66.9 mg, yield: 62%). LC-MS (Method A1_S) m/z: [M+H]⁺: 232.0/234.0; mixture of E/Z isomers, rt: 1.37 min & 1.40 min; purity: 100%. Isomer E: ¹H NMR (400 MHz, DMSO-d₆) δ 8.35 (s, 1H), 7.59 (d, J=2.9 Hz, 1H), 7.32 (d, J=12.8 Hz, 1H), 5.82 (d, J=12.8 Hz, 1H), 3.70 (s, 3H). 19F NMR (376 MHz, DMSO-d₆) δ −73.14 (d, J=2.9 Hz). Isomer Z: ¹H NMR (400 MHz, DMSO-d₆) δ 8.69 (s, 1H), 7.61 (d, J=2.9 Hz, 1H), 6.62 (d, J=7.0 Hz, 1H), 5.38 (d, J=7.0 Hz, 1H), 3.81 (s, 3H). ¹⁹F NMR (376 MHz, DMSO-d₆) δ −72.24 (d, J=2.9 Hz).

### Step 2: Synthesis of 2-(4-bromo-6-fluoro-3-pyridyl)acetaldehyde L13_2

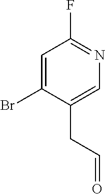 [SMILES: O=CCc1cnc(F)cc1Br]

A solution of hydrochloric acid (12 M in water, 300 μL, 3.49 mmol) diluted in water (300 μL) was added dropwise to a solution of Intermediate L13_1 (53 mg, 0.23 mmol) in acetone (5.7 mL). The resulting mixture was stirred at 50° C. for 3 h. After completion, the reaction mixture was concentrated under vacuum to afford the title compound as a brown solid directly used in the next step without purification (42.9 mg, yield: 86%). ¹H NMR (400 MHz, DMSO-d₆) δ 9.73 (s, 1H), 8.19 (s, 1H), 7.68 (d, J=2.7 Hz, 1H), 4.02 (s, 2H). ¹⁹F NMR (376 MHz, DMSO-d₆) δ −70.54.

### Step 3: Synthesis of 2-(4-bromo-6-fluoro-3-pyridyl)acetic acid L13_3

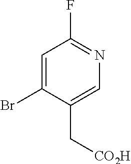 [SMILES: O=C(O)Cc1cnc(F)cc1Br]

To a solution of Intermediate L13_2 (42.9 mg, 0.20 mmol) and 2-methyl-2-butene (210 μL, 1.96 mmol) in tert-butyl alcohol (2 mL) was added a solution of sodium dihydrogen phosphate (118 mg, 0.98 mmol) in water (0.8 mL) followed by the portionwise addition of sodium chlorite (27 mg, 0.24 mmol). The resulting mixture was stirred at room temperature for 1 h. After completion, the reaction mixture was quenched with water (20 mL) and a 4 M aqueous HCl solution (5 mL), followed by extraction with DCM (3×30 mL). The combined organic layers were dried over MgSO₄, filtered and concentrated under vacuum to afford the crude title compound as a yellow oil which was directly used in the next step without purification. LC-MS (Method A1_S) m/z: [M+H]⁺: 233.9/235.9; rt: 0.91 min; purity: 70%.

### Step 4: Synthesis of 2-(4-bromo-6-fluoro-3-pyridyl)propanoic acid L13_4

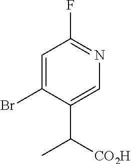

To a solution of crude Intermediate L13_3 (41.0 mg, 0.18 mmol) in dry THE (1.7 mL) at −78° C. under N₂ atmosphere was added dropwise lithium bis(trimethylsilyl)amide (1 M in THF, 350 μL, 0.35 mmol). The reaction mixture was stirred at −78° C. for 30 min. Iodomethane (12.1 μL, 0.19 mmol) was then added dropwise and the reaction mixture was stirred at room temperature for 2 h. After completion, the reaction mixture was quenched by addition of water (10 mL) followed by a 4 M aqueous HCl solution (10 mL). The mixture was extracted with DCM (2×20 mL) then with CHCl₃/iPrOH (4:1 v/v, 3×20 mL). The combined organic layers were dried over MgSO₄, filtered and concentrated under vacuum to afford the crude title compound as a brown oil which was directly used in the next step without purification. LC-MS (Method A1_S) m/z: [M+H]⁺: 247.9/249.9; rt: 1.04 min; purity: 60%.

### Step 5: Synthesis of methyl 2-(4-bromo-6-fluoro-3-pyridyl)propanoate L13_5

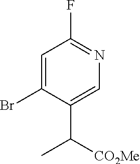

To a solution of crude Intermediate L13_4 (39.4 mg, 0.16 mmol) in dry acetonitrile (0.80 mL) was added potassium carbonate (66.5 mg, 0.47 mmol) followed by iodomethane (12.5 μL, 0.21 mmol). The reaction mixture was stirred at room temperature for 16 h. Extra iodomethane (12.5 μL, 0.21 mmol) was added and the reaction mixture was stirred at room temperature for 5 h. After completion, the reaction mixture was quenched with water (15 mL) and extracted with EtOAc (3×20 mL). The combined organic layers were dried over MgSO₄, filtered and concentrated under vacuum. The residue was purified by flash chromatography on silica gel (using a gradient of 0 to 20% EtOAc in Heptane as eluent) to afford the title compound as a yellow oil (12.5 mg, yield: 22% over 3 steps). LC-MS (Method A1_S) m/z: [M+H]⁺: 261.9/263.9; rt: 1.29 min; purity: 91%. ¹H NMR (500 MHz, CDCl₃) δ 8.14 (s, 1H), 7.20 (d, J=3.0 Hz, 1H), 4.12 (q, J=7.3 Hz, 1H), 3.71 (s, 3H), 1.57 (d, J=7.3 Hz, 3H). 19F NMR (471 MHz, CDCl₃) δ −69.81 (d, J=3.0 Hz).

### Step 6: Synthesis of 3,14-difluoro-5,10-dimethyl-4,8,13-triazatricyclo[9.4.0.0²⁷]pentadeca-1(11),2(7),3,5,12,14-hexaen-9-one L13

To a solution of Intermediate L13_5 (12.5 mg, 0.04 mmol) and Intermediate L1_4 (16.4 mg, 0.06 mmol) in dry toluene (0.22 mL) were added, under N₂ atmosphere, potassium phosphate tribasic (28.1 mg, 0.13 mmol), 2-dicyclohexylphosphino-2,6-dimethoxybiphenyl (2 mg, 0.005 mmol) followed by tris(dibenzylideneacetone)dipalladium(0) (4 mg, 0.004 mmol). The resulting mixture was stirred at 100° C. for 3 h. After completion, the reaction mixture was quenched with water (10 mL) and extracted with EtOAc (3×10 mL). The combined organic layers were dried over MgSO₄, filtered and concentrated under vacuum. The residue was dissolved in dry toluene (220 μL) before addition, under N₂ atmosphere, of lithium bis(trimethylsilyl)amide (1 M in THF, 130 μL, 0.13 mmol). The resulting mixture was stirred at room temperature for 2 h. After completion, the reaction mixture was concentrated under vacuum. The residue was purified by preparative HPLC (Purification Method P_B) to afford the title compound as a white solid (1.7 mg, yield: 14%). LC-MS (Method A4) m/z: [M+H]⁺: 276.1; rt: 3.37 min; purity: 100%.

### Intermediate L14: 3-methoxy-10-methyl-4,8,12-triazatricyclo[9.4.0.0^{2,7}]pentadeca-1(11),2(7),3,5,12,14-hexaen-9-one

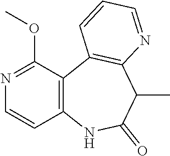 [SMILES: COc1nccc2c1-c1cccnc1C(C)C(=O)N2]

Step 1: Synthesis of 3-fluoro-10-methyl-4,8,12-triazatricyclo[9.4.0.0^{2,7}]pentadeca-1(11),2(7),3,5,12,14-hexaen-9-one L14_1

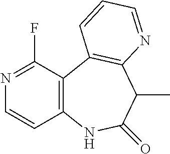 [SMILES: CC1C(=O)Nc2ccnc(F)c2-c2cccnc21]

To a solution of 4-amino-3-bromo-2-fluoropyridine (75 mg, 0.37 mmol) and ethyl 2-[3-(4,4,5,5-tetramethyl-1,3,2-dioxaborolan-2-yl)-2-pyridyl]propanoate (Intermediate L5_3, 244 mg, 0.74 mmol) in 1,4-dioxane (1.9 mL) were added K₂CO₃ (104 mg, 0.74 mmol) and water (82 μL). The reaction mixture was degassed with nitrogen for 5 min before addition of Pd[(Amphos)₂Cl]₂ (CAS 887919-35-9, 14 mg, 0.02 mmol) and the reaction mixture was stirred at 100° C. for 20 h. After cooling down to RT, water was added, and the reaction mixture was extracted with EtOAc (3×). The combined organic layers were washed with brine, dried over MgSO₄, filtered and concentrated to dryness. The residue was then dissolved in dry toluene (2.4 mL) and cooled to 0° C. before slow addition of a 1.5M solution of LiHMDS in THE (0.37 mL, 0.56 mmol) and the reaction mixture was stirred at rt for 1.5 h. A saturated aqueous NH₄Cl solution was added and the reaction mixture was extracted with EtOAc (3×). The combined organic layers were washed with brine, dried over MgSO₄, filtered and concentrated to dryness. The residue was triturated in Et₂O, filtered and vacuum-dried to afford the title compound as an off-white solid (45 mg, yield: 50%). LC-MS (Method B1) m/z [M+H]⁺: 244.1; rt: 0.89 min; purity: 96%. ¹H NMR (400 MHz, DMSO-d₆) δ 10.87 (s, 1H), 8.68 (dd, J=4.7, 1.7 Hz, 1H), 8.18 (d, J=5.5 Hz, 1H), 8.12 (ddd, J=7.9, 4.6, 1.7 Hz, 1H), 7.48 (dd, J=7.9, 4.7 Hz, 1H), 7.18 (d, J=5.5 Hz, 1H), 3.63 (q, J=6.6 Hz, 1H), 1.50 (d, J=6.6 Hz, 3H).

### Step 2: Synthesis of 3-methoxy-10-methyl-4,8,12-triazatricyclo[9.4.0.0^{2,7}]pentadeca-1(11),2(7),3,5,12,14-hexaen-9-one

A suspension of intermediate L14_1 (461 mg, 1.90 mmol) and potassium carbonate (794 mg, 5.69 mmol) in methanol (6.3 mL) was stirred at 70° C. for 4 days. After completion, the reaction mixture was quenched with water (40 mL) and extracted with EtOAc (4×10 mL). The combined organic layers were washed with brine (40 mL), dried over MgSO₄, filtered and concentrated under vacuum to afford the title compound as a white solid (303 mg, yield: 58%). LC-MS (Method A1_S) m/z: [M+H]⁺: 256.1; rt: 0.87 min; purity: 92%. LC-MS (Method B1_S) m/z: [M+H]⁺: 256.1; rt: 0.94 min; purity: 94%. ¹H NMR (400 MHz, DMSO-d₆) δ 10.54 (s, 1H), 8.59 (dd, J=4.7, 1.7 Hz, 1H), 8.15 (dd, J=7.9, 1.7 Hz, 1H), 8.11 (d, J=5.6 Hz, 1H), 7.39 (dd, J=7.9, 4.7 Hz, 1H), 6.86 (d, J=5.6 Hz, 1H), 3.91 (s, 3H), 3.53-3.44 (m, 1H), 1.47 (d, J=6.7 Hz, 3H).

### Intermediate L15: 3-fluoro-5-methoxy-4,8,12-triazatricyclo[9.4.0.0^{2,7}]pentadeca-1(11),2(7),3,5,12,14-hexaen-9-one

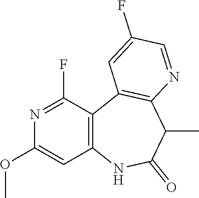 [SMILES: COc1cc2c(c(F)n1)-c1cc(F)cnc1C(C)C(=O)N2]

The title compound was prepared according to the same reaction sequence as the one described for intermediate L9_4 but starting from Intermediate L9_2 and Intermediate L2_3. The first step (Suzuki reaction) was performed with Pd₂dba₃, SPhos and K₃PO₄ in toluene at 100° C. The second step was performed using LiHMDS in THE at RT, both steps similar as those described for intermediate L9_4. LC-MS (Method B1_S) m/z: [M+H]⁺: 292; rt: 1.23 min; purity: 90%. ¹H NMR (400 MHz, DMSO-d₆) δ 10.77 (s, 1H), 8.65 (d, J=2.8 Hz, 1H), 8.06-8.02 (m, 1H), 6.54 (s, 1H), 3.90 (s, 3H), 3.70 (q, J=6.7 Hz, 1H), 1.47 (d, J=6.7 Hz, 3H).

### Intermediate L16: 2-(3,14-difluoro-5,10-dimethyl-9-oxo-4,8,12-triazatricyclo[9.4.0.0^{2,7}]pentadeca-1(11),2(7),3,5,12,14-hexaen-8-yl)acetamide

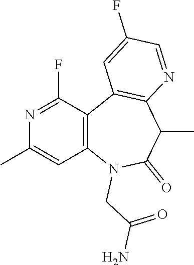

To a solution of Intermediate L2 (300 mg, 1.09 mmol) in DMF (5.5 mL) were added, at room temperature, iodoacetamide (224 mg, 1.20 mmol) and potassium carbonate (183 mg, 1.31 mmol). The resulting mixture was stirred at room temperature for 16 h. After completion, water (15 mL) was added and the reaction mixture was stirred at room temperature for 30 min. The precipitate was filtered over a glass frit, rinsed with water and vacuum-dried to afford the title compound as a white solid (328 mg, yield: 91%). LC-MS (Method A1_S) m/z: [M+H]⁺: 333.1; rt: 0.97 min; purity: 97%. ¹H NMR (400 MHz, DMSO-d₆) δ 8.69 (d, J=2.8 Hz, 1H), 8.09 (ddd, J=9.6, 4.1, 2.8 Hz, 1H), 7.58 (s, 1H), 7.28 (s, 1H), 7.13 (s, 1H), 4.38 (d, J=16.8 Hz, 1H), 4.21 (d, J=16.8 Hz, 1H), 3.75 (q, J=6.6 Hz, 1H), 1.47 (d, J=6.6 Hz, 3H). CH₃ protons under DMSO signal.

### Intermediate L17: (3-oxobenzofuran-6-yl) trifluoromethanesulfonate

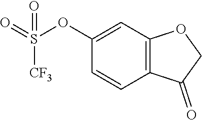 [SMILES: O=C1COc2cc(OS(=O)(=O)C(F)(F)F)ccc21]

To a suspension of 6-hydroxy-2H-benzofuran-3-one (500 mg, 3.33 mmol) in dry DCM (10 mL) at room temperature, was added pyridine (0.82 mL, 10.0 mmol). The mixture was cooled down to 0° C. before addition of trifluoromethanesulfonic anhydride (1 M in DCM, 5.33 mL, 5.33 mmol). The reaction mixture was stirred at 0° C. for 2 h. After completion, water (15 mL) was added and the reaction mixture was extracted with DCM (3×15 mL). The combined organic layers were washed with a 1 M aqueous citric acid solution (2×20 mL), then with a saturated aqueous NaHCO₃ solution (20 mL), brine (20 mL), dried over Na₂SO₄, filtered and concentrated under vacuum. The residue was purified by flash chromatography on silica gel (using a gradient of 0 to 30% EtOAc in Heptane as eluent) to afford the title compound as a yellow solid (601 mg, yield: 61%). LC-MS (Method A1_S) m/z: no mass response; rt: 1.35 min; purity: 96%. ¹H NMR (400 MHz, DMSO-d₆) δ 7.85 (d, J=8.5 Hz, 1H), 7.64 (d, J=2.1 Hz, 1H), 7.25 (dd, J=8.5, 2.1 Hz, 1H), 4.93 (s, 2H).

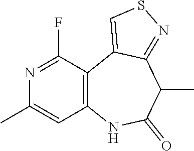

### Step 1: Synthesis of 4-bromo-3-(bromomethyl)isothiazole L18_1

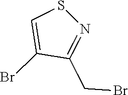

In a vial filled with 4-bromo-3-methylisothiazole (500 mg, 2.68 mmol), N-bromosuccinimide (712 mg, 4.00 mmol) and 1,2-dichloroethane (20 mL) was added 2,2′-azobis(2-methylpropionitrile) (88 mg, 0.54 mmol). The vial was sealed, and the reaction mixture was stirred and heated at 80° C. for 16 h to give a yellow solution. The reaction mixture was quenched with water (30 mL) and extracted two times with EtOAc (2×50 mL). The combined organic layers were separated, dried over anhydrous MgSO₄, and concentrated in vacuo. The crude was purified by column chromatography on silica gel (using 0% to 10% EtOAc in heptane as eluent over 10 CV) to afford the title compound as a pale-yellow oil (660 mg, yield: 96%). LC-MS (Method A1_S) m/z: [M+H]⁺: 257; rt: 1.29 min; purity: 62%.

### Step 2: Synthesis of 2-(4-bromoisothiazol-3-yl)acetonitrile L18_2

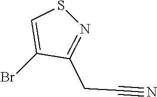

To a solution of intermediate L18_1 (660 mg, 2.5686 mmol) in dry acetonitrile (12 mL) was added trimethylsilyl cyanide (1.0 mL, 7.6 mmol), followed by tetrabutylammonium fluoride (1 mol/L in THF, 3.8 mL) at 0° C. and the reaction was stirred for 16 h at room temperature. The reaction mixture was quenched with water (100 mL) and extracted two times with EtOAc (2×100 mL). The combined organic layers were washed with brine, dried over anhydrous MgSO₄ and concentrated in vacuo to give a brown oil. The crude obtained was purified by column chromatography on silica gel (using 0% to 50% EtOAc in heptane as eluent over 13 CV) to afford the title compound as a colorless oil (200 mg, yield: 38%). LC-MS (Method A1_S) m/z: no mass response; rt: 0.99 min; purity: 100%. ¹H NMR (400 MHz, DMSO-d₆) δ 9.26 (s, 1H), 4.30 (s, 2H).

### Step 3: Synthesis of methyl 2-(4-bromoisothiazol-3-yl)acetate L18_3

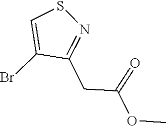

To a solution of intermediate L18_2 (200 mg, 0.98 mmol) in dry methanol (4 mL) was added a solution of hydrochloric acid (4 mol/L in 1,4-dioxane, 1.2 mL) at room temperature and the reaction mixture was heated at 70° C. for 4 days. After completion, methanol was removed under vacuum and the reaction mixture was quenched with a saturated aqueous solution of NaHCO₃ (10 mL) and extracted three times with EtOAc. The combined organic layers were separated, dried over MgSO₄ and concentrated under vacuum. The crude obtained was purified by column chromatography on silica gel (using 5% to 40% EtOAc in heptane as eluent over 7 CV) to give the title compound as a colorless oil (137 mg, yield: 59%). LC-MS (Method A1_S) m/z: [M+H]⁺: 237/239; rt: 1.10 min; purity: 96%.

### Step 4: Synthesis of methyl 2-(4-bromoisothiazol-3-yl)propanoate L18_4

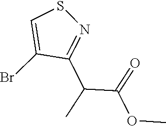

In a dry vial under argon atmosphere, a solution of diisopropylamine (100 μL, 0.70 mmol) in dry THF (0.2 mL) was cooled at 0° C., before N-butyllithium (1.4 mol/L in THF, 0.5 mL) was added dropwise. The reaction mixture was then cooled at −78° C. and stirred for 10 min. A solution of intermediate L18_3 (137 mg, 0.58 mmol) in dry THF (0.3 mL) was added dropwise at −78° C. and the reaction mixture was stirred for 10 min at −78° C. Iodomethane (55 μL, 0.87 mmol) was added and the reaction mixture was allowed to reach room temperature over 30 min. After completion, the reaction mixture was quenched at 0° C. by a saturated solution of NH₄Cl and water and extracted three times with EtOAc. The combined organic layers were washed with brine, dried over MgSO₄, filtered, and concentrated under vacuum. The crude obtained was purified by column chromatography on silica gel (using 0% to 15% EtOAc in heptane as eluent over 10 CV) to give the title compound as a colorless oil (93 mg, yield: 51%). LC-MS (Method A1_S) m/z: [M+H]⁺: 252; rt: 1.24 min; purity: 80%.

### Step 5: Synthesis of 14-fluoro-7,12-dimethyl-4-thia-5,9,13-triazatricyclo[8.4.0.0²′6]tetradeca-1(10),2,5,11,13-pentaen-8-one L18

A mixture of intermediate L1_4 (50 mg, 0.20 mmol), intermediate L18_4 (50 mg, 0.20 mmol) and potassium carbonate (84 mg, 0.60 mmol) in 1,4-dioxane (2 mL) was degassed with nitrogen for 10 min before the addition of XPHOS PD G3 (18 mg, 0.02 mmol). The reaction mixture was heated at 80° C. for 4 h. After completion, the reaction mixture was partitioned between water and a saturated aqueous solution of NH₄Cl and EtOAc. The resulting mixture was extracted with EtOAc. The combined organic extracts were dried with brine, over MgSO₄, filtered off and concentrated under vacuum to give a yellow oil. The crude was dissolved in dry THF (2 mL) and lithium bis(trimethylsilyl)amide (1.5 M in THF, 80 μL) was added at room temperature under N₂ atmosphere. The reaction mixture was stirred for 2 h at room temperature before being poured into water and a saturated aqueous solution of NH₄Cl. The resulting mixture was extracted twice with EtOAc and the combined organic layers were washed with brine. The resulting solution was dried over MgSO₄, filtered, and concentrated to dryness to give a white solid. The residue was triturated with Et₂O, the solid was filtered to afford the title compound (30 mg, yield: 32%). LC-MS (Method A1_S) m/z: [M+H]⁺: 264; rt: 1.06 min; purity: 100%.

### Intermediate L19: 3-fluoro-5-(1-hydroxycyclobutyl)-10-methyl-4,8,12-triazatricyclo[9.4.0.0²,1]pentadeca-1(15),2,4,6,11,13-hexaen-9-one

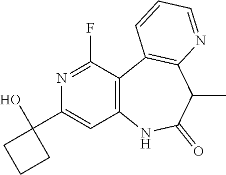 [SMILES: CC1C(=O)Nc2cc(C3(O)CCC3)nc(F)c2-c2cccnc21]

### Step 1: Synthesis of 2-bromo-6-fluoro-pyridin-4-amine L19_1

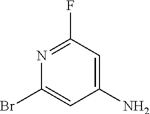

Cesium fluoride (3.65 g, 23.8 mmol) was added to a solution of 2,6-dibromopyridin-4-amine (2.00 g, 7.78 mmol) in dry DMSO (30 mL) at room temperature. The reaction mixture was stirred for 18 h at 140° C. After completion, the reaction mixture was cooled down with ice (100 g) and diluted with water (250 mL) then extracted three times with ethyl acetate (100 mL). The combined organic layers were washed two times with brine (100 mL), dried over Na₂SO₄, filtered, and concentrated to dryness to afford a brown solid. The solution was concentrated under vacuum and the residue was purified by column chromatography on silica gel (using a gradient of 0 to 25% MeOH in DCM) to afford the title compound (773 mg, yield: 28%) as a white solid. LC-MS (Method B4) m/z: [M+H]⁺: 191/193; rt: 0.92 min; purity: 100%. ¹H NMR (400 MHz, DMSO-d₆) δ 6.78 (s, 2H), 6.61 (t, J=1.6 Hz, 1H), 6.08 (d, J=1.6 Hz, 1H). ¹⁹F NMR (376 MHz, DMSO-d₆) δ −71.05.

### Step 2: Synthesis of 2-bromo-6-fluoro-N-trityl-pyridin-4-amine L19_2

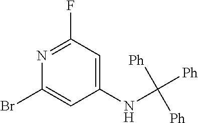 [SMILES: Fc1cc(NC(c2ccccc2)(c2ccccc2)c2ccccc2)cc(Br)n1]

To a solution of intermediate L19_1 (773 mg, 4.18 mmol) and triphenylmethyl chloride (1.25 g, 4.35 mmol) in dichloromethane (8 mL) was added N,N-diisopropylethylamine (765 μL, 4.61 mmol). The reaction mixture was stirred at 40° C. for 48 h. The reaction mixture was concentrated to dryness to afford a yellow foam which was purified by column chromatography on silica gel (using a gradient of 0 to 25% EtOAc in heptane) to afford the title compound (1.18 g, yield: 59%) as a white solid. LC-MS (Method A1_S) m/z: [M+H]⁺: 435/437; rt: 1.76 min; purity: 97%. ¹H NMR (500 MHz, CDCl₃) δ 7.36-7.21 (m, 15H), 6.44 (s, 1H), 5.73 (s, 1H), 5.59 (s, 1H). ¹⁹F NMR (376 MHz, CDCl₃) δ −68.05.

### Step 3: Synthesis of 1-[6-fluoro-4-(tritylamino)-2-pyridyl]cyclobutanol L19_3

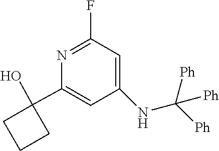 [SMILES: OC1(c2cc(NC(c3ccccc3)(c3ccccc3)c3ccccc3)cc(F)n2)CCC1]

To a solution of intermediate L19_2 (500 mg, 1.15 mmol) in THE (12 mL) at −78° C. was added N-butyllithium (1.5 mol/L in hexanes, 2.89 mL) and the reaction mixture was stirred at −78° C. for 1 h. After 1 h, cyclobutanone (195 μL, 2.55 mmol) was added at −78° C. and the reaction mixture was slowly allowed to reach room temperature. After 18 h, EtOAc (30 mL) and water (30 mL) were added, and the layers were separated. The aqueous layer was extracted with EtOAc twice. The combined organic layers were washed three times with water and brine. The resulting solution was dried over MgSO₄, filtered, and concentrated to dryness to give a yellow oil. The residue was purified by column chromatography on silica gel (using a gradient of 0 to 60% EtOAc in heptane) to afford the title compound (206 mg, yield: 34%) as a white solid. LC-MS (Method A1_S) m/z: [M+H]⁺: 425; rt: 1.76 min; purity: 85%. ¹H NMR (500 MHz, CDCl₃) δ 7.35-7.23 (m, 15H), 6.32 (s, 1H), 5.74 (s, 1H), 5.69 (d, J=10.8 Hz, 1H), 4.21 (s, 1H), 2.29 (m, 2H), 2.16-2.07 (m, 2H), 1.69-1.52 (m, 1H), 1.79 (m, 1H).

### Step 4: Synthesis of 1-(4-amino-6-fluoro-2-pyridyl)cyclobutanol L19_4

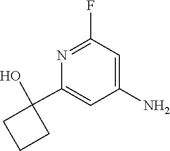 [SMILES: Nc1cc(F)nc(C2(O)CCC2)c1]

To a solution of intermediate L19_3 (206 mg, 0.39 mmol) in 1,4-dioxane (4 mL) was added hydrochloric acid (4 mol/L in 1,4-dioxane, 924 μL) and the reaction mixture was stirred at 60° C. After 6 h, the reaction mixture was concentrated under vacuum to afford the title compound (98 mg, yield: 89%) as a brown oil.

LC-MS (Method A1_S) m/z: [M+H]⁺: 183; rt: 0.67 min; purity: 90%.

¹H NMR (500 MHz, CDCl₃) δ 6.63 (t, J=1.8 Hz, 1H), 5.99 (d, J=1.8 Hz, 1H), 4.39 (s, 2H), 4.03 (s, 1H), 2.50-2.37 (m, 4H), 2.04-1.97 (m, 1H), 1.80-1.75 (m, 1H).

¹⁹F NMR (376 MHz, CDC) δ −70.50.

### Step 5: Synthesis of 1-(4-amino-5-bromo-6-fluoro-2-pyridyl)cyclobutanol L19_5

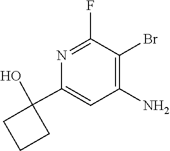 [SMILES: Nc1cc(C2(O)CCC2)nc(F)c1Br]

To a solution of intermediate L19_4 (98 mg, 0.20 mmol) in acetonitrile (0.2 mL) at 0° C. was slowly added N-bromosuccinimide (40 mg, 0.23 mmol). The resulting mixture was stirred at 0° C. for 30 min. The resulting mixture was concentrated under vacuum to afford an orange oil. The residue was purified by preparative HPLC (Purification Method P_A) to afford the title compound (108 mg, yield: 98%) as a white solid. LC-MS (Method B4) m/z: [M+H]⁺: 243/245; rt: 3.14 min; purity: 100%. ¹H NMR (400 MHz, CDCl₃) δ 6.73 (s, 1H), 4.89 (s, 2H), 3.79-3.53 (m, 1H), 2.51-2.44 (m, 2H), 2.44-2.32 (m, 2H), 2.07-1.96 (m, 1H), 1.86-1.75 (m, 1H). ¹⁹F NMR (376 MHz, CDCl₃) δ −69.68.

### Step 6: Synthesis of 3-fluoro-5-(1-hydroxycyclobutyl)-10-methyl-4,8,12-triazatricyclo[9.4.0.0^{2,7}]pentadeca-1(15),2,4,6,11,13-hexaen-9-one L19

A mixture of intermediate L5_3 (152 mg, 0.50 mmol), intermediate L19_5 (108 mg, 0.41 mmol), potassium phosphate tribasic (179 mg, 0.83 mmol) and 2-dicyclohexylphosphino-2,6-dimethoxybiphenyl (17 mg, 0.04 mmol) in 1,4-dioxane (2 mL) and water (124 μL) was degassed with nitrogen for 10 min before addition of tris(dibenzylideneacetone)dipalladium(0) (38 mg, 0.04 mmol). The reaction mixture was heated at 100° C. for 16 h. EtOAc (5 mL) and Water (5 mL) were added to the reaction mixture. The aqueous layer was extracted three times with EtOAc. The combined organic layers were washed with brine, dried over MgSO₄, filtered, and concentrated to dryness to afford a black oil. The crude was dissolved in dry THE (0.8 mL) and lithium bis(trimethylsilyl)amide (1.5 M in THF, 34 μL) was added at room temperature under N₂ atmosphere. The reaction mixture was stirred for 2 h at room temperature. After 2 h, water and ethyl acetate were added, and the layers were separated. The aqueous layer was extracted with EtOAc twice. The combined organic layers were washed three times with water and brine. The resulting solution was dried over MgSO₄, filtered, and concentrated to dryness to give a yellow oil. The residue was purified by column chromatography on silica gel (using a gradient of 0 to 100% EtOAc in heptane) to afford the title compound (32 mg, yield: 95%). LC-MS (Method B1_S) m/z: [M+H]⁺: 314; rt: 1.06 min; purity: 96.5%. ¹H NMR (500 MHz, CDCl₃) δ 8.72 (dd, J=4.7, 1.7 Hz, 1H), 8.18 (s, 1H), 8.04 (ddd, J=7.9, 4.7, 1.7 Hz, 1H), 7.37 (dd, J=7.9, 4.7 Hz, 1H), 7.21 (s, 1H), 5.30 (s, 1H), 3.57 (q, J=6.5 Hz, 1H), 2.70-2.58 (m, 2H), 2.50-2.39 (m, 2H), 2.19-2.05 (m, 1H), 2.02-1.89 (m, 1H), 1.73 (d, J=6.5 Hz, 3H). ¹⁹F NMR (471 MHz, CDCl₃) δ −68.50 (d, J=4.8 Hz).

### Intermediate L20: 3,14-difluoro-5-(hydroxymethyl)-10-methyl-4,8,12-triazatricyclo[9.4.0.0^{2,7}]pentadeca-1(11),2,4,6,12,14-hexaen-9-one

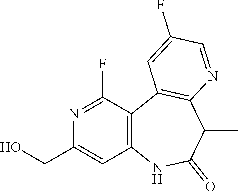 [SMILES: CC1C(=O)Nc2cc(CO)nc(F)c2-c2cc(F)cnc21]

### Step 1: Synthesis of tert-butyl N-(2-bromo-6-fluoro-4-pyridyl)-N-tert-butoxycarbonyl-carbamate L20_1

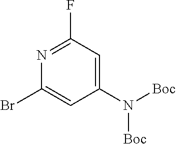 [SMILES: CC(C)(C)OC(=O)N(C(=O)OC(C)(C)C)c1cc(F)nc(Br)c1]

To a solution of Intermediate L19_1 (700 mg, 3.40 mmol) and di-tert-butyl decarbonate (2.30 g, 10 mmol) in THE (30 mL) was added 4-dimethylaminopyridine (43 mg, 0.35 mmol). The resulting mixture was stirred at room temperature for 16 h. After completion, water was added, and the reaction mixture was extracted three times with EtOAc. The combined organic layers were washed with brine, dried over Na₂SO₄, filtered, and concentrated to dryness afford an orange oil. The residue was purified by column chromatography on silica gel (using a gradient of 0 to 20% EtOAc in heptane over 10 CV) to afford the title compound (1.13 g, yield: 78%) as a white solid. LC-MS (Method A1_S) m/z: [M+H]⁺: 391/393; rt: 1.70 min; purity: 98%. ¹H NMR (400 MHz, DMSO-d₆) δ 7.72 (s, 1H), 7.38 (s, 1H), 1.42 (s, 18H).

### Step 2: Synthesis of tert-butyl N-[2-fluoro-6-(hydroxymethyl)-4-pyridyl]carbamate L20_2

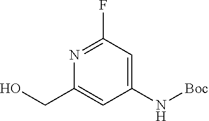 [SMILES: CC(C)(C)OC(=O)Nc1cc(F)nc(CO)c1]

To a solution of intermediate L20_1 (781 mg, 2.00 mmol), hexakis(p-acetate)thiopalladium(II) (269 mg, 0.40 mmol) and butyldi-1-adamantylphosphine (301 mg, 0.80 mmol) in N,N-dimethylformamide (3.3 mL) was added N,N,N′,N′-tetramethylethylenediamine (466 mg, 4.00 mmol). The reaction mixture was placed under 5 bar of syngas at 100° C. during 16 h. After completion, the reaction mixture was filtered over celite. The filtrate was extracted with EtOAc and washed three times with brine, dried over MgSO₄, filtered, and concentrated under vacuum to give a black oil. The residue was dissolved in methanol (18 mL) at 0° C. before addition of sodium borohydride (132 mg, 3.31 mmol). The reaction mixture was stirred at room temperature for 4 h. After completion, the reaction mixture was quenched with water and extracted three times with EtOAc. The resulting mixture was dried over MgSO₄, filtered, and concentrated under vacuum. The residue was purified by preparative HPLC (Purification Method P_A) to afford the title compound as a white solid (163 mg, yield: 37%). LC-MS (Method A1_S) m/z: [M+H-Boc]⁺: 143; rt: 1.24 min; purity: 98%. ¹H NMR (500 MHz, DMSO-d₆) δ 10.14 (s, 1H), 7.45 (s, 1H), 7.00 (d, J=1.6 Hz, 1H), 5.48 (s, 1H), 4.38 (s, 2H), 1.48 (s, 9H).

### Step 3: Synthesis of (4-amino-6-fluoro-2-pyridyl)methanol L20_3

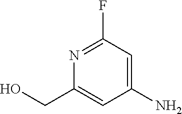

To a solution of intermediate L20_2 (163 mg, 0.65 mmol) in dichloromethane (6.5 mL) at room temperature was added trifluoroacetic acid (490 μL, 6.53 mmol). The reaction mixture was stirred at room temperature for 18 hours. After completion, the reaction mixture was concentrated under vacuum. The residue was dissolved in water and DCM, extracted three times with DCM, dried over MgSO₄, filtered, and concentrated under vacuum to afford the title compound (56 mg, yield: 48%) as a brown oil. LC-MS (Method A1_S) m/z: [M+H]⁺: 143; rt: 0.35 min; purity: 94%.

### Step 4: Synthesis of (4-amino-5-bromo-6-fluoro-2-pyridyl)methanol L20_4

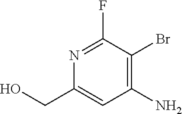 [SMILES: Nc1cc(CO)nc(F)c1Br]

To a solution of intermediate L20_3 (56 mg, 0.27 mmol) in acetonitrile (0.2 mL) at 0° C. was slowly added N-bromosuccinimide (20 mg, 0.11 mmol). The resulting mixture was stirred at room temperature for 15 min. The resulting mixture was concentrated under vacuum to afford a brown oil. The residue was dissolved in DCM and water, extracted with DCM, dried over MgSO₄, filtered, and concentrated under vacuum to afford the title compound as a white solid (45 mg, yield: 67%) LC-MS (Method A1_S) m/z: [M+H]⁺: 222/224; rt: 0.81 min; purity: 98%. ¹H NMR (500 MHz, CDCl₃) δ 6.53 (s, 1H), 4.84 (s, 2H), 4.55 (s, 2H), 2.78 (s, 1H).

### Step 5: Synthesis of 3,14-difluoro-5-(hydroxymethyl)-10-methyl-4,8,12-triazatricyclo[9.4.0.0^{2,7}]pentadeca-1(11),2,4,6,12,14-hexaen-9-one L20

To a solution of intermediate L20_4 (35 mg, 0.16 mmol), intermediate L4_2 (176 mg, 0.19 mmol) in 1,4-dioxane (0.8 mL) and water (48 μl) were added 2-dicyclohexylphosphino-2,6-dimethoxybiphenyl (7 mg, 0.02 mmol) and potassium phosphate tribasic (69 mg, 0.32 mmol). The reaction mixture was degassed with nitrogen for 10 min before addition of tris(dibenzylideneacetone)dipalladium(0) (15 mg, 0.02 mmol). The reaction mixture was heated at 100° C. for 18 h. After completion, EtOAc (5 mL) and water (5 mL) were added in the reaction mixture. The aqueous layer was extracted three times with EtOAc. The combined organic layers were washed with brine, dried over MgSO₄, filtered, and concentrated to dryness to afford a brown oil. The crude oil was dissolved in dry THE (0.7 mL) and lithium bis(trimethylsilyl)amide (1.5 M in THF, 25 μL) was added under N₂ atmosphere. The reaction mixture was concentrated under vacuum and the residue was purified by column chromatography on silica gel (using a gradient of 0 to 100% EtOAc in heptane) to afford the title compound (19 mg, yield: 92%) as a yellow oil. LC-MS-Acid (Method A1_S) m/z: [M+H]⁺: 290; rt: 0.92 min; purity: 94%.

### Intermediate L21: 5-cyclobutyl-3-fluoro-10-methyl-4,8,12-triazatricyclo[9.4.0.0^{2,7}]pentadeca-1(15),2,4,6,11,13-hexaen-9-one

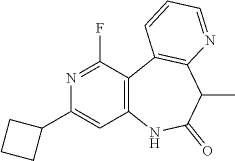 [SMILES: CC1C(=O)Nc2cc(C3CCC3)nc(F)c2-c2cccnc21]

### Step 1: Synthesis of 2-cyclobutyl-6-fluoro-pyridin-4-amine L21_1

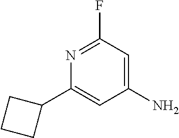 [SMILES: Nc1cc(F)nc(C2CCC2)c1]

Under argon atmosphere, to a mixture of Intermediate L19_1 (650 mg, 3.40 mmol), [1,1′-bis(diphenylphosphino)ferrocene]dichloropalladium(II) (524 mg, 0.68 mmol) and cuprous iodide (129 mg, 0.68 mmol) in 1,4-dioxane (34 mL) at room temperature, was added cyclobutylzinc bromide (20 mL, 0.5 M in THF). The reaction mixture was then stirred at 80° C. After 1 h, the reaction mixture was quenched with ice and saturated aqueous solution of NH₄Cl and then, extracted with ethyl acetate. The combined organic layers were washed with brine, dried over Na₂SO₄, filtered, and concentrated to dryness to afford a black oil. The residue was purified by column chromatography on silica gel (using a gradient of 0 to 15% EtOAc in heptane) to afford the title compound (630 mg, yield: 100%) as a red oil. LC-MS (Method A1_S) m/z: [M+H]⁺: 167; rt: 0.91 min; purity: 90.1%. ¹H NMR (400 MHz, CDCl₃) δ 6.26 (d, J=1.9 Hz, 1H), 5.91 (s, 1H), 4.27 (s, 2H), 3.45 (p, J=8.7 Hz, 1H), 2.32-2.19 (m, 4H), 2.06-1.91 (m, 1H), 1.90-1.77 (m, 1H). ¹⁹F NMR (376 MHz, CDCl₃) δ −69.87.

### Step 3: Synthesis of 3-bromo-6-cyclobutyl-2-fluoro-pyridin-4-amine L21_2

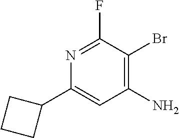 [SMILES: Nc1cc(C2CCC2)nc(F)c1Br]

To a solution of intermediate L21_1 (860 mg, 4.14 mmol) in acetonitrile (20 mL) at 0° C. was slowly added N-bromosuccinimide (736 mg, 4.14 mmol). The resulting mixture was stirred at 0° C. for 10 min. The reaction mixture was treated with a saturated aqueous solution of NaHCO₃ and extracted with ethyl acetate. The organic layer was washed with water, brine, dried over Na₂SO₄, filtered, and concentrated under vacuum to afford a black oil. The residue was purified by column chromatography on silica gel (using a gradient of 0 to 20% EtOAc in heptane) to afford the title compound (459 mg, yield: 43%) as a white solid. LC-MS (Method A1_S) m/z: [M+H]⁺: 245/247; rt: 1.38 min; purity: 96.3%. ¹H NMR (500 MHz, DMSO-d₆) δ 6.59 (s, 2H), 6.41 (s, 1H), 3.46-3.25 (m, 1H), 2.22-2.05 (m, 4H), 1.98-1.86 (m, 1H), 1.82-1.71 (m, 1H).

### Step 4: 5-cyclobutyl-3-fluoro-10-methyl-4,8,12-triazatricyclo[9.4.0.0^{2,7}]pentadeca-1(15),2,4,6,11,13-hexaen-9-one L21

A mixture of Intermediate L5_3 (800 mg, 2.62 mmol), Intermediate L21_2 (460 mg, 1.79 mmol), cesium fluoride (800 mg, 5.26 mmol) and bis(di-tert-butyl(4-dimethylaminophenyl)phosphine)dichloropalladium(II) (90 mg, 0.13 mmol) in 1,4-dioxane (15 mL) and water (1.3 mL) was degassed with nitrogen for 10 min. The reaction mixture was heated at 80° C. for 16 h. The reaction mixture was diluted with saturated aqueous of NH₄Cl and extracted with ethyl acetate. The organic layer was washed with brine, dried over Na₂SO₄, filtered, and concentrated to afford a brown oil. The crude was dissolved in dry THE (10 mL) and lithium bis(trimethylsilyl)amide (1.5 M in THF, 2.5 mL) was added at room temperature under N₂ atmosphere. The reaction mixture was stirred for 1 h at room temperature. After 1 h, water and ethyl acetate were added, and the layers were separated. The aqueous layer was extracted with EtOAc twice. The combined organic layers were washed three times with water and brine. The resulting solution was dried over MgSO₄, filtered, and concentrated to dryness to give a brown solid. The residue was purified by column chromatography on silica gel (using a gradient of 0 to 13% MeOH in DCM) to afford the title compound (260 mg, yield: 41%). LC-MS (Method A1_S) m/z: [M+H]⁺: 298; rt: 1.31 min; purity: 83.9%.

### Intermediate L22: 2-fluoro-5,9-dimethyl-6-oxo-6,7-dihydro-5H-benzo[b]pyrido[3,2-d]azepine-10-carbonitrile

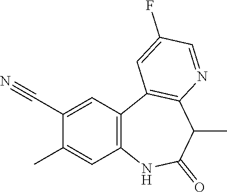 [SMILES: Cc1cc2c(cc1C#N)-c1cc(F)cnc1C(C)C(=O)N2]

### Step 1: Synthesis of 4-amino-2-methyl-5-(4,4,5,5-tetramethyl-1,3,2-dioxaborolan-2-yl)benzonitrile L221

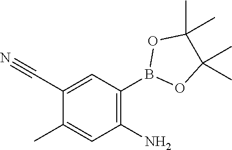 [SMILES: Cc1cc(N)c(B2OC(C)(C)C(C)(C)O2)cc1C#N]

To a solution of 4-amino-2-methyl-benzonitrile (97.0%, 1.00 g, 7.34 mmol) in THF (60 mL) was added 4,4,5,5-tetramethyl-1,3,2-dioxaborolane (2.1 mL, 14.7 mmol) and the resulting solution was stirred for 30 min. Then, (1,5-cyclooctadiene)(methoxy)iridium(i) dimer (243 mg, 0.37 mmol) and 2-[4-(dimethylamino)-2-pyridyl]-N,N-dimethyl-pyridin-4-amine (178 mg, 0.73 mmol) were added sequentially and the resulting mixture was purged with nitrogen gas for 5 min and stirred at 80° C. for 18 h. The reaction mixture was filtered through a pad of Celite, which was washed with EtOAc (150 mL). The filtrate was concentrated, and the resulting residue was purified by column chromatography on silica gel (using a gradient of 0-70% EtOAc/iso-hexane as eluent) to afford the title compound as a white solid (892 mg, yield: 47%). LC-MS (Method B5) m/z [M+H]⁺: 259.2; rt: 2.02 min; purity: 100%. ¹H NMR (400 MHz, DMSO-d₆) δ 7.56 (s, 1H), 6.56 (d, J=1.0 Hz, 1H), 6.28 (s, 2H), 2.29 (s, 3H), 1.29 (s, 12H).

### Step 2: Synthesis of 2-fluoro-5,9-dimethyl-6-oxo-5,7-dihydropyrido[2,3-d][1]benzazepine-10-carbonitrile L22

To a mixture of Intermediate L22_1 (531 mg, 2.06 mmol), Intermediate L2_3 (500 mg, 1.53 mmol) and CsF (464 mg, 3.05 mmol) in 1,4-dioxane (25 mL) and water (1.5 mL) was added bis(di-tert-butyl(4-dimethylaminophenyl)phosphine)dichloropalladium(II) (54 mg, 0.08 mmol). The reaction mixture was stirred at 90° C. for 18 h, then concentrated under vacuum. The residue was purified by column chromatography on silica gel (using a gradient of 10% MeOH (0.7M NH₃)/DCM as eluent) to afford the title compound (250 mg, yield: 52%) as an off-white solid. LC-MS (Method B5) m/z [M+H]⁺: 282.0; rt: 1.22 min; purity: 82%. ¹H NMR (400 MHz, DMSO-d₆) δ 10.65 (s, 1H), 8.67 (d, J=2.7 Hz, 1H), 8.22 (s, 1H), 8.12 (dd, J=9.7, 2.8 Hz, 1H), 7.24 (s, 1H), 3.49 (q, J=6.5 Hz, 1H), 2.53 (s, 3H), 1.47 (d, J=6.5 Hz, 3H). 19F NMR (376 MHz, DMSO-d₆) δ −130.12. purity: 90%.

### Intermediate L23: 3,5-difluoro-4,8,12-triazatricyclo[9.4.0.0^{2,7}]pentadeca-1(11),2,4,6,12,14-hexaen-9-one

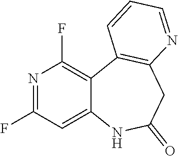 [SMILES: O=C1Cc2ncccc2-c2c(cc(F)nc2F)N1]

The title compound was prepared according to the same reaction sequence as the one described for intermediate L6_2 starting from Intermediate L6_1 and Intermediate L5_1. The first step (Suzuki reaction) was performed with CsF, PEPPSI-Ipent heated in H₂O/toluene at 80° C. The second step was performed using LiHMDS (3 eq.) in toluene at RT (17% yield over 2 steps), both steps similar as those described for intermediate L6_2. LC-MS (Method A7) m/z [M+H]⁺: 248.1; rt: 1.32 min. ¹H NMR (400 MHz, DMSO-d₆) δ 11.04 (s, 1H), 8.62 (dd, J=4.8, 1.7 Hz, 1H), 8.13 (ddd, J=8.0, 4.8, 1.7 Hz, 1H), 7.50 (dd, J=8.0, 4.8 Hz, 1H), 6.88 (s, 1H), 3.92 (d, J=12.7 Hz, 1H), 3.67 (d, J=12.7 Hz, 1H).

### Intermediate L24: 3-fluoro-5-methoxy-4,8,12-triazatricyclo[9.4.0.0^{2,7}]pentadeca-1(11),2(7),3,5,12,14-hexaen-9-one

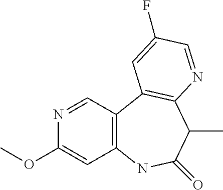

### Step 1: Synthesis of 2-methoxy-5-(4,4,5,5-tetramethyl-1,3,2-dioxaborolan-2-yl)pyridin-4-amine L24_1

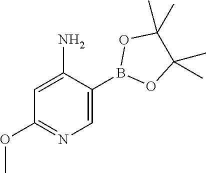 [SMILES: COc1cc(N)c(B2OC(C)(C)C(C)(C)O2)cn1]

To a flame-dried 100 mL, 3-neck round-bottom flask under nitrogen were added 4-amino-2-methoxypyridine (1.0 g, 7.8 mmol), bis(pinacolato)diboron (4.0 g, 16 mmol), (1,5-cyclooctadiene)(methoxy)iridium(I) dimer (100 mg, 0.15 mmol) and 4,4′-di-tert-butyl-2,2′-dipyridyl (90 mg, 0.32 mmol). The flask was evacuated and back-filled with nitrogen three times prior to the addition of anhydrous 1,4-dioxane (20 mL). The reaction mixture was stirred at 75° C. (internal temperature) for 17 hours before it was cooled down to RT. The solution mixture was used as such in the next step with cautious care (gas evolution in the next step).

### Step 2: 3-fluoro-5-methoxy-4,8,12-triazatricyclo[9.4.0.0²⁷]pentadeca-1(11),2(7),3,5,12,14-hexaen-9-one L24

The title compound was prepared according to the same reaction sequence as the one described for intermediate L3 starting from intermediate L24_1 and Intermediate L2_3. The first step (Suzuki reaction) was performed with Pd₂dba₃, SPhos and K₃PO₄ in toluene at 100° C. The second step was performed using LiHMDS in THE at RT, both steps similar as those described for intermediate L3. LC-MS (Method B1_S) m/z [M+H]⁺: 274; rt: 1.04 min. ¹H NMR (500 MHz, DMSO-d₆) δ 10.61 (s, 1H), 8.63 (d, J=2.8 Hz, 1H), 8.54 (s, 1H), 8.07 (dd, J=9.7, 2.8 Hz, 1H), 6.57 (s, 1H), 3.92 (s, 3H), 3.57 (q, J=6.6 Hz, 1H), 1.46 (d, J=6.6 Hz, 3H).

### Intermediate L25: 11-fluoro-9-(3-fluoroazetidin-1-yl)-5-methyl-5,7-dihydropyrido[2,3-d][1]benzazepin-6-one

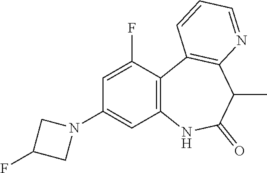 [SMILES: CC1C(=O)Nc2cc(N3CC(F)C3)cc(F)c2-c2cccnc21]

### Step 1: Synthesis of 5-chloro-3-fluoro-2-(4,4,5,5-tetramethyl-1,3,2-dioxaborolan-2-yl)aniline L25_1

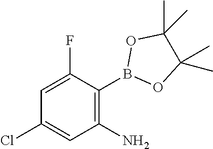 [SMILES: CC1(C)OB(c2c(N)cc(Cl)cc2F)OC1(C)C]

A flame-dried 250 mL, 3-neck, round-bottom flask under nitrogen was charged with bis(pinacolato)diboron (15 g, 58 mmol), (1,5-cyclooctadiene)(methoxy)iridium(I) dimer (190 mg, 0.28 mmol) and 4,4′-di-tert-butyl-2,2′-bipyridyl (160 mg, 0.58 mmol). The flask was evacuated and backfilled with nitrogen three times prior to the addition of a solution of 3-chloro-5-fluoroaniline (4.3 g, 29 mmol) in anhydrous tetrahydrofuran (60 mL) under nitrogen. The stirred mixture was heated at 65° C. (internal temperature) for 19 hours before it was cooled to RT. Methanol (30 mL) was added carefully to the cooled reaction mixture and the resulting solution stirred for 30 minutes at room temperature and then concentrated under vacuo. Purification by flash chromatography (Biotage Selekt, Sfar Silica HCD, 100 g, 0-20% ethyl acetate in hexane) afforded 5-chloro-3-fluoro-2-(4,4,5,5-tetramethyl-1,3,2-dioxaborolan-2-yl)aniline (6.60 g, 23 mmol, 80% Yield) an off-white solid. LC-MS m/z: 272.0 [M+H]+; purity: 96%. ¹H NMR (300 MHz, DMSO-d₆) δ 6.53-6.44 (m, 1H), 6.28 (dd, J=9.7, 1.9 Hz, 1H), 6.11 (s, 2H), 1.28 (s, 12H).

### Step 2: Synthesis of 9-chloro-11-fluoro-5-methyl-5,7-dihydropyrido[2,3-d][1]benzazepin-6-one L25_2

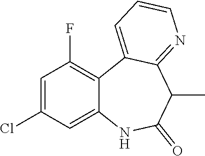 [SMILES: CC1C(=O)Nc2cc(Cl)cc(F)c2-c2cccnc21]

The title compound was prepared according to the same reaction sequence as the one described for intermediate L3 starting from Intermediate L25_1, methyl 2-(3-bromopyridin-2-yl)propanoate, CsF, bis(di-tert-butyl(4-dimethylaminophenyl)phosphine)dichloropalladium(II) heated in dioxane/water at 80° C. followed by cyclization with LiHMDS in THE at 0° C. (45% yield). LC-MS m/z: 277.0/279.0 [M+H]+; purity: 100%. ¹H NMR (300 MHz, DMSO-d₆) δ 10.47 (s, 1H), 8.66 (dd, J=4.8, 1.7 Hz, 1H), 8.04 (ddd, J=7.9, 4.6, 1.7 Hz, 1H), 7.52-7.38 (m, 2H), 7.16 (dd, J=2.1, 1.5 Hz, 1H), 3.58 (q, J=6.6 Hz, 1H), 1.47 (d, J=6.7 Hz, 3H). 19F NMR (282 MHz, DMSO-d₆) δ −112.89 (ddd, J=10.5, 4.6, 1.5 Hz.

### Step 3: Synthesis of 11-fluoro-9-(3-fluoroazetidin-1-yl)-5-methyl-5,7-dihydropyrido[2,3-d][1]benzazepin-6-one

A mixture of Intermediate L25_2 (100 mg, 0.36 mmol) and 3-fluoroazetidine hydrochloride (60 mg, 0.51 mmol) in 1,4-dioxane (3 mL) was added (2-Dicyclohexylphosphino-2′,6′-diisopropoxy-1,1′-biphenyl)[2-(2′-amino-1,1′-biphenyl)]palladium(II) methanesulfonate (30 mg, 0.04 mmol) followed by 2-Dicyclohexylphosphino-2′,6′-diisopropoxybiphenyl (17 mg, 0.04 mmol). The vial was sonicated and flushed under argon, before the addition of sodium tert-butoxide (105 mg, 1.09 mmol). The vial was sealed, and the reaction mixture was heated at 90° C. for 4 h. After completion, water and EtOAc were added. The combined organic layers were washed with brine, separated, dried over MgSO₄, filtered, and concentrated under vacuum. The residue was triturated with DCM to afford the title compound (68 mg, yield: 60%) as a white solid. LC-MS (Method A1_S) m/z: [M+H]⁺: 316.1; rt: 1.09 min; purity: 90%.

### Intermediate L26: 14-chloro-4-fluoro-7,12-dimethyl-5,6,9,13-tetrazatricyclo[8.4.0.0^{2,7}]tetradeca-1(14),2,4,10,12-pentaen-8-one

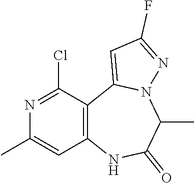

### Step 1: Synthesis of 3-fluoro-1-tetrahydropyran-2-yl-5-(4,4,5,5-tetramethyl-1,3,2-dioxaborolan-2-yl)pyrazole L26_1

To a solution of 3-fluoro-1-tetrahydropyran-2-yl-pyrazole (4.61 g, 26.5 mmol) (CAS 2200261-28-3) in THE (100 mL) at −70° C. was added a solution of n-BuLi in hexanes (2.5 M, 12.7 mL, 31.9 mmol) dropwise. The reaction mixture was stirred at −70° C. for 1 h. Triisopropyl borate (7.4 mL, 31.9 mmol) was added at −78° C. and the reaction mixture was stirred at this temperature for 30 minutes, then allowed to warm to RT and stirred for 1 h. Pinacol (3.76 g, 31.9 mmol) and AcOH (3.1 mL, 54.5 mmol) were added and the reaction mixture was stirred overnight at RT. Water (100 mL) was added and the aqueous phase was extracted with EtOAc (3×75 mL). The combined organics were washed with brine (100 mL), dried over Na₂SO₄, filtered, and concentrated under vacuum. The crude was purified by column chromatography on silica gel (using a gradient of 0-100% TBME in isohexane as eluant) to give the title compound as an off-white solid (7.3 g, yield: 84%). LC-MS m/z [M-166+H]+: 131.2; purity: 98% (215 nm). ¹H NMR (400 MHz, CDCl₃) δ 6.22 (d, J=6.0 Hz, 1H), 5.70 (dt, J=10.0, 2.6 Hz, 1H), 4.08-4.01 (m, 1H), 3.64 (td, J=11.3, 2.8 Hz, 1H), 2.40-2.25 (m, 1H), 2.08 (d, J=14.5 Hz, 1H), 1.91 (dd, J=13.5, 3.5 Hz, 1H), 1.68 (qt, J=11.8, 7.9 Hz, 2H), 1.62-1.49 (m, 1H), 1.34 (s, 12H).

### Step 2: Synthesis of 2-chloro-3-(5-fluoro-2-tetrahydropyran-2-yl-pyrazol-3-yl)-6-methyl-pyridin-4-amine L26_2

Nitrogen was sparged through a solution of 3-fluoro-1-tetrahydropyran-2-yl-5-(4,4,5,5-tetramethyl-1,3,2-dioxaborolan-2-yl)pyrazole L26_1 (1.0 g, 3.1 mmol), 3-bromo-2-chloro-6-methyl-pyridin-4-amine L4_1 (1.0 g, 4.7 mmol), CsF (951 mg, 6.26 mmol) and bis(di-tert-butyl(4-dimethylaminophenyl)phosphine)dichloropalladium(II) (332 mg, 0.470 mmol) in 1,4-dioxane (35 mL) and water (7 mL). The reaction mixture was heated at reflux overnight then cooled to RT. The solution was filtered over a pad of celite and washed through with EtOAc (150 mL). The filtrate was concentrated under vacuum. The crude was purified by column chromatography on silica gel (using a gradient of 0-100% EtOAc in isohexane as eluant) to afford the title compound as a yellow wax (950 mg, yield: 59%). LC-MS m/z [M+H]⁺: 311.2, 313.2; rt: 0.79 and 0.92 min (as a mixture of diastereoisomers); purity: 96%.

### Step 3: Synthesis of 2-chloro-N-[2-chloro-3-(5-fluoro-2-tetrahydropyran-2-yl-pyrazol-3-yl)-6-methyl-4-pyridyl]propenamide L26_3

To a solution of 2-chloro-3-(5-fluoro-2-tetrahydropyran-2-yl-pyrazol-3-yl)-6-methyl-pyridin-4-amine L26_2 (1.05 g, 2.0 mmol) in DMF (18 mL) at 0° C. was added NaH (60% purity, 97.3 mg, 2.4 mmol). The solution was stirred for 30 minutes then 2-chloropropanoyl chloride (0.28 mL, 2.84 mmol) was added dropwise. The reaction mixture was stirred for 1 h at 0° C., then allowed to RT and stirred for 1 h. Water (50 mL) was added and the aqueous phase was extracted with EtOAc (3×50 mL). The combined organic extracts were washed with brine (100 mL), dried over Na₂SO₄, filtered and concentrated under reduced pressure. The crude was purified by column chromatography on silica gel (using a gradient of 0-100% EtOAc in isohexane as eluant) to afford the title compound as a yellow oil (183 mg, yield: 21%). LC-MS m/z [M+H]⁺: 401.0, 403.0; rt: 1.16 and 2.00 min (as a mixture of diastereoisomers); purity: 94%.

### Step 4: Synthesis of 13-chloro-7-(1-chloroethyl)-4-fluoro-11-methyl-5,6,8,12-tetrazatricyclo[7.4.0.0^{2,6}]trideca-1(13),2,4,7,9,11-hexaene L26_4

A solution of 2-chloro-N-[2-chloro-3-(5-fluoro-2-tetrahydropyran-2-yl-pyrazol-3-yl)-6-methyl-4-pyridyl]propanamide #389_3 (288 mg, 0.703 mmol) and HCl (4 M in 1,4-dioxane, 2.0 mL, 8.0 mmol) was stirred at RT overnight. The reaction mixture was evaporated to dryness to afford the title compound as an off-white solid (155 mg, yield: 64%). LC-MS m/z [M+H]⁺: 299.1, 301.1; purity: 96%. ¹H NMR (400 MHz, DMSO-d₆) δ 7.82 (s, 1H), 7.48 (d, J=5.2 Hz, 1H), 5.93 (q, J=6.7 Hz, 1H), 2.64 (s, 3H), 1.97 (d, J=6.7 Hz, 3H).

### Step 5: Synthesis of 14-chloro-4-fluoro-7,12-dimethyl-5,6,9,13-tetrazatricyclo[8.4.0.0² 6]tetradeca-1(14),2,4,10,12-pentaen-8-one L26

To a suspension of 13-chloro-7-(1-chloroethyl)-4-fluoro-11-methyl-5,6,8,12-tetrazatricyclo[7.4.0.0^{2,6}]trideca-1(13),2,4,7,9,11-hexaene L26_4 (155 mg, 0.508 mmol) in DMSO (2.00 mL) was added aqueous NaOH (2 M, 0.51 mL, 1.0 mmol). The resulting orange solution was stirred at RT for 1 h, then water (20 mL) was added followed by AcOH (0.5 mL). The resulting suspension was filtered and the solid was washed with water and dried under vacuum to afford the title compound as an off-white solid (120 mg, yield: 83%). LC-MS m/z [M+H]⁺: 281.1, 283.1; purity: 100%. ¹H NMR (400 MHz, DMSO-d₆) δ 11.06 (s, 1H), 7.06 (s, 1H), 6.58 (d, J=5.8 Hz, 1H), 4.87 (d, J=8.1 Hz, 1H), 2.54 (s, 3H), 1.58 (s, 3H).

### IV. Examples

### Example #1: N-(4-acetylphenyl)-2-[(10R)-3-fluoro-5,10-dimethyl-9-oxo-4,8,12-triazatricyclo[9.4.0.0^{2,7}]pentadeca-1(11),2,4,6,12,14-hexaen-8-yl]acetamide

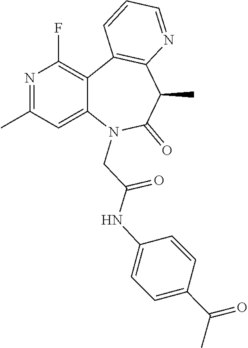 [SMILES: CC(=O)c1ccc(NC(=O)CN2C(=O)[C@H](C)c3ncccc3-c3c2cc(C)nc3F)cc1]

To a solution of intermediate L1 (20 mg, 0.055 mmol) and HATU (27 mg, 0.071 mmol) in dry DMF (0.275 mL) was added 1-(4-aminophenyl)ethanone (15 mg, 0.11 mmol) followed by diisopropylethylamine (47 μL, 0.285 mmol) and the reaction mixture was stirred at room temperature for 3 h. After complete conversion, the reaction mixture was purified by preparative HPLC (Purification Method P_B) to afford the title compound as a white solid (13.4 mg, yield: 56%). LC-MS m/z: [M+H]⁺: 433.2; purity: 95%. High Resolution Mass (Method HRMS_A2) m/z (+H); Observed Mass: 433.168; Expected Mass: 433.1676 (for C24H₂₁FN₄O₃); (rt: 1.87 min, purity: 96.3%).

### Example #2: N-[4-(cyclopropanecarbonyl)phenyl]-2-[(10R)-3-fluoro-5,10-dimethyl-9-oxo-4,8,12-triazatricyclo[9.4.0.0^{2,7}]pentadeca-1(11),2,4,6,12,14-hexaen-8-yl]acetamide

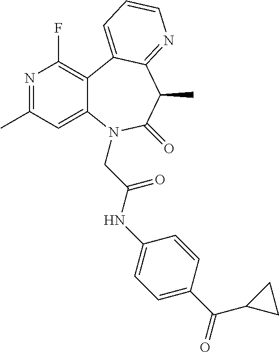 [SMILES: Cc1cc2c(c(F)n1)-c1cccnc1[C@@H](C)C(=O)N2CC(=O)Nc1ccc(C(=O)C2CC2)cc1]

The title compound was prepared according to the same procedure as for example #1 starting from intermediate L1 (30 mg, 0.08 mmol) and (4-aminophenyl)-cyclopropyl-methanone hydrochloride (32 mg, 0.16 mmol). After complete conversion, the reaction mixture was purified by preparative HPLC (Purification Method P_B) then by SFC (Phenomenex Luna 5 μm Silica (2) 100A, CO₂+EtOH 15%) to afford the title compound as a white solid (7.4 mg, yield: 19%). LC-MS (Method A2′) m/z [M+H]⁺: 459; rt: 4.24 min; purity: 97%. LC-MS (Method B2) m/z [M+H]⁺: 459; rt: 4.03 min; purity: 99%. ¹H NMR (400 MHz, DMSO-d₆) δ 10.58 (s, 1H), 8.67 (dd, J=4.7, 1.7 Hz, 1H), 8.13 (ddd, J=7.9, 4.7, 1.7 Hz, 1H), 8.03 (d, J=8.8 Hz, 2H), 7.72 (d, J=8.8 Hz, 2H), 7.50 (dd, J=7.9, 4.7 Hz, 1H), 7.38 (s, 1H), 4.68-4.53 (m, 2H), 3.79 (q, J=6.6 Hz, 1H), 2.85 (p, J=6.1 Hz, 1H), 1.49 (d, J=6.6 Hz, 3H), 1.04-0.97 (m, 4H). CH₃ protons under DMSO signal. High Resolution Mass (Method HRMS_A2) m/z (+H); Observed Mass: 459.183; Expected Mass: 459.1832 (for C₂₆H₂₃FN₄O₃); (rt: 2.06 min, purity: 100%).

### Example #3: N-(4-acetylphenyl)-2-[(10R)-3,14-difluoro-5,10-dimethyl-9-oxo-4,8,12-triazatricyclo[9.4.0.0^{2,7}]pentadeca-1(11),2,4,6,12,14-hexaen-8-yl]acetamide

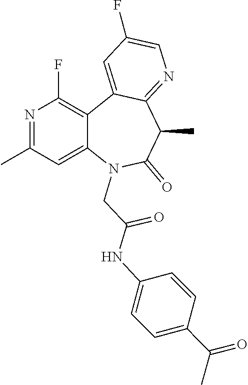 [SMILES: CC(=O)c1ccc(NC(=O)CN2C(=O)[C@H](C)c3ncc(F)cc3-c3c2cc(C)nc3F)cc1]

To a suspension of resolved Intermediate L2 (1.50 g, 5.45 mmol) in MeCN (22 mL) at room temperature was added intermediate C1 (1.15 g, 5.45 mmol). The resulting mixture was cooled down to 15° C. in an Easymax reactor under vigorous stirring before addition of K₂CO₃ (1.52 g, 10.9 mmol). The reaction mixture was stirred at 15° C. for 3 days. Water (40 mL) was added dropwise to the reaction mixture at room temperature. After stirring at room temperature for 30 min, the mixture was filtered over a glass frit. The solid was washed with water (3×20 mL) until pH=6. The solid was dried under high vacuum at 40° C. for 4 days and then purified by flash chromatography on silica gel (using a gradient of 0 to 100% EtOAc in Heptane as eluent). The product was triturated in Et₂O (15 mL), filtered over a glass frit, rinsed with Et₂O (5 mL) and dried under high vacuum at 40° C. for 16 h affording the title compound as a white solid (1.84 g, yield: 75%). LC-MS (Method A4) m/z [M+H]⁺: 451.3; rt: 3.88 min; purity: 99%. LC-MS (Method B4) m/z [M+H]⁺: 451.3; rt: 3.50 min; purity: 99%. ¹H NMR (400 MHz, DMSO-d₆) δ 10.56 (s, 1H), 8.70 (d, J=2.7 Hz, 1H), 8.14 (dt, J=9.6, 3.4 Hz, 1H), 7.93 (d, J=8.6 Hz, 2H), 7.69 (d, J=8.6 Hz, 2H), 7.39 (s, 1H), 4.61 (s, 2H), 3.81 (q, J=6.5 Hz, 1H), 2.52 (s, 3H), 1.49 (d, J=6.5 Hz, 3H). CH₃ protons under DMSO signal. ¹⁹F NMR (471 MHz, DMSO-d₆) δ −70.59 (d, J=3.4 Hz), −130.19 (d, J=9.6 Hz). Chiral purity: 100%; rt=3.07 min (first eluting enantiomer). For information, second eluting enantiomer rt=4.07 min. Both measured by HPLC (Reprosil Chiral NR-R from Dr Maisch, EtOH 50%−heptane 50%−DEA 0.1%). High Resolution Mass (Method HRMS_A2) m/z (+H); Observed Mass: 451.1586; Expected Mass: 451.1582 (for C₂₄H₂₀F₂N₄O₃); (rt: 2.05 min, purity: 100%).

### Example #4: enantiomer (10R) or (10S) of N-(4-acetylphenyl)-2-[14-fluoro-5,10-dimethyl-9-oxo-4,8,12-triazatricyclo[9.4.0.0^{2,7}]pentadeca-1(11),2,4,6,12,14-hexaen-8-yl]acetamide

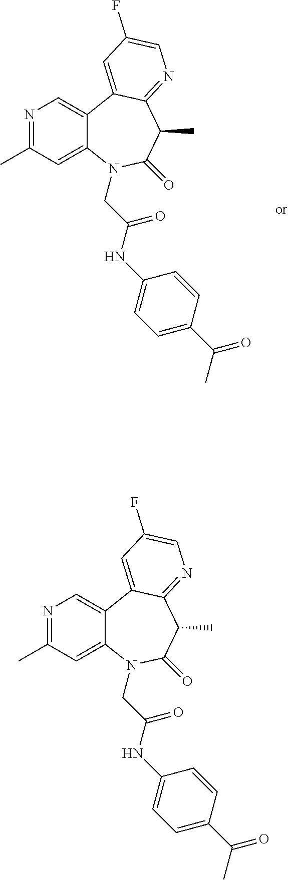 [SMILES: CC(=O)c1ccc(NC(=O)CN2C(=O)[C@H](C)c3ncc(F)cc3-c3cnc(C)cc32)cc1 ; CC(=O)c1ccc(NC(=O)CN2C(=O)[C@@H](C)c3ncc(F)cc3-c3cnc(C)cc32)cc1]

The title compound was prepared according to a similar procedure as for example #3 starting from intermediate L3 (600 mg, 2.33 mmol) and intermediate C1 (543 mg, 2.57 mmol) without using potassium iodide. After completion, the reaction mixture was poured into water. The resulting precipitate was filtered off and washed with water (3×20 mL). The solid was triturated in water, filtered off and washed with water (3×20 mL). The corresponding racemate was separated by Chiral SFC (Chiralpak IA from Daicel, CO₂+MeOH 20%). The second eluting enantiomer was then purified by column chromatography on silica gel (using a gradient of 0-100% EtOAc in Heptane then 0-15% MeOH in EtOAc as eluent) to afford the title compound as a white solid (193 mg, yield: 19%). LC-MS (Method A2′) m/z: [M+H]⁺: 433.1; rt: 3.34 min; purity: 100%. LC-MS (Method B2) m/z: [M+H]⁺: 433.1; rt: 3.79 min; purity: 100%. ¹H NMR (500 MHz, DMSO-d₆) δ 10.60 (s, 1H), 8.77 (s, 1H), 8.70 (d, J=2.8 Hz, 1H), 8.21 (dd, J=9.5, 2.8 Hz, 1H), 7.99-7.88 (m, 2H), 7.74-7.68 (m, 2H), 7.41 (s, 1H), 4.70-4.53 (m, 2H), 3.67 (q, J=6.6 Hz, 1H), 2.58 (s, 3H), 2.54 (s, 3H), 1.50 (d, J=6.6 Hz, 3H). ¹⁹F NMR (471 MHz, DMSO-d₆) δ −129.71 (d, J=9.5 Hz). Chiral purity: 100%; rt=3.54 min (second eluting enantiomer). For information, first eluting enantiomer rt=1.87 min. Both measured by HPLC (Chiralpak IA from Daicel, EtOH 50%−heptane 50%−DEA 0.1%). High Resolution Mass (Method HRMS_A2) m/z (+H); Observed Mass: 433.1679; Expected Mass: 433.1676 (for C₂₄H₂₁FN₄O₃); (rt: 1.58 min, purity: 100%).

### Example #5: N-(4-acetylphenyl)-2-[(10R)-3-chloro-14-fluoro-5,10-dimethyl-9-oxo-4,8,12-triazatricyclo[9.4.0.0^{2,7}]pentadeca-1(11),2,4,6,12,14-hexaen-8-yl]acetamide

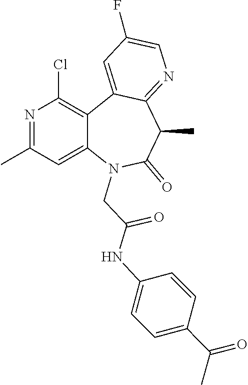 [SMILES: CC(=O)c1ccc(NC(=O)CN2C(=O)[C@H](C)c3ncc(F)cc3-c3c2cc(C)nc3Cl)cc1]

Example #5 was prepared according to a similar procedure as for example #3 and starting from intermediate L4 (600 mg, 2.06 mmol) and intermediate C1 (435 mg, 1 equiv). 50 mg of the crude was purified by reverse phase chromatography (basic elution) to give a white solid (30 mg)._LC-MS (Method A8) m/z [M+H]⁺: 467.3; rt: 4.12 min; purity: 98%. LC-MS (Method B8) m/z [M+H]⁺: 467.3; rt: 3.69 min; purity: 98%. ¹H NMR (400 MHz, DMSO-d₆) δ 10.53 (s, 1H), 8.69 (d, J=2.8 Hz, 1H), 8.23 (dd, J=9.7, 2.8 Hz, 1H), 7.92 (m, J=8.9 Hz, 2H), 7.68 (d, J=8.9 Hz, 2H), 7.47 (s, 1H), 4.61 (d, J=16.8 Hz, 1H), 4.54 (d, J=16.8 Hz, 1H), 3.83 (q, J=6.6 Hz, 1H), 2.54 (s, 3H), 2.52 (s, 3H), 1.48 (d, J=6.6 Hz, 3H). High Resolution Mass (Method HRMS_A2) m/z (+H); Observed Mass: 467.1288; Expected Mass: 467.1286 (for C₂₄H₂₀ClFN₄O₃); (rt: 2.09 min, purity: 100%).

### Example #6: enantiomer (10R) or (10S) of N-(4-acetylphenyl)-2-[3-chloro-5,10-dimethyl-9-oxo-4,8,12-triazatricyclo[9.4.0.0^{2,7}]pentadeca-1(11),2,4,6,12,14-hexaen-8-yl]acetamide

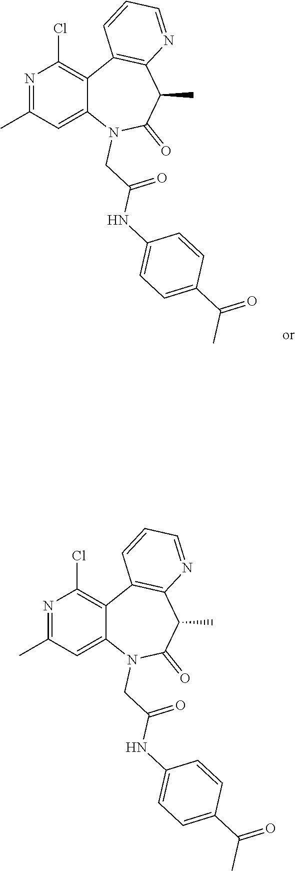 [SMILES: CC(=O)c1ccc(NC(=O)CN2C(=O)[C@H](C)c3ncccc3-c3c2cc(C)nc3Cl)cc1 ; CC(=O)c1ccc(NC(=O)CN2C(=O)[C@@H](C)c3ncccc3-c3c2cc(C)nc3Cl)cc1]

Example #6 was prepared according to a similar procedure as for example #3 and starting from intermediate L5 (252 mg, 0.92 mmol) and intermediate C1 (195 mg, 1 equiv). It was purified by trituration in isopropanol (1 mL) and diisopropylether (6 mL) to give a white solid (321 mg, yield: 76%). LC-MS (Method A2) m/z [M+H]⁺: 449.0; rt: 3.88 min; purity >99%. LC-MS (Method B2) m/z [M+H]⁺: 449.1; rt: 3.67 min; purity >99%. ¹H NMR (400 MHz, DMSO-d₆) δ 10.53 (s, 1H), 8.66 (dd, J=4.8, 1.6 Hz, 1H), 8.23 (dd, J=7.9, 1.7 Hz, 1H), 7.93 (d, J=8.5 Hz, 2H), 7.68 (d, J=8.5 Hz, 2H), 7.48 (dd, J=7.9, 4.8 Hz, 1H), 7.45 (s, 1H), 4.60 (d, J=16.8 Hz, 1H), 4.54 (d, J=16.8 Hz, 1H), 3.81 (q, J=6.6 Hz, 1H), 2.53 (s, 3H), 2.52 (s, 3H), 1.49 (d, J=6.6 Hz, 3H). High Resolution Mass (Method HRMS_A2) m/z (+H); Observed Mass: 449.1386; Expected Mass: 449.138 (for C₂₄H₂₁ClN₄O₃); (rt: 1.9 min, purity: 100%).

### Example #7: enantiomer (10R) or (10S) of N-(4-acetylphenyl)-2-[5-(3,3-difluoroazetidin-1-yl)-3-fluoro-10-methyl-9-oxo-4,8,12-triazatricyclo[9.4.0.0^{2,7}]pentadeca-1(11),2,4,6,12,14-hexaen-8-yl]acetamide

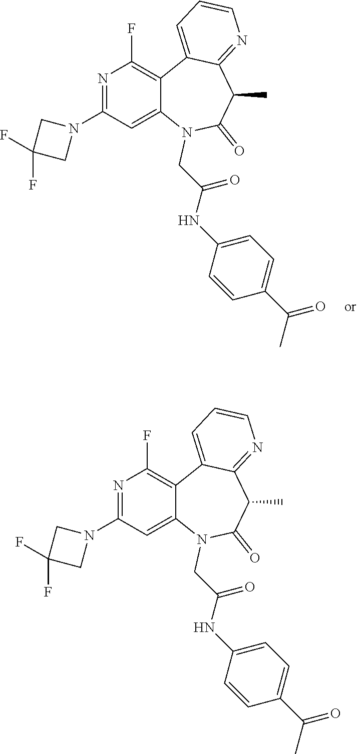 [SMILES: CC(=O)c1ccc(NC(=O)CN2C(=O)[C@H](C)c3ncccc3-c3c2cc(N2CC(F)(F)C2)nc3F)cc1 ; CC(=O)c1ccc(NC(=O)CN2C(=O)[C@@H](C)c3ncccc3-c3c2cc(N2CC(F)(F)C2)nc3F)cc1]

To a solution of intermediate L6 (300 mg, 0.63 mmol) and 3,3-difluoroazetidine hydrochloride (190 mg, 1.40 mmol) in acetonitrile (6 mL) was added N,N-diisopropylethylamine (0.42 mL, 2.5 mmol) at room temperature and the mixture was stirred overnight. The mixture was stirred for another 5 days at room temperature and N,N-diisopropylethylamine (0.42 mL) was again added three times after daily analysis. The reaction mixture was evaporated then diluted with dichloromethane (10 mL) and washed with water (20 mL). The organic layer was dried over MgSO₄, filtered and concentrated under vacuo to afford 360 mg of a crude solid which was purified by SFC (GreenSep-Nitro column, 80 mL/min, CO₂₊₃₅% MeOH) to afford racemic N-(4-acetylphenyl)-2-[5-(3,3-difluoroazetidin-1-yl)-3-fluoro-10-methyl-9-oxo-4,8,12-triazatricyclo[9.4.0.0^{2,7}]pentadeca-1(11),2,4,6,12,14-hexaen-8-yl]acetamide (171 mg, major regioisomer, second eluting isomer) as well as the minor regioisomer (45 mg, first eluting isomer). The racemate was separated by Chiral HPLC (Reprosil NR-R from Dr Maisch, EtOH 50%−heptane 50%, 30 mL/min) to give the title product as a white solid (78 mg, yield: 24%). LC-MS (Method B2) m/z [M+H]⁺: 510.0; rt: 4.53 min; purity: 99%. LC-MS (Method A2) m/z [M+H]⁺: 510.0; rt: 4.40 min; purity >99%. Chiral purity >99%; rt: 3.39 min (first eluting enantiomer, second eluting enantiomer rt: 5.22 min), measured by UHPLC (Reprosil Chiral NR-R from Dr Maisch, EtOH 50%−heptane 50%−DEA 0.1%). High Resolution Mass (Method HRMS_A2) m/z (+H); Observed Mass: 510.1765; Expected Mass: 510.1753 (for C₂₆H₂₂F₃N₅O₃); (rt: 2.15 min, purity: 100%).

### Example #8: enantiomer (10R) or (10S) of N-(4-acetylphenyl)-2-[3-fluoro-5-(3-fluoroazetidin-1-yl)-10-methyl-9-oxo-4,8,12-triazatricyclo[9.4.0.0^{2,7}]pentadeca-1(11),2,4,6,12,14-hexaen-8-yl]acetamide

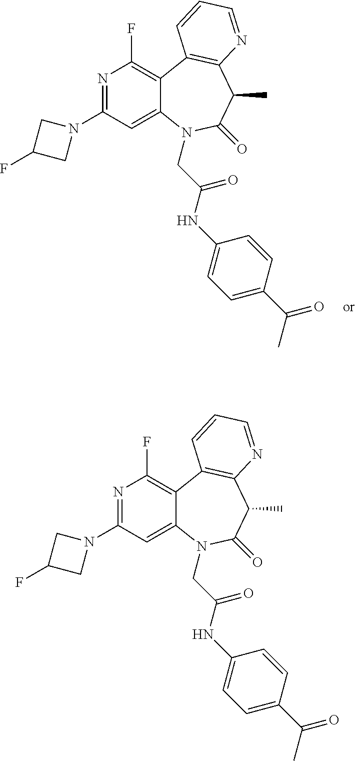 [SMILES: CC(=O)c1ccc(NC(=O)CN2C(=O)[C@H](C)c3ncccc3-c3c2cc(N2CC(F)C2)nc3F)cc1 ; CC(=O)c1ccc(NC(=O)CN2C(=O)[C@@H](C)c3ncccc3-c3c2cc(N2CC(F)C2)nc3F)cc1]

Example #8 was prepared according to a similar procedure as for example #7 starting from intermediate L6 (310 mg, 0.65 mmol) and 3-fluoroazetidine hydrochloride (98 mg, 0.83 mmol). The crude mixture (374 mg) was purified by SFC (2-EP column from Kromasil, 80 mL/min, CO₂+5 to 50% MeOH gradient) to afford racemic N-(4-acetylphenyl)-2-[3-fluoro-5-(3-fluoroazetidin-1-yl)-10-methyl-9-oxo-4,8,12-triazatricyclo[9.4.0.02,7]pentadeca-1(11),2,4,6,12,14-hexaen-8-yl]acetamide (110 mg, major regioisomer, second eluting isomer) as well as the minor regioisomer (35 mg, first eluting isomer). The racemate was separated by Chiral HPLC ((R,R)Whelk-O 1 from Regis Technologies, EtOH 100%) to give the title product as a white solid (54 mg, yield: 17%). LC-MS (Method B2) m/z [M+H]⁺: 492.0; rt: 4.42 min; purity: 99%. LC-MS (Method A2) m/z [M+H]⁺: 492.0; rt: 4.17 min; purity: 99%. Chiral purity >99%; rt: 2.46 min (first eluting enantiomer, second eluting enantiomer rt: 2.92 min), measured by UHPLC (column (R,R)Whelk-01, EtOH 100%−DEA 0.1%). High Resolution Mass (Method HRMS_A2) m/z (+H); Observed Mass: 492.1859; Expected Mass: 492.1847 (for C₂₆H₂₃F₂N₅O₃); (rt: 2.01 min, purity: 98.5%).

### Example #9: N-(4-acetylphenyl)-2-[3-chloro-5-(2,2-difluoro-5-azaspiro[2.3]hexan-5-yl)-9-oxo-4,8,12-triazatricyclo[9.4.0.0^{2,7}]pentadeca-1(11),2,4,6,12,14-hexaen-8-yl]acetamide

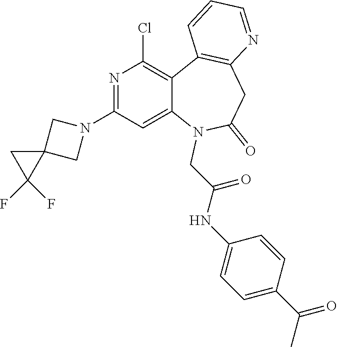 [SMILES: CC(=O)c1ccc(NC(=O)CN2C(=O)Cc3ncccc3-c3c2cc(N2CC4(C2)CC4(F)F)nc3Cl)cc1]

The title compound was prepared according to a similar procedure as for example #3 starting from Intermediate L7 (8.0 mg, 0.019 mmol) and Intermediate C1 (5.4 mg, 0.024 mmol). After completion, the reaction mixture was poured into water. The resulting precipitate was filtered off and washed with water. The residue was purified by preparative HPLC (Purification Method P_B) to afford the title compound as a white solid (2.4 mg, yield: 24%). LC-MS (Method A4) m/z: [M+H]⁺: 538.2; rt: 4.25 min; purity: 99%. LC-MS (Method B4) m/z: [M+H]⁺: 538.2; rt: 3.89 min; purity: 99%. ¹H NMR (400 MHz, DMSO-d₆) δ 10.52 (s, 1H), 8.53 (dd, J=4.8, 1.7 Hz, 1H), 8.15 (dd, J=7.9, 1.7 Hz, 1H), 7.95-7.88 (m, 2H), 7.69-7.63 (m, 2H), 7.46 (dd, J=7.9, 4.8 Hz, 1H), 6.56 (s, 1H), 4.62 (d, J=16.7 Hz, 1H), 4.50 (d, J=16.7 Hz, 1H), 4.27-4.11 (m, 4H), 3.90 (d, J=12.5 Hz, 1H), 3.62 (d, J=12.5 Hz, 1H), 2.52 (s, 3H), 1.86-1.73 (m, 2H). ¹⁹F NMR (376 MHz, DMSO-d₆) δ −137.56 (t, J=8.7 Hz). High Resolution Mass (Method HRMS_A2) m/z (+H); Observed Mass: 538.1469; Expected Mass: 538.1458 (for C₂₇H₂₂ClF₂N₅O₃); (rt: 2.14 min, purity: 100%).

### Example #10: N-(4-acetylphenyl)-2-(1-fluoro-3-methyl-6-oxo-7H-pyrido[4,3-d][3]benzazepin-5-yl)acetamide

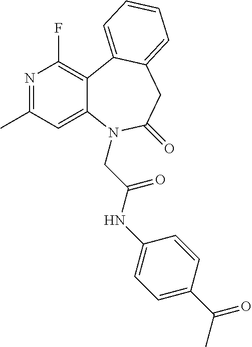 [SMILES: CC(=O)c1ccc(NC(=O)CN2C(=O)Cc3ccccc3-c3c2cc(C)nc3F)cc1]

The title compound was prepared according to the same procedure as for example #1 starting from Intermediate L8 (15 mg, 0.04 mmol) and 1-(4-aminophenyl)ethanone (12.2 mg, 0.09 mmol). After complete conversion, the reaction mixture was purified by preparative HPLC (Purification Method P_B) to afford the title compound (10.2 mg, yield: 55%). LC-MS m/z: [M+H]⁺: 418.2; purity: 96%. High Resolution Mass (Method HRMS_A2) m/z (+H); Observed Mass: 418.1558; Expected Mass: 418.1567 (for C₂₄H₂₀FN₃O₃); (rt: 2.02 min, purity: 94.54%).

### Example #11: enantiomer (10R) or (10S) of N-(4-acetylphenyl)-2-[3-fluoro-5-methoxy-10-methyl-9-oxo-4,8,12-triazatricyclo[9.4.0.0^{2,7}]pentadeca-1(11),2(7),3,5,12,14-hexaen-8-yl]acetamide

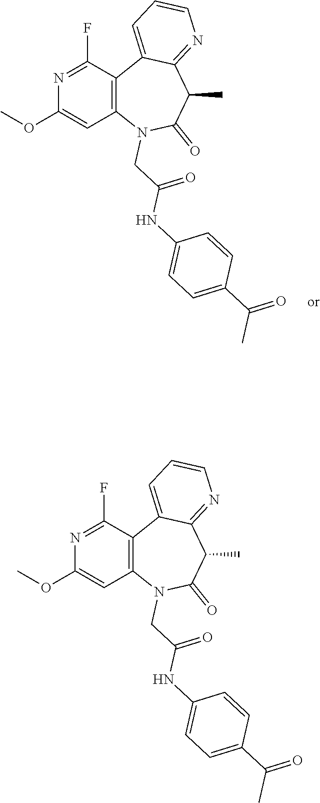 [SMILES: COc1cc2c(c(F)n1)-c1cccnc1[C@@H](C)C(=O)N2CC(=O)Nc1ccc(C(C)=O)cc1 ; COc1cc2c(c(F)n1)-c1cccnc1[C@H](C)C(=O)N2CC(=O)Nc1ccc(C(C)=O)cc1]

To a solution of Intermediate L9 (30 mg, 0.07 mmol) in MeCN (0.73 mL) were added at room temperature 1-(4-aminophenyl)ethanone (20 mg, 0.15 mmol), TCFH (42 mg, 0.15 mmol) and NMI (23 μL, 0.29 mmol). The resulting mixture was stirred at room temperature for 3 h. After completion, water was added and the reaction mixture was extracted with DCM (3 x, separation over a phase separator). The combined organic layers were concentrated under vacuum. The residue was purified by preparative HPLC (Purification Method P_B) to afford the title compound as a white solid (26.4 mg, yield: 79%). LC-MS (Method A4) m/z: [M+H]⁺: 449.4; rt: 3.86 min; purity: 97%. LC-MS (Method B4) m/z: [M+H]⁺: 449.3; rt: 3.50 min; purity: 97%; chiral purity >99%. ¹H NMR (400 MHz, DMSO-d₆) δ 10.56 (s, 1H), 8.64 (dd, J=4.8, 1.6 Hz, 1H), 8.13-8.05 (m, 1H), 7.96-7.89 (m, 2H), 7.71-7.64 (m, 2H), 7.48 (dd, J=7.9, 4.8 Hz, 1H), 6.90 (s, 1H), 4.61 (s, 2H), 3.92 (s, 3H), 3.85 (q, J=6.6 Hz, 1H), 2.52 (s, 3H), 1.49 (d, J=6.6 Hz, 3H). ¹⁹F NMR (376 MHz, DMSO-d₆) δ −72.04 (d, J=4.6 Hz). High Resolution Mass (Method HRMS_A2) m/z (+H); Observed Mass: 449.1635; Expected Mass: 449.1625 (for C₂₄H₂₁FN₄O₄); (rt: 2.04 min, purity: 98.43%).

### Example #12: enantiomer (10R) or (10S) of N-(4-acetylphenyl)-2-[14-methoxy-5,10-dimethyl-9-oxo-4,8,12-triazatricyclo[9.4.0.0^{2,7}]pentadeca-1(11),2,4,6,12,14-hexaen-8-yl]acetamide

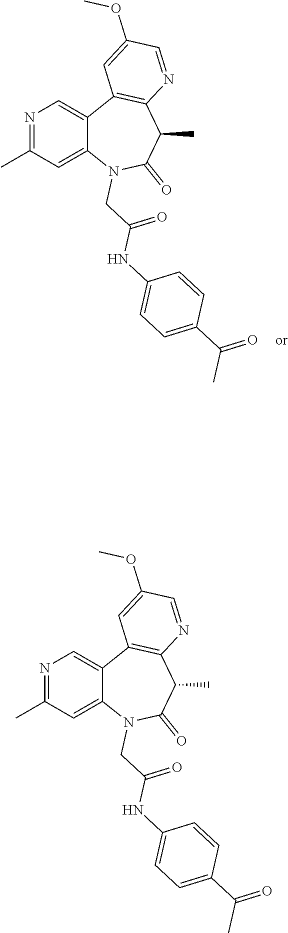 [SMILES: COc1cnc2c(c1)-c1cnc(C)cc1N(CC(=O)Nc1ccc(C(C)=O)cc1)C(=O)[C@@H]2C ; COc1cnc2c(c1)-c1cnc(C)cc1N(CC(=O)Nc1ccc(C(C)=O)cc1)C(=O)[C@H]2C]

Example #12 was prepared according to a similar procedure as for example #3 starting from intermediate L11 (90 mg, 0.33 mmol) and intermediate C1 (71 mg, 1 equiv). It was purified by reverse phase chromatography (Waters XBridge OBD MS C18 column (5 μm, 30×50 mm). Gradient elution is performed with solvent A (H₂O 95%−ACN 5%+NH₄HCO₃ 50 mM+200 μL/L NH₄OH) and solvent B (100% ACN) (pH~8.5), flow rate: 35 to 45 mL/min) and the enantiomers were separated by Chiral HPLC (Chiralpak IB from Daicel, EtOH 100%) to give the title product as a white solid (28 mg, yield: 19%). LC-MS (Method B2) m/z [M+H]⁺: 445.0; rt: 3.76 min; purity: 95%. LC-MS (Method A2) m/z [M+H]⁺: 445.0; rt: 3.37 min; purity: 96%. Chiral purity: 99%; rt: 2.52 min (second eluting enantiomer, first eluting enantiomer rt: 1.86 min), measured by HPLC (Chiralpak IB, EtOH 100%−DEA 0.1%). ¹H NMR (400 MHz, DMSO-d₆) δ 10.56 (s, 1H), 8.79 (s, 1H), 8.39 (d, J=3.0 Hz, 1H), 7.93 (d, J=8.7 Hz, 2H), 7.76-7.67 (m, 3H), 7.37 (s, 1H), 4.64 (d, J=16.8 Hz, 1H), 4.53 (d, J=16.8 Hz, 1H), 3.93 (s, 3H), 3.54 (q, J=6.8 Hz, 1H), 2.56 (s, 3H), 2.53 (s, 3H), 1.47 (d, J=6.8 Hz, 3H). High Resolution Mass (Method HRMS_A2) m/z (+H); Observed Mass: 445.1882; Expected Mass: 445.1876 (for C₂₅H₂₄N₄O₄); (rt: 1.5 min, purity: 95.81%).

### Example #13: enantiomer (10R) or (10S) of N-(4-acetylphenyl)-2-[3-methoxy-5,10-dimethyl-9-oxo-4,8,12-triazatricyclo[9.4.0.0^{2,7}]pentadeca-1(15),2(7),3,5,11,13-hexaen-8-yl]acetamide

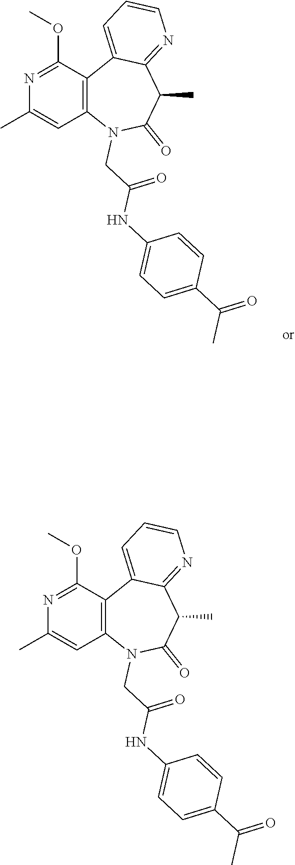 [SMILES: COc1nc(C)cc2c1-c1cccnc1[C@@H](C)C(=O)N2CC(=O)Nc1ccc(C(C)=O)cc1 ; COc1nc(C)cc2c1-c1cccnc1[C@H](C)C(=O)N2CC(=O)Nc1ccc(C(C)=O)cc1]

The title compound was prepared according to a similar procedure as for example #3 starting from Intermediate L12 (141 mg, 0.52 mmol) and Intermediate C1 (137 mg, 0.63 mmol). After purification by preparative HPLC (Purification Method P_B), the corresponding racemate was separated by Chiral HPLC (Reprosil NR-R from Dr Maisch, EtOH 50%−heptane 50%) to afford the title compound as a white solid (59.0 mg, yield: 25%). LC-MS (Method A2) m/z: [M+H]⁺: 445.1; rt: 4.06 min; purity: 99%. LC-MS (Method B2) m/z: [M+H]⁺: 445.1; rt: 4.24 min; purity: 99%. ¹H NMR (500 MHz, DMSO-d₆) δ 10.54 (s, 1H), 8.58 (dd, J=4.7, 1.7 Hz, 1H), 8.15 (dd, J=7.9, 1.7 Hz, 1H), 7.99-7.87 (m, 2H), 7.75-7.62 (m, 2H), 7.42 (dd, J=7.9, 4.7 Hz, 1H), 7.03 (s, 1H), 4.56-4.47 (m, 2H), 3.90 (s, 3H), 3.64 (q, J=6.7 Hz, 1H), 2.53 (s, 3H), 2.46 (s, 3H), 1.47 (d, J=6.7 Hz, 3H). Chiral purity: 100%; rt=3.15 min (first eluting enantiomer). For information, second eluting enantiomer rt=4.85 min. Both measured by HPLC (Reprosil NR-R from Dr Maisch, EtOH 50%−heptane 50%−DEA 0.1%). High Resolution Mass (Method HRMS_A2) m/z (+H); Observed Mass: 445.1879; Expected Mass: 445.1876 (for C₂₅H₂₄N₄O₄); (rt: 2.03 min, purity: 100%).

### Example #14: enantiomer (10R) or (10S) of N-(4-acetylphenyl)-2-(3-methoxy-10-methyl-9-oxo-4,8,12-triazatricyclo[9.4.0.0^{2,7}]pentadeca-1(11),2(7),3,5,12,14-hexaen-8-yl)acetamide

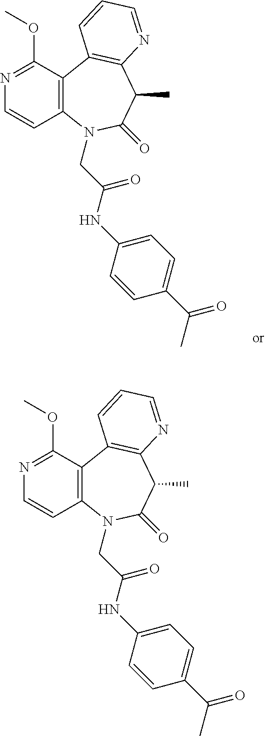 [SMILES: COc1nccc2c1-c1cccnc1[C@@H](C)C(=O)N2CC(=O)Nc1ccc(C(C)=O)cc1 ; COc1nccc2c1-c1cccnc1[C@H](C)C(=O)N2CC(=O)Nc1ccc(C(C)=O)cc1]

The title compound was prepared according to a similar procedure as for example #3 starting from intermediate L14 (140 mg, 0.50 mmol) and intermediate C1 (147 mg, 0.61 mmol). After purification by preparative HPLC (Purification Method P_B), the corresponding racemate was separated by Chiral HPLC (Whelk O-1 (R,R) from Regis Technology, EtOH 100%) to afford the title compound as a white solid (55.6 mg, yield: 25%). LC-MS (Method A2′) m/z: [M+H]⁺: 431.1; rt: 3.92 min; purity: 99%. LC-MS (Method B2) m/z: [M+H]⁺: 431.1; rt: 4.36 min; purity: 99%. ¹H NMR (400 MHz, DMSO-d₆) δ 10.56 (s, 1H), 8.61 (dd, J=4.7, 1.7 Hz, 1H), 8.23 (d, J=5.8 Hz, 1H), 8.19 (dd, J=7.9, 1.7 Hz, 1H), 7.93 (d, J=8.8 Hz, 2H), 7.68 (d, J=8.8 Hz, 2H), 7.44 (dd, J=7.9, 4.7 Hz, 1H), 7.16 (d, J=5.8 Hz, 1H), 4.63-4.46 (m, 2H), 3.92 (s, 3H), 3.66 (q, J=6.6 Hz, 1H), 2.52 (s, 3H), 1.49 (d, J=6.6 Hz, 3H). Chiral purity: 100%; rt=2.16 min (first eluting enantiomer). For information, second eluting enantiomer rt=2.54 min. Both measured by HPLC (Whelk O-1 (R,R) from Regis Technology, EtOH 100%−DEA 0.1%).

### Example #15: 2-[(10R)-3-fluoro-5,10-dimethyl-9-oxo-4,8,12-triazatricyclo[9.4.0.0^{2,7}]pentadeca-1(11),2,4,6,12,14-hexaen-8-yl]-N-(4-propanoylphenyl)acetamide

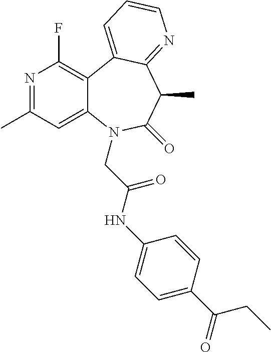 [SMILES: CCC(=O)c1ccc(NC(=O)CN2C(=O)[C@H](C)c3ncccc3-c3c2cc(C)nc3F)cc1]

The title compound was prepared according to a similar procedure as for example #1 starting from Intermediate L1 (53 mg, 0.11 mmol) and 4′-aminopropiophenone (15 mg, 0.10 mmol). The crude mixture was purified by preparative HPLC (Purification Method P_B) to afford the title compound as a white solid (21 mg, yield: 47%). LC-MS (Method A4) m/z: [M+H]⁺: 447.2; rt: 4.10 min; purity: 96%. LC-MS (Method B4) m/z: [M+H]⁺: 447.3; rt: 3.53 min; purity: 96%. ¹H NMR (400 MHz, DMSO-d₆) δ 10.57 (s, 1H), 8.67 (dd, J=4.8, 1.6 Hz, 1H), 8.17-8.09 (m, 1H), 7.94 (d, J=8.5 Hz, 2H), 7.69 (d, J=8.5 Hz, 2H), 7.50 (dd, J=7.9, 4.8 Hz, 1H), 7.38 (s, 1H), 4.60 (s, 2H), 3.79 (q, J=6.6 Hz, 1H), 2.99 (q, J=7.2 Hz, 2H), 1.49 (d, J=6.6 Hz, 3H), 1.07 (t, J=7.2 Hz, 3H). CH₃ protons under DMSO signal. ¹⁹F NMR (376 MHz, DMSO-d₆) δ −70.96 (d, J=5.1 Hz).

### Example #16: enantiomer (10R) or (10S) of N-(4-acetyl-3-hydroxy-phenyl)-2-(3-fluoro-5,10-dimethyl-9-oxo-4,8,12-triazatricyclo[9.4.0.0^{2,7}]pentadeca-1(11),2(7),3,5,12,14-hexaen-8-yl)acetamide

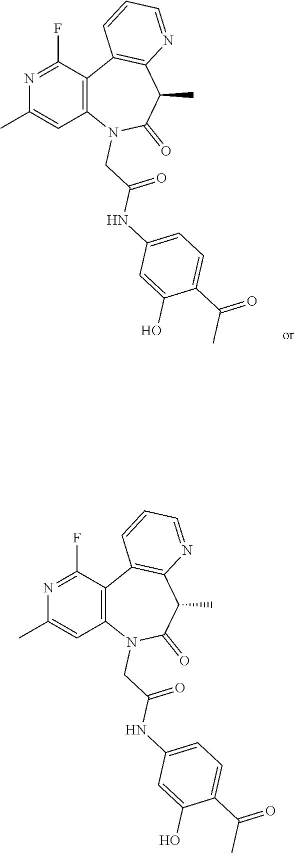 [SMILES: CC(=O)c1ccc(NC(=O)CN2C(=O)[C@H](C)c3ncccc3-c3c2cc(C)nc3F)cc1O ; CC(=O)c1ccc(NC(=O)CN2C(=O)[C@@H](C)c3ncccc3-c3c2cc(C)nc3F)cc1O]

The title compound was prepared according to a similar procedure as for example #3 starting from Intermediate L1_6 (50 mg, 0.19 mmol) and Intermediate C2 (49 mg, 0.21 mmol). After purification by preparative HPLC (Purification Method P_B), the corresponding racemate was separated by Chiral HPLC (Reprosil NR-R from Dr Maisch, EtOH 100%) to afford the title compound as a white solid (9.9 mg, yield: 11%). LC-MS (Method A2) m/z: [M+H]⁺: 449.2; rt: 4.05 min; purity: 99%. LC-MS (Method B2) m/z: [M+H]⁺: 449.1; rt: 3.88 min; purity: 99%. ¹H NMR (500 MHz, DMSO-d₆) δ 12.29 (s, 1H), 10.59 (s, 1H), 8.67 (dd, J=4.7, 1.8 Hz, 1H), 8.13 (ddd, J=7.9, 4.7, 1.8 Hz, 1H), 7.86 (d, J=8.7 Hz, 1H), 7.50 (dd, J=7.9, 4.7 Hz, 1H), 7.36 (s, 1H), 7.32 (d, J=2.1 Hz, 1H), 7.05 (dd, J=8.7, 2.1 Hz, 1H), 4.58 (s, 2H), 3.79 (q, J=6.6 Hz, 1H), 2.57 (s, 3H), 1.49 (d, J=6.6 Hz, 3H). CH₃ protons under DMSO signal. ¹⁹F NMR (471 MHz, DMSO-d₆) δ −70.97 (d, J=4.7 Hz). Chiral purity: 100%; rt=2.23 min (first eluting enantiomer). For information, second eluting enantiomer rt=2.77 min. Both measured by HPLC (Reprosil NR-R from Dr Maisch, EtOH 100%−DEA 0.1%).

### Example #17: enantiomer (10R) or (10S) of N-(4-acetylphenyl)-2-(3,14-difluoro-5-methoxy-10-methyl-9-oxo-4,8,12-triazatricyclo[9.4.0.0^{2,7}]pentadeca-1(11),2(7),3,5,12,14-hexaen-8-yl)acetamide

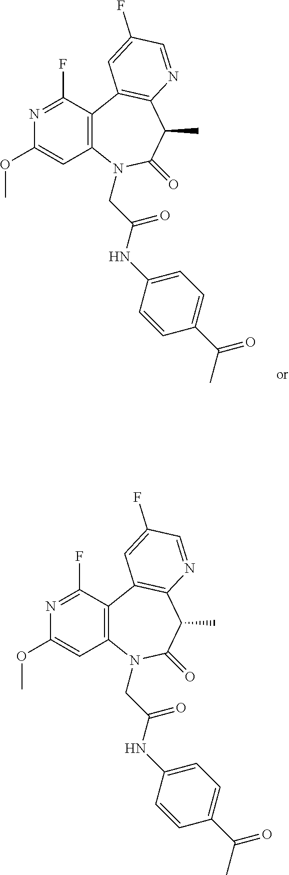 [SMILES: COc1cc2c(c(F)n1)-c1cc(F)cnc1[C@@H](C)C(=O)N2CC(=O)Nc1ccc(C(C)=O)cc1 ; COc1cc2c(c(F)n1)-c1cc(F)cnc1[C@H](C)C(=O)N2CC(=O)Nc1ccc(C(C)=O)cc1]

The title compound was prepared according to a similar procedure as for example #3 starting from Intermediate L15 (333 mg, 0.64 mmol) and Intermediate C1 (187 mg, 0.77 mmol). After purification by reverse phase chromatography (basic elution), the corresponding racemate was separated by Chiral SFC (Chiralpak IB from Daicel, CO₂+EtOH 20%) to afford the title compound as a white solid (94 mg, yield: 32%). LC-MS (Method A2′) m/z: [M+H]⁺: 467.1; rt: 4.51 min; purity: 99%. LC-MS (Method B2) m/z: [M+H]⁺: 467.1; rt: 4.36 min; purity: 98%. ¹H NMR (500 MHz, DMSO-d₆) δ 10.59 (s, 1H), 8.67 (d, J=2.9 Hz, 1H), 8.11 (dt, J=9.5, 3.4 Hz, 1H), 7.93 (d, J=8.8 Hz, 2H), 7.68 (d, J=8.8 Hz, 2H), 6.92 (s, 1H), 4.62 (s, 2H), 3.92 (s, 3H), 3.87 (q, J=6.6 Hz, 1H), 2.52 (s, 3H), 1.47 (d, J=6.6 Hz, 3H). ¹⁹F NMR (471 MHz, DMSO-d₆) δ −71.56 (t, J=3.4 Hz), −130.18 (d, J=9.5 Hz). Chiral purity: 100%; rt=2.42 min (second eluting enantiomer). For information, first eluting enantiomer rt=1.94 min. Both measured by HPLC (Chiralpak IB from Daicel, MeOH 100%−DEA 0.1%).

### Example #18: enantiomer (10R) or (10S) of 2-[3,14-difluoro-5,10-dimethyl-9-oxo-4,8,12-triazatricyclo[9.4.0.0^{2,7}]pentadeca-1(11),2(7),3,5,12,14-hexaen-8-yl]-N-(3-oxobenzofuran-6-yl)acetamide

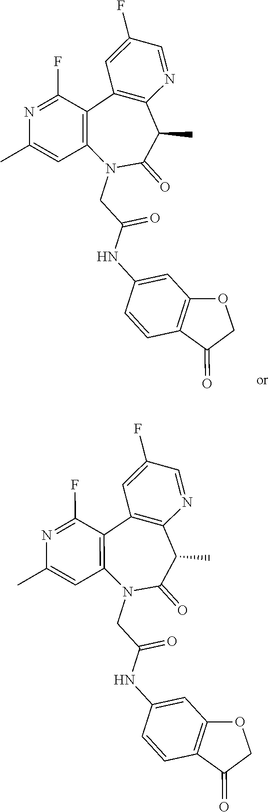 [SMILES: Cc1cc2c(c(F)n1)-c1cc(F)cnc1[C@@H](C)C(=O)N2CC(=O)Nc1ccc2c(c1)OCC2=O ; Cc1cc2c(c(F)n1)-c1cc(F)cnc1[C@H](C)C(=O)N2CC(=O)Nc1ccc2c(c1)OCC2=O]

To a mixture of Intermediate L16 (297 mg, 0.89 mmol) and Intermediate L17 (394 mg, 1.34 mmol) in dry 1,4-dioxane (9 mL) was added, at room temperature, cesium carbonate (882 mg, 2.68 mmol). The resulting mixture was flushed with N₂ before addition of BrettPhos Pd G3 (81 mg, 0.09 mmol) and BrettPhos (48 mg, 0.09 mmol). The reaction mixture was then stirred at 110° C. for 2 h. After completion, the reaction was diluted with EtOAc (10 mL), filtered over a pad of celite and rinsed with EtOAc. The filtrate was concentrated undervacuum. After purification by flash chromatography on silica gel (using a gradient of 0 to 100% EtOAc in Heptane as eluent), the corresponding racemate was separated by Chiral HPLC (Chiralpak IB from Daicel, MeOH 100%) to afford the title compound as a yellow solid (17.9 mg, yield: 4%). LC-MS (Method A2′) m/z: [M+H]⁺: 465.1; rt: 4.15 min; purity: 96%. LC-MS (Method B2) m/z: [M+H]⁺: 465.0; rt: 3.94 min; purity: 99%. ¹H NMR (400 MHz, DMSO-d₆) δ 10.77 (s, 1H), 8.71 (d, J=2.8 Hz, 1H), 8.15 (dt, J=9.7, 3.6 Hz, 1H), 7.63 (d, J=1.7 Hz, 1H), 7.59 (d, J=8.4 Hz, 1H), 7.38 (s, 1H), 7.17 (dd, J=8.4, 1.7 Hz, 1H), 4.77 (s, 2H), 4.62 (s, 2H), 3.82 (q, J=6.5 Hz, 1H), 1.48 (d, J=6.5 Hz, 3H). CH₃ protons under DMSO signal. ¹⁹F NMR (471 MHz, DMSO) δ −70.60 (d, J=3.6 Hz), −130.15 (d, J=9.7 Hz). Chiral purity: 98%; rt=2.41 min (second eluting enantiomer). For information, first eluting enantiomer rt=1.91 min. Both measured by HPLC (Chiralpak IB from Daicel, EtOH 100%−DEA 0.1%).

### Example #19: enantiomer (10R) or (10S) of N-(4-acetylphenyl)-2-(10-cyano-2-fluoro-5,9-dimethyl-6-oxo-5H-pyrido[2,3-d][1]benzazepin-7-yl)acetamide

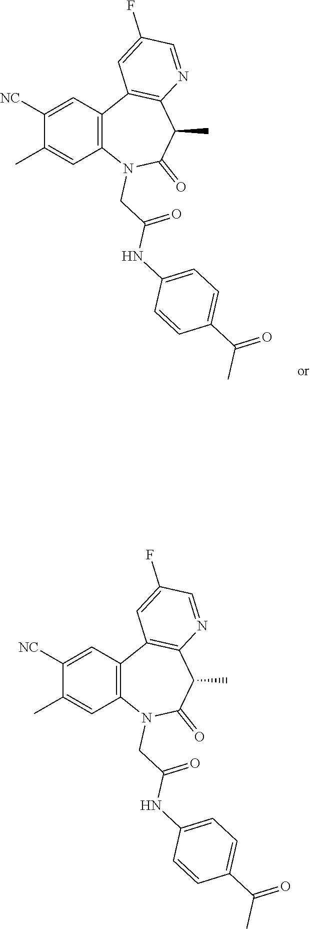 [SMILES: CC(=O)c1ccc(NC(=O)CN2C(=O)[C@H](C)c3ncc(F)cc3-c3cc(C#N)c(C)cc32)cc1 ; CC(=O)c1ccc(NC(=O)CN2C(=O)[C@@H](C)c3ncc(F)cc3-c3cc(C#N)c(C)cc32)cc1]

To a solution of Intermediate L22 (15 mg, 0.05 mmol) and Intermediate C1 (13 mg, 0.05 mmol) in N,N-dimethylformamide (1 mL) was added potassium carbonate (15 mg, 0.11 mmol). The reaction mixture was stirred at room temperature for 20 h. After completion, water and ethyl acetate were added and the reaction mixture was extracted three times with ethyl acetate. The combined organic layers were dried over MgSO₄, filtered, and concentrated under vacuum. The residue was purified by preparative HPLC (Purification Method P_B) to afford the corresponding racemate compound as a white solid (12 mg, yield: 48%). LC-MS (Method B5) m/z: [M+H]⁺: 457; rt: 3.37 min; purity: 97.8%. LC-MS (Method A8) m/z: [M+H]⁺: 457; rt: 3.75 min; purity: 99.4%. The racemate was purified by Chiral SFC (column Lux A1 from Phenomenex, CO₂+MeOH:MeCN (1:1) 60%) to give the title product. Chiral purity >95%; rt: 3.26 min (second eluting enantiomer). For information, first eluting enantiomer rt: 1.55 min). Both measured by SFC (column Lux A1 from Phenomenex, CO₂+MeOH 50%+NH₃ 0.1%).

### Example #20: enantiomer (10R) or (10S) of N-(4-acetylphenyl)-2-[(10)-3-fluoro-14-methoxy-5,10-dimethyl-9-oxo-4,8,12-triazatricyclo[9.4.0.0^{2,7}]pentadeca-1(11),2(7),3,5,12,14-hexaen-8-yl]acetamide

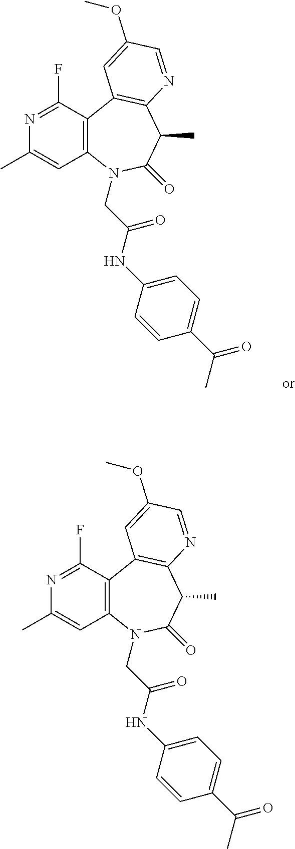 [SMILES: COc1cnc2c(c1)-c1c(cc(C)nc1F)N(CC(=O)Nc1ccc(C(C)=O)cc1)C(=O)[C@@H]2C ; COc1cnc2c(c1)-c1c(cc(C)nc1F)N(CC(=O)Nc1ccc(C(C)=O)cc1)C(=O)[C@H]2C]

To a solution of Intermediate L10 (100 mg, 0.35 mmol) in N,N-dimethylformamide (1.7 mL) were added at room temperature potassium carbonate (146 mg, 1.05 mmol) and Intermediate C1 (74 mg, 0.35 mmol). The resulting mixture was stirred at room temperature for 1 h. After completion, water and ethyl acetate was added and the reaction mixture were extracted three times with ethyl acetate. The combined organic layers were dried over MgSO₄, filtered, and concentrated under vacuum. The residue was purified by preparative HPLC (Purification Method P_B) to afford the corresponding racemate as a white solid (75.0 mg, yield: 47%). The racemate was separated by Chiral HPLC (Reprosil Chiral NR-R from Dr Maisch, EtOH 50%−heptane 50%+DEA 0.1%) to give the title product (24.5 mg, yield: 15%). Chiral purity >99%; rt: 3.97 min (first eluting enantiomer). For information, second eluting enantiomer rt: 5.43 min). Both measured by HPLC (Reprosil Chiral NR-R from Dr Maisch, EtOH 50%−heptane 50%+DEA 0.1%). LC-MS (Method A2′) m/z: [M+H]⁺: 463.0; rt: 4.14 min; purity: 99%. LC-MS (Method B2) m/z: [M+H]⁺: 463.0; rt: 4.24 min; purity: 98%. ¹H NMR (400 MHz, DMSO-d₆) δ 10.55 (s, 1H), 8.40 (d, J=2.8 Hz, 1H), 7.96-7.89 (m, 2H), 7.73-7.66 (m, 3H), 7.36 (s, 1H), 4.61 (d, J=16.9 Hz, 1H), 4.54 (d, J=16.9 Hz, 1H), 3.88 (s, 3H), 3.68 (q, J=6.6 Hz, 1H), 2.52 (s, 3H), 2.50 (s, 3H), 1.47 (d, J=6.6 Hz, 3H).

### Example #21: N-(4-acetylphenyl)-2-[3-fluoro-5-(3-fluoroazetidin-1-yl)-9-oxo-4,8,12-triazatricyclo[9.4.0.0^{2,7}]pentadeca-1(11),2(7),3,5,12,14-hexaen-8-yl]acetamide

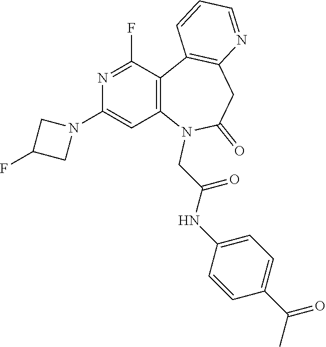 [SMILES: CC(=O)c1ccc(NC(=O)CN2C(=O)Cc3ncccc3-c3c2cc(N2CC(F)C2)nc3F)cc1]

### Step 1: Synthesis of N-(4-acetylphenyl)-2-(3,5-difluoro-9-oxo-4,8,12-triazatricyclo[9.4.0.0²⁷]pentadeca-1(11),2(7),3,5,12,14-hexaen-8-yl)acetamide

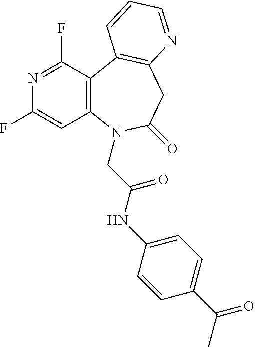 [SMILES: CC(=O)c1ccc(NC(=O)CN2C(=O)Cc3ncccc3-c3c2cc(F)nc3F)cc1]

To a solution of Intermediate L23 (776 mg, 3.08 mmol) and Intermediate C1 (748 mg, 3.08 mmol) in N,N-dimethylformamide (9 mL) was added at room temperature potassium carbonate (860 mg, 6.16 mmol). The resulting mixture was stirred at room temperature for 20 h. After completion, water and ethyl acetate were added and the reaction mixture was extracted three times with ethyl acetate. The combined organic layers were dried over MgSO₄, filtered, and concentrated under vacuum to afford the title compound (1.27 g, yield: 96%). LC-MS (Method A1_S) m/z: [M+H]⁺: 423.0; rt: 1.14 min; purity: 96%. ¹H NMR (400 MHz, DMSO-d₆) δ 10.63 (s, 1H), 8.64 (dd, J=4.9, 1.7 Hz, 1H), 8.19 (ddd, J=8.0, 4.7, 1.7 Hz, 1H), 7.97-7.90 (m, 2H), 7.74-7.66 (m, 2H), 7.54 (dd, J=8.0, 4.9 Hz, 1H), 7.34 (s, 1H), 4.69 (d, J=17.0 Hz, 1H), 4.64 (d, J=17.0 Hz, 1H), 4.06-3.98 (m, 1H), 3.75 (d, J=12.7 Hz, 1H), 2.53 (s, 3H).

### Step 2: Synthesis of N-(4-acetylphenyl)-2-[3-fluoro-5-(3-fluoroazetidin-1-yl)-9-oxo-4,8,12-triazatricyclo[9.4.0.0²⁷]pentadeca-1(11),2(7),3,5,12,14-hexaen-8-yl]acetamide Example #21

To a solution of N-(4-acetylphenyl)-2-(3,5-difluoro-9-oxo-4,8,12-triazatricyclo[9.4.0.0^{2,7}]pentadeca-1(11),2(7),3,5,12,14-hexaen-8-yl)acetamide (Example #21 step 1, 280 mg, 0.61 mmol) and 3-fluoroazetidine hydrochloride (85 mg, 0.76 mmol) in acetonitrile (5 mL) was added at room temperature N,N-diisopropylethylamine (0.3 mL, 2 mmol). The resulting mixture was stirred at room temperature for 14 days. After completion, water and DCM were added. The combined organic layers were separated, dried over MgSO₄, filtered, and concentrated under vacuum. The residue was purified by SFC chromatography (P4VP DCpak from Daicel, CO₂+MeOH 20%) to afford the title compound (51 mg, yield: 17%). LC-MS (Method A2′) m/z: [M+H]⁺: 478.1; rt: 3.89 min; purity: 100%. LC-MS (Method B2) m/z: [M+H]⁺: 478.0; rt: 4.07 min; purity: 100%. ¹H NMR (400 MHz, DMSO-d₆) δ 10.54 (s, 1H), 8.50 (dd, J=4.8, 1.6 Hz, 1H), 8.00 (ddd, J=7.9, 4.8, 1.6 Hz, 1H), 7.95-7.87 (m, 2H), 7.70-7.62 (m, 2H), 7.44 (dd, J=7.9, 4.8 Hz, 1H), 6.38 (s, 1H), 5.50 (dtd, J=60.4, 6.0, 3.1 Hz, 1H), 4.61 (d, J=16.6 Hz, 1H), 4.54 (d, J=16.6 Hz, 1H), 4.44-4.24 (m, 2H), 4.18-3.97 (m, 2H), 3.86 (d, J=12.4 Hz, 1H), 3.65 (d, J=12.4 Hz, 1H), 2.50 (s, 3H).

### Example #22: N-(4-acetylphenyl)-2-[5-(3,3-difluoroazetidin-1-yl)-3-fluoro-9-oxo-4,8,12-triazatricyclo[9.4.0.0^{2,7}]pentadeca-1(11),2(7),3,5,12,14-hexaen-8-yl]acetamide

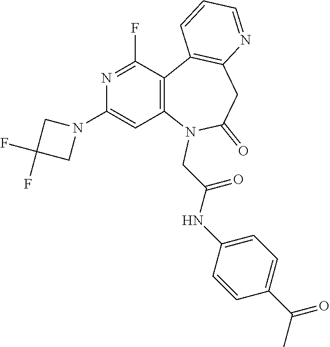 [SMILES: CC(=O)c1ccc(NC(=O)CN2C(=O)Cc3ncccc3-c3c2cc(N2CC(F)(F)C2)nc3F)cc1]

To a solution of N-(4-acetylphenyl)-2-(3,5-difluoro-9-oxo-4,8,12-triazatricyclo[9.4.0.0^{2,7}]pentadeca-1(11),2(7),3,5,12,14-hexaen-8-yl)acetamide (Example #21 step 1, 280 mg, 0.61 mmol) and 3,3-difluoroazetidine hydrochloride (95 mg, 0.73 mmol) in acetonitrile (5 mL) was added at room temperature N,N-diisopropylethylamine (0.3 mL, 2 mmol). The resulting mixture was stirred at room temperature for 14 days. After completion, water and DCM were added. The combined organic layers were separated, dried over MgSO₄, filtered, and concentrated under vacuum. The residue was purified by SFC chromatography (P4VP DCpak from Daicel, CO₂+MeOH 15%). The residue was washed with water and extracted with DCM to afford the title compound (77 mg, yield: 25%). LC-MS (Method A1_S) m/z: [M+H]⁺: 496.0; rt: 1.19 min; purity: 97%. ¹H NMR (400 MHz, DMSO-d₆) δ 10.56 (s, 1H), 8.55 (dd, J=4.9, 1.7 Hz, 1H), 8.05 (ddd, J=8.0, 4.7, 1.7 Hz, 1H), 7.97-7.90 (m, 2H), 7.74-7.64 (m, 2H), 7.48 (dd, J=8.0, 4.9 Hz, 1H), 6.54 (s, 1H), 4.71-4.40 (m, 6H), 3.90 (d, J=12.5 Hz, 1H), 3.77-3.66 (m, 1H), 2.53 (s, 3H).

### Example #23: enantiomer (10R) or (10S) of N-(4-acetylphenyl)-2-(3,5-difluoro-10-methyl-9-oxo-4,8,12-triazatricyclo[9.4.0.0^{2,7}]pentadeca-1(11),2(7),3,5,12,14-hexaen-8-yl)acetamide

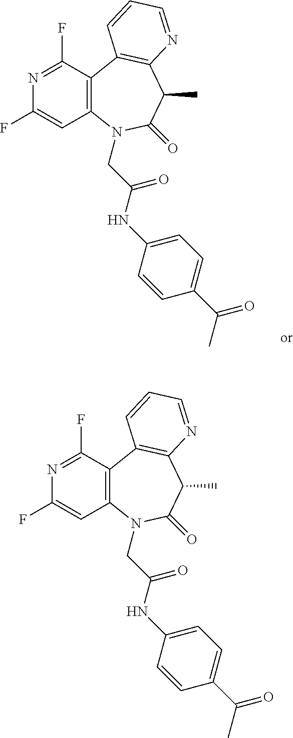 [SMILES: CC(=O)c1ccc(NC(=O)CN2C(=O)[C@H](C)c3ncccc3-c3c2cc(F)nc3F)cc1 ; CC(=O)c1ccc(NC(=O)CN2C(=O)[C@@H](C)c3ncccc3-c3c2cc(F)nc3F)cc1]

The racemate intermediate L6 was separated by Chiral HPLC (Reprosil Chiral NR-R from Dr Maisch, EtOH 50%−heptane 50%+DEA 0.1%) to give the title product (31.4 mg, yield: 27%). Chiral purity >99%; rt: 2.89 min (first eluting enantiomer). For information, second eluting enantiomer rt: 3.85 min. Both measured by HPLC (Reprosil Chiral NR-R from Dr Maisch, EtOH 50%−heptane 50%+DEA 0.1%). LC-MS (Method A2′) m/z: [M+H]⁺: 437.0; rt: 4.19 min; purity: 100%.

### Example #24: enantiomer (10R) or (10S) of N-(4-acetyl-3-fluoro-phenyl)-2-[(10)-3-fluoro-5,10-dimethyl-9-oxo-4,8,12-triazatricyclo[9.4.0.0^{2,7}]pentadeca-1(11),2(7),3,5,12,14-hexaen-8-yl]acetamide

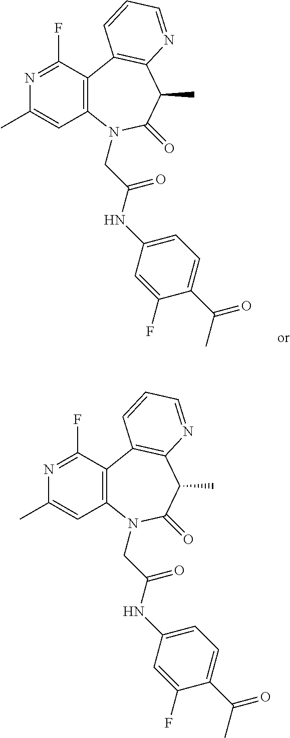 [SMILES: CC(=O)c1ccc(NC(=O)CN2C(=O)[C@H](C)c3ncccc3-c3c2cc(C)nc3F)cc1F ; CC(=O)c1ccc(NC(=O)CN2C(=O)[C@@H](C)c3ncccc3-c3c2cc(C)nc3F)cc1F]

### Step 1: Synthesis of N-(4-bromo-3-fluoro-phenyl)-2-(3-fluoro-5,10-dimethyl-9-oxo-4,8,12-triazatricyclo[9.4.0.0^{2,7}]pentadeca-1(11),2(7),3,5,12,14-hexaen-8-yl)acetamide

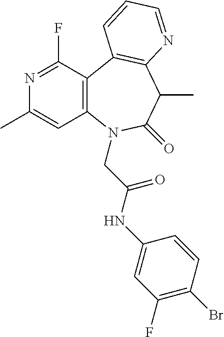 [SMILES: Cc1cc2c(c(F)n1)-c1cccnc1C(C)C(=O)N2CC(=O)Nc1ccc(Br)c(F)c1]

To a solution of Intermediate L1_6 (1.00 g, 3.89 mmol), N-(4-bromo-3-fluoro-phenyl)-2-chloro-acetamide (1.04 g, 3.89 mmol), N,N-dimethylformamide (15 mL) was added potassium carbonate (1.09 g, 7.77 mmol) and the reaction mixture was stirred at room temperature for 20 h. After completion, N-(4-bromo-3-fluoro-phenyl)-2-chloro-acetamide (1.04 g, 3.89 mmol) and potassium carbonate (1.09 g, 7.77 mmol) were added again and the reaction mixture was stirred and heated at 80° C. for 5 h. After completion, water and ethyl acetate were added and the reaction mixture was extracted three times with ethyl acetate. The combined organic layers were dried over MgSO₄, filtered, and concentrated under vacuum. The residue was triturated with 10 mL of iPr₂O and 2 mL of iPrOH to afford a solid (1.53 g, yield: 75%). LC-MS (Method A1_S) m/z: [M+H]⁺: 487.0/489.0; rt: 1.39 min; purity: 93%.

### Step 2: Synthesis of N-[4-(1-ethoxyvinyl)-3-fluoro-phenyl]-2-(3-fluoro-5,10-dimethyl-9-oxo-4,8,12-triazatricyclo[9.4.0.0^{2,7}]pentadeca-1(11),2(7),3,5,12,14-hexaen-8-yl)acetamide

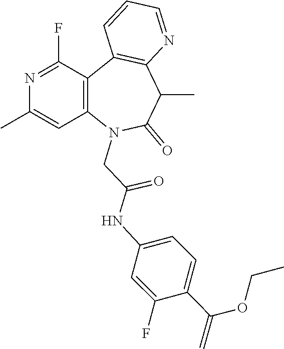 [SMILES: C=C(OCC)c1ccc(NC(=O)CN2C(=O)C(C)c3ncccc3-c3c2cc(C)nc3F)cc1F]

To a solution of N-(4-bromo-3-fluoro-phenyl)-2-[3,14-difluoro-5,10-dimethyl-9-oxo-4,8,12-triazatricyclo[9.4.0.0^{2,7}]pentadeca-1(11),2(7),3,5,12,14-hexaen-8-yl]acetamide (Example #24 step 1, 1.53 g, 3.15 mmol) in toluene (15 mL) were added tributyl(1-ethoxyvinyl)tin (1.31 mL, 3.76 mmol) and tetrakis(triphenylphosphine)palladium(0) (190 mg, 0.16 mmol) at room temperature. Then, the reaction mixture was heated at 110° C. for 20 h. After completion, water and ethyl acetate were added and the reaction mixture was filtered over a plug of celite. The resulting mixture was extracted with ethyl acetate. The combined organic layers were dried over MgSO₄, filtered, and concentrated under vacuum to afford the title compound (3.20 g, quantitative yield). LC-MS (Method B1_S) m/z: [M+H]⁺: 479.0; rt: 1.55 min; purity: 47%.

### Step 3: Synthesis of enantiomer (10R) or (10S) of N-(4-acetyl-3-fluoro-phenyl)-2-(3-fluoro-5,10-dimethyl-9-oxo-4,8,12-triazatricyclo[9.4.0.0^{2I}]pentadeca-1(11),2(7),3,5,12,14-hexaen-8-yl)acetamide Example #24

To a solution of N-[4-(1-ethoxyvinyl)-3-fluoro-phenyl]-2-(3-fluoro-5,10-dimethyl-9-oxo-4,8,12-triazatricyclo[9.4.0.0^{2,7}]pentadeca-1(11),2(7),3,5,12,14-hexaen-8-yl)acetamide (Example 24 step 2, 3.20 g, 6.02 mmol) in THE (100 mL) was added hydrochloric acid (1 mol/L in water, 20 mL) and the reaction mixture was stirred for 16 h at room temperature. After completion, the reaction mixture was quenched with a 1M solution of NaOH in water. The organic layers were extracted three times with DCM. The combined organic layers were dried over MgSO₄, filtered, and concentrated under vacuum. The residue was purified by column chromatography on silica gel (using a gradient of 0 to 100% EtOAc in heptane as eluant over 20 CV) to afford the corresponding racemate (1.20 g, yield: 85%). The racemate was separated by Chiral HPLC (Reprosil Chiral NR-R from Dr Maisch, EtOH 50%−heptane 50%+DEA 0.1%) to give the title product (355 mg, yield: 36%). Chiral purity >99%; rt: 2.66 min (first eluting enantiomer). For information, second eluting enantiomer rt: 3.42 min. Both measured by HPLC (Reprosil Chiral NR-R from Dr Maisch, EtOH 50%−heptane 50%+DEA 0.1%). LC-MS basic (Method B2) m/z: [M+H]⁺: 451.0; rt: 4.32 min; purity: 99%. LC-MS acid (Method A2′) m/z: [M+H]⁺: 451.0; rt: 4.21 min; purity: 100%. ¹H NMR (400 MHz, DMSO-d₆) δ 10.79 (s, 1H), 8.68 (dd, J=4.8, 1.7 Hz, 1H), 8.13 (ddd, J=8.0, 4.6, 2.0 Hz, 1H), 7.83 (t, J=8.7 Hz, 1H), 7.67 (dd, J=14.0, 2.0 Hz, 1H), 7.51 (dd, J=8.0, 4.6 Hz, 1H), 7.39-7.34 (m, 2H), 4.60 (s, 2H), 3.79 (q, J=6.6 Hz, 1H), 2.54 (d, J=4.4 Hz, 3H), 1.49 (d, J=6.6 Hz, 3H). CH₃ protons under DMSO signal.

### Example #25: N-(4-acetylphenyl)-2-[3-fluoro-5-(3-hydroxy-3-methyl-azetidin-1-yl)-10-methyl-9-oxo-4,8,12-triazatricyclo[9.4.0.0^{2,7}]pentadeca-1(11),2(7),3,5,12,14-hexaen-8-yl]acetamide

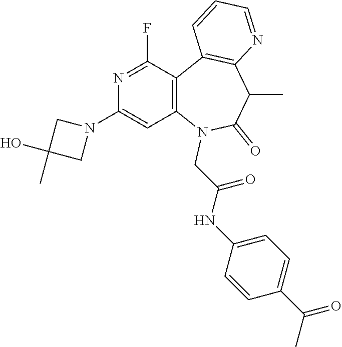 [SMILES: CC(=O)c1ccc(NC(=O)CN2C(=O)C(C)c3ncccc3-c3c2cc(N2CC(C)(O)C2)nc3F)cc1]

The title compound was prepared according to a similar procedure as for example #7 and starting from Intermediate L6 (102 mg, 0.23 mmol) and 3-hydroxy-3-methylazetidine hydrochloride (47 mg, 0.38 mmol). The residue was purified by SFC chromatography (P4VP DCpak from Daicel, CO₂+MeOH 25%) to afford the title compound (49 mg, yield: 42%). LC-MS (Method A2′) m/z: [M+H]⁺: 504.1; rt: 3.75 min; purity: 99.3%. LC-MS (Method B2) m/z: [M+H]⁺: 504.1; rt: 3.60 min; purity: 98.6%.

### Example #26: N-(4-acetylphenyl)-2-[3-fluoro-5-(3-fluoro-3-methyl-azetidin-1-yl)-10-methyl-9-oxo-4,8,12-triazatricyclo[9.4.0.0^{2,7}]pentadeca-1(11),2(7),3,5,12,14-hexaen-8-yl]acetamide

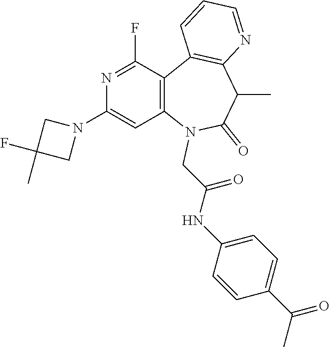 [SMILES: CC(=O)c1ccc(NC(=O)CN2C(=O)C(C)c3ncccc3-c3c2cc(N2CC(C)(F)C2)nc3F)cc1]

The title compound was prepared according to a similar procedure as for example #7 and starting from Intermediate L6 (102 mg, 0.23 mmol) and 3-fluoro-3-methylazetidine hydrochloride (45 mg, 0.36 mmol). The residue was purified by preparative HPLC (Purification Method P_B) followed by a SFC chromatography (GreenSep Nitro, CO₂+MeOH 30%) to afford the title compound (34 mg, yield: 29%). LC-MS (Method A2′) m/z: [M+H]⁺: 506.1; rt: 4.41 min; purity: 99.5%. LC-MS (Method B2) m/z: [M+H]⁺: 506.1; rt: 4.25 min; purity: 99.0%.

### Example #27: N-(4-acetylphenyl)-2-[3-fluoro-5-(3-hydroxy-3-methyl-azetidin-1-yl)-9-oxo-4,8,12-triazatricyclo[9.4.0.0^{2,7}]pentadeca-1(11),2(7),3,5,12,14-hexaen-8-yl]acetamide

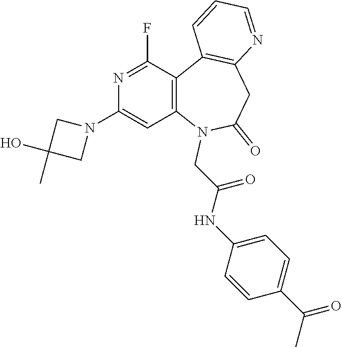 [SMILES: CC(=O)c1ccc(NC(=O)CN2C(=O)Cc3ncccc3-c3c2cc(N2CC(C)(O)C2)nc3F)cc1]

The title compound was prepared according to a similar procedure as for example #7 and starting from N-(4-acetylphenyl)-2-(3,5-difluoro-9-oxo-4,8,12-triazatricyclo[9.4.0.0^{2,7}]pentadeca-1(11),2(7),3,5,12,14-hexaen-8-yl)acetamide (Example #21 step 1, 102 mg, 0.24 mmol) and 3-fluoro-3-methylazetidine hydrochloride (45 mg, 0.36 mmol). The residue was purified by a SFC chromatography (P4VP DCpak from Daicel, CO₂+MeOH 25%) to afford the title compound (22 mg, yield: 19%). LC-MS (Method A2′) m/z: [M+H]⁺: 490.1; rt: 3.47 min; purity: 98.9%. LC-MS (Method B2) m/z: [M+H]⁺: 490.1; rt: 3.35 min; purity: 98.9%.

### Example #28: N-(4-acetylphenyl)-2-(14-fluoro-7,12-dimethyl-8-oxo-4-thia-5,9,13-triazatricyclo[8.4.0.0^{2,7}]tetradeca-1(10),2,5,11,13-pentaen-9-yl)acetamide

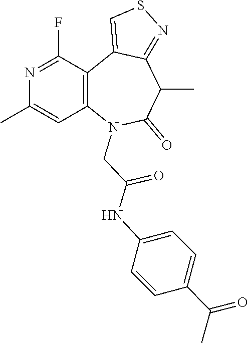 [SMILES: CC(=O)c1ccc(NC(=O)CN2C(=O)C(C)c3nscc3-c3c2cc(C)nc3F)cc1]

To a solution of intermediate L18 (38 mg, 0.14 mmol) in dry N,N-dimethylformamide (1 mL) was added Intermediate C1 (35 mg, 0.17 mmol) and potassium carbonate (40 mg, 0.29 mmol). The reaction mixture was stirred at room temperature for 18 h. After 18 h, the reaction mixture was diluted with EtOAc and water. The combined organic layers were washed with brine, dried over MgSO₄, filtered, and concentrated to dryness to afford a brown oil. The residue was purified by preparative HPLC (Purification Method P_B) to afford the title compound as a white solid (11 mg, yield: 17%) as a white solid. LC-MS (Method A4) m/z: no mass response; rt: 4.03 min; purity: 98%. LC-MS (Method B4) m/z: [M+H]⁺: 438; rt: 3.68 min; purity: 98%. ¹H NMR (500 MHz, DMSO-d₆) δ 10.60 (s, 1H), 9.40 (d, J=3.6 Hz, 1H), 7.96-7.90 (m, 2H), 7.75-7.67 (m, 2H), 7.36 (s, 1H), 4.62 (d, J=16.9 Hz, 1H), 4.53 (d, J=16.9 Hz, 1H), 3.89 (q, J=6.6 Hz, 1H), 2.48 (s, 3H), 1.52 (d, J=6.6 Hz, 3H). CH₃ protons under DMSO signal.

### Example #29: N-(4-acetylphenyl)-2-[3-fluoro-5-(1-hydroxycyclobutyl)-10-methyl-9-oxo-4,8,12-triazatricyclo[9.4.0.0^{2,7}]pentadeca-1(15),2,4,6,11,13-hexaen-8-yl]acetamide

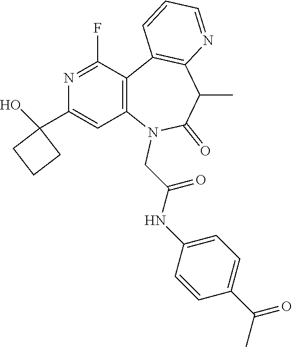 [SMILES: CC(=O)c1ccc(NC(=O)CN2C(=O)C(C)c3ncccc3-c3c2cc(C2(O)CCC2)nc3F)cc1]

To a solution of intermediate L19 (32 mg, 0.10 mmol) in dry N,N-dimethylformamide (0.5 mL) was added Intermediate C1 (28 mg, 0.12 mmol), potassium iodide (1.6 mg, 0.01 mmol) followed by potassium carbonate (34 mg, 0.24 mmol). The reaction mixture was stirred at rt for 18 h. The reaction mixture was diluted with EtOAc and water. The combined organic layers were washed with brine, dried over MgSO₄, filtered, and concentrated to dryness to afford a brown oil. The residue was purified by preparative HPLC (Purification Method P_B) to afford the title compound (3.8 mg, yield: 8%). LC-MS (Method A4) m/z: [M+H]⁺: 489; rt: 3.64 min; purity: 98.7%. LC-MS (Method B4) m/z: [M+H]⁺: 489; rt: 3.98 min; purity: 98.7%. ¹H NMR (500 MHz, DMSO) δ 10.61 (s, 1H), 8.69 (dd, J=4.8, 1.7 Hz, 1H), 8.20-8.16 (m, 1H), 7.96-7.90 (m, 2H), 7.73-7.67 (m, 2H), 7.60 (s, 1H), 7.52 (dd, J=8.0, 4.8 Hz, 1H), 5.99 (s, 1H), 4.63 (d, J=17.1 Hz, 1H), 4.50 (d, J=17.1 Hz, 1H), 3.81 (q, J=6.7 Hz, 1H), 2.60-2.55 (m, 2H), 2.53 (s, 3H), 2.28-2.19 (m, 2H), 2.02-1.82 (m, 2H), 1.50 (d, J=6.7 Hz, 3H).

### Example #30: N-(4-acetylphenyl)-2-[3,14-difluoro-5-(hydroxymethyl)-10-methyl-9-oxo-4,8,12-triazatricyclo[9.4.0.0^{2,7}]pentadeca-1(11),2,4,6,12,14-hexaen-8-yl]acetamide

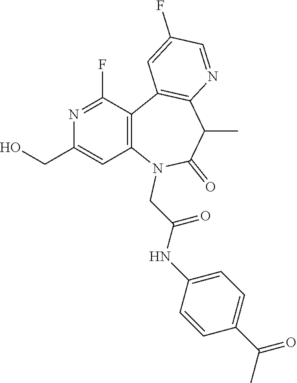 [SMILES: CC(=O)c1ccc(NC(=O)CN2C(=O)C(C)c3ncc(F)cc3-c3c2cc(CO)nc3F)cc1]

To a solution of intermediate L20 (19 mg, 0.06 mmol) in dry N,N-dimethylformamide (0.3 mL) was added Intermediate C1 (18 mg, 0.12 mmol), potassium iodide (1 mg, 0.01 mmol) followed by potassium carbonate (21 mg, 0.15 mmol). The reaction mixture was stirred at room temperature for 3 h. After completion, the reaction mixture was diluted with EtOAc and water. The combined organic layers were washed with brine, dried over MgSO₄, filtered, and concentrated to dryness to afford a brown oil. The residue was purified by preparative HPLC (Purification Method P_B) to afford the title compound (3.8 mg, yield: 12%). LC-MS (Method A4) m/z: [M+H]⁺: 467; rt: 3.65 min; purity: 92%. LC-MS (Method B4) m/z: [M+H]⁺: 467; rt: 3.35 min; purity: 93%. ¹H NMR (500 MHz, DMSO-d₆) δ 10.62 (s, 1H), 8.72 (d, J=2.7 Hz, 1H), 8.21-8.15 (m, 1H), 7.96-7.92 (m, 2H), 7.72-7.69 (m, 2H), 7.53 (s, 1H), 5.69 (t, J=5.8 Hz, 1H), 4.68-4.52 (m, 4H), 3.82 (q, J=6.6 Hz, 1H), 1.50 (d, J=6.6 Hz, 3H). CH₃ protons under DMSO signal.

### Example #31: enantiomer (10R) or (10S) of N-(4-acetylphenyl)-2-(5-cyclobutyl-3-fluoro-10-methyl-9-oxo-4,8,12-triazatricyclo[9.4.0.0^{2,7}]pentadeca-1(11),2(7),3,5,12,14-hexaen-8-yl)acetamide

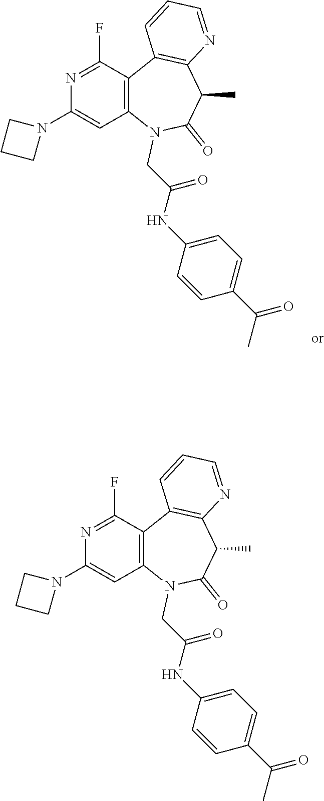 [SMILES: CC(=O)c1ccc(NC(=O)CN2C(=O)[C@H](C)c3ncccc3-c3c2cc(N2CCC2)nc3F)cc1 ; CC(=O)c1ccc(NC(=O)CN2C(=O)[C@@H](C)c3ncccc3-c3c2cc(N2CCC2)nc3F)cc1]

To a solution of Intermediate L21 (220 mg, 0.63 mmol) in dry N,N-dimethylformamide (10 mL) was added Intermediate C1 (188 mg, 0.89 mmol), potassium iodide (12 mg, 0.07 mmol) followed by potassium carbonate (206 mg, 1.48 mmol). The reaction mixture was stirred at room temperature for 20 h. After completion, the reaction mixture was quenched with saturated aqueous solution of NH₄Cl and extracted with ethyl acetate. The organic layers were washed with brine, dried over Na₂SO₄, filtered, and concentrated to afford a brown solid. The residue was purified by column chromatography on silica gel (using a gradient of 0 to 9% MeOH in DCM) followed by SFC chromatography (SFC-5-Diol from Kromasil, CO₂+MeOH 5 to 50%) to afford the corresponding racemate compound (189 mg, yield: 44%). The racemate was separated by Chiral SFC (Chiralpak IB from Daicel, CO₂+MeOH 30%) to give the title product (63.7 mg, yield: 21%). Chiral purity >99%; rt: 2.36 min (second eluting enantiomer). For information, first eluting enantiomer rt: 1.87 min). Both measured by HPLC (Chiralpak IB from Daicel, MeOH 100%+DEA 0.1%). LC-MS (Method A4) m/z: [M+H]⁺: 473.0; rt: 5.26 min; purity: 99.2%. LC-MS (Method B4) m/z: [M+H]⁺: 473.0; rt: 4.58 min; purity: 98.8%. ¹H NMR (500 MHz, DMSO-d₆) δ 10.58 (s, 1H), 8.67 (dd, J=4.8, 1.7 Hz, 1H), 8.15 (ddd, J=7.9, 4.8, 1.7 Hz, 1H), 7.96-7.90 (m, 2H), 7.72-7.65 (m, 2H), 7.50 (dd, J=7.9, 4.8 Hz, 1H), 7.33 (s, 1H), 4.62 (s, 2H), 3.79 (q, J=6.6 Hz, 1H), 3.68 (p, J=8.6 Hz, 1H), 2.52 (s, 3H), 2.36-2.23 (m, 4H), 2.07-1.94 (m, 1H), 1.91-1.80 (m, 1H), 1.48 (d, J=6.6 Hz, 3H). ¹⁹F NMR (471 MHz, DMSO-d₆) 5-70.10 (d, J=4.8 Hz).

### Example #32: enantiomer (10R) or (10S) of N-(4-acetylphenyl)-2-(14-fluoro-5-methoxy-10-methyl-9-oxo-4,8,12-triazatricyclo[9.4.0.0^{2,7}]pentadeca-1(11),2(7),3,5,12,14-hexaen-8-yl)acetamide

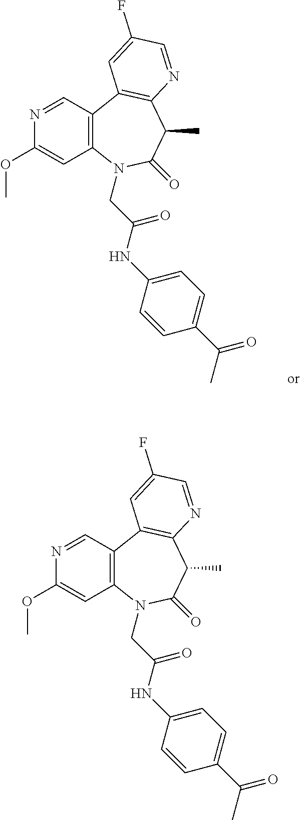 [SMILES: COc1cc2c(cn1)-c1cc(F)cnc1[C@@H](C)C(=O)N2CC(=O)Nc1ccc(C(C)=O)cc1 ; COc1cc2c(cn1)-c1cc(F)cnc1[C@H](C)C(=O)N2CC(=O)Nc1ccc(C(C)=O)cc1]

To a solution of Intermediate L24 (100 mg, 0.37 mmol) and Intermediate C1 (107 mg, 0.44 mmol) in N,N-dimethylformamide (1.8 mL) were added at room temperature potassium carbonate (128 mg, 0.92 mmol) and potassium iodide (6 mg, 0.04 mmol). The resulting mixture was stirred at room temperature for 18 h. After completion, water and ethyl acetate were added and the reaction mixture was extracted three times with ethyl acetate. The combined organic layers were dried over MgSO₄, filtered, and concentrated undervacuum to afford a brown solid. The residue was triturated with a mixture of water and acetonitrile (3/7) to afford racemic N-(4-acetylphenyl)-2-(14-fluoro-5-methoxy-10-methyl-9-oxo-4,8,12-triazatricyclo[9.4.0.02,7]pentadeca-1 (11),2(7),3,5,12,14-hexaen-8-yl)acetamide (107 mg, yield: 63%) as a white solid. The racemate was separated by Chiral HPLC (Reprosil Chiral NR-R from Dr Maisch, EtOH 50%−heptane 50%+DEA 0.1%) to give the title product (34.4 mg, yield: 20%). Chiral purity >99%; rt: 3.42 min (first eluting enantiomer). For information, second eluting enantiomer rt: 4.40 min. Both measured by HPLC (Reprosil Chiral NR-R from Dr Maisch, EtOH 50%−heptane 50%+DEA 0.1%). LC-MS (Method A4) m/z: [M+H]⁺: 449; rt: 4.25 min; purity: 99.6%. LC-MS (Method B4) m/z: [M+H]⁺: 449; rt: 4.05 min; purity: 96.29%. ¹H NMR (500 MHz, DMSO-d₆) δ 10.58 (s, 1H), 8.66 (d, J=2.8 Hz, 1H), 8.53 (s, 1H), 8.15 (dd, J=9.5, 2.8 Hz, 1H), 7.96-7.90 (m, 2H), 7.71-7.65 (m, 2H), 6.93 (s, 1H), 4.66 (d, J=16.9 Hz, 1H), 4.60 (d, J=16.9 Hz, 1H), 3.95 (s, 3H), 3.72 (q, J=6.7 Hz, 1H), 2.53 (s, 3H), 1.48 (d, J=6.7 Hz, 3H). ¹⁹F NMR (471 MHz, DMSO-d₆) δ −129.80 (d, J=9.5 Hz).

### Example #33: enantiomer (10R) or (10S) of N-(4-acetyl-3-hydroxy-phenyl)-2-(3,14-difluoro-5,10-dimethyl-9-oxo-4,8,12-triazatricyclo[9.4.0.0^{2,7}]pentadeca-1(11),2(7),3,5,12,14-hexaen-8-yl)acetamide

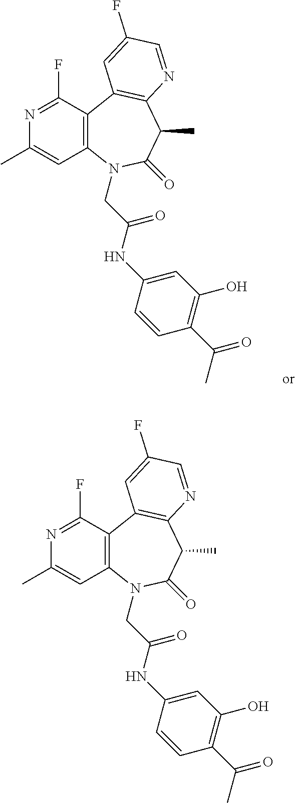 [SMILES: CC(=O)c1ccc(NC(=O)CN2C(=O)[C@H](C)c3ncc(F)cc3-c3c2cc(C)nc3F)cc1O ; CC(=O)c1ccc(NC(=O)CN2C(=O)[C@@H](C)c3ncc(F)cc3-c3c2cc(C)nc3F)cc1O]

To a mixture of 2-(3,14-difluoro-5,10-dimethyl-9-oxo-4,8,12-triazatricyclo[9.4.0.0^{2,7}]pentadeca-1(11),2(7),3,5,12,14-hexaen-8-yl)acetamide Intermediate L16 (100 mg, 0.30 mmol) in dry 1,4-dioxane (3 mL) was added at room temperature 1-(4-bromo-2-hydroxyphenyl)ethanone (82 mg, 0.36 mmol). The resulting mixture was flushed with N₂ before addition of XPhos Pd G3 (27 mg, 0.03 mmol), XPhos (15 mg, 0.03 mmol) and cesium carbonate (294 mg, 0.90 mmol). The resulting mixture was stirred at 100° C. for 2 h. After completion, the reaction was diluted with EtOAc (10 mL), filtered over a pad of celite and rinsed with EtOAc. The filtrate was concentrated under vacuum to afford racemic N-(4-acetyl-3-hydroxy-phenyl)-2-(3,14-difluoro-5,10-dimethyl-9-oxo-4,8,12-triazatricyclo[9.4.0.0^{2,7}]pentadeca-1(11),2(7),3,5,12,14-hexaen-8-yl)acetamide After purification by reverse phase chromatography (basic elution), the racemate was separated by Chiral HPLC (Chiralpak IG from Daicel, EtOH 50%−Heptane 50%) to afford the title compound as a white solid 18 mg, yield: 13%). Chiral purity: 98.3%; rt: 4.97 min (second eluting enantiomer). For information, the first eluting enantiomer rt: 2.69 min. Both measured by HPLC (Chiralpak IG from Daicel, EtOH 50%−Heptane 50%−DEA 0.1%). LC-MS (Method A2) m/z: [M+H]⁺: 466.9; rt: 4.53 min; purity: 99.6%. LC-MS (Method B2) m/z: [M+H]⁺: 467.2; rt: 4.25 min; purity: 99.5%. ¹H NMR (400 MHz, DMSO) δ 12.28 (s, 1H), 10.57 (s, 1H), 8.71 (d, J=2.7 Hz, 1H), 8.21-8.09 (m, 1H), 7.86 (d, J=8.7 Hz, 1H), 7.38 (s, 1H), 7.31 (s, 1H), 7.04 (d, J=8.7 Hz, 1H), 4.59 (s, 2H), 3.81 (q, J=6.6 Hz, 1H), 2.57 (s, 3H), 1.48 (d, J=6.6 Hz, 3H). CH₃ protons under DMSO signal. ¹⁹F NMR (376 MHz, DMSO) δ −70.59 (d, J=3.6 Hz), −130.19 (d, J=9.6 Hz).

### Example #34: N-(4-acetylphenyl)-2-(3,14-difluoro-5,10-dimethyl-9-oxo-4,8,13-triazatricyclo[9.4.0.0^{2,7}]pentadeca-1(11),2(7),3,5,12,14-hexaen-8-yl)acetamide

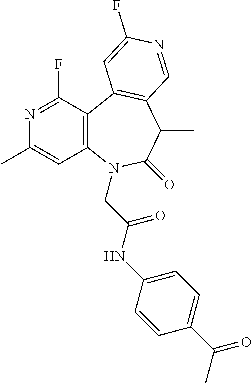 [SMILES: CC(=O)c1ccc(NC(=O)CN2C(=O)C(C)c3cnc(F)cc3-c3c2cc(C)nc3F)cc1]

Example #34 was prepared according to a similar procedure as for example #3 starting from Intermediate L13 (1.7 mg, 0.006 mmol) and Intermediate C1 (1.4 mg, 0.007 mmol). After completion, the reaction mixture was purified by preparative HPLC (Purification Method P_B) to afford the title compound as a white solid (1.68 mg, yield: 59%). LC-MS (Method A8) m/z: [M+H]⁺: 451.1; rt: 3.47 min; purity: 98%. LC-MS (Method B8) m/z: [M+H]⁺: 451.1; rt: 3.14 min; purity: 99%. ¹H NMR (400 MHz, DMSO-d₆) δ 10.56 (s, 1H), 8.28 (s, 1H), 7.93 (d, J=8.8 Hz, 2H), 7.68 (d, J=8.8 Hz, 2H), 7.55 (dd, J=4.3, 1.7 Hz, 1H), 7.39 (s, 1H), 4.60 (s, 2H), 3.72 (q, J=6.8 Hz, 1H), 2.52 (s, 3H), 1.54 (d, J=6.8 Hz, 3H). CH₃ protons under DMSO signal. ¹⁹F NMR (376 MHz, DMSO-d₆) δ −70.02 (d, J=4.3 Hz), −71.65.

### Example #35: enantiomer (5R) or (5S) of N-(4-acetylphenyl)-2-[11-fluoro-9-(3-fluoroazetidin-1-yl)-5-methyl-6-oxo-5H-pyrido[2,3-d][1]benzazepin-7-yl]acetamide

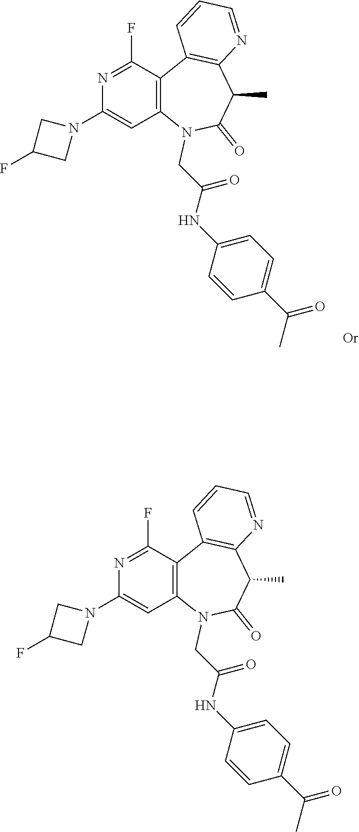 [SMILES: CC(=O)c1ccc(NC(=O)CN2C(=O)[C@H](C)c3ncccc3-c3c2cc(N2CC(F)C2)nc3F)cc1 ; CC(=O)c1ccc(NC(=O)CN2C(=O)[C@@H](C)c3ncccc3-c3c2cc(N2CC(F)C2)nc3F)cc1]

To a solution of Intermediate L25 (65 mg, 0.21 mmol) in DMF (0.7 mL) were added at room temperature potassium carbonate (60 mg, 0.43 mmol) and Intermediate C1 (51 mg, 0.21 mmol). The resulting mixture was stirred at room temperature for 2 days. After completion, water and ethyl acetate were added and the reaction mixture was extracted three times with ethyl acetate. The combined organic layers were dried over MgSO₄, filtered, and concentrated under vacuum. The residue was purified by preparative HPLC (Purification Method P_B) to afford the corresponding racemate as a white solid (56 mg, yield: 54%). The racemate was separated by Chiral HPLC (Whelk O-1 (R,R) from Regis Technology, EtOH 100%) to give the title product (17.6 mg, yield: 17%). Chiral purity >99%; rt: 2.38 min (first eluting enantiomer). For information, second eluting enantiomer rt: 2.92 min). Both measured by HPLC(Whelk O-1 (R,R) from Regis Technology, EtOH 100%+DEA 0.1%). LC-MS (Method A2′) m/z: [M+H]⁺: 491.0; rt: 4.36 min; purity: 97.6%. LC-MS (Method B2) m/z: [M+H]⁺: 491.0; rt: 4.53 min; purity: 99.0%.

### Example #36: N-(4-acetylphenyl)-2-(14-chloro-4-fluoro-7,12-dimethyl-8-oxo-5,6,9,13-tetrazatricyclo[8.4.0.0^{2,7}]tetradeca-1(14),2,4,10,12-pentaen-9-yl)acetamide

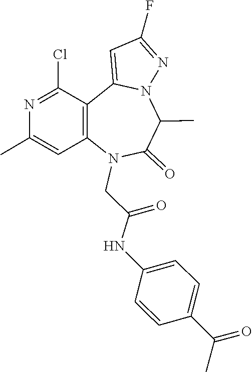 [SMILES: CC(=O)c1ccc(NC(=O)CN2C(=O)C(C)n3nc(F)cc3-c3c2cc(C)nc3Cl)cc1]

Example #36 was prepared according to a similar procedure as for example #3 starting from Intermediate L26 (54 mg, 0.19 mmol) and Intermediate C1 (45 mg, 0.20 mmol). After completion, the reaction mixture was purified by column chromatography on silica gel (using a gradient of 0-60% EtOAc/iso-Hexane as eluent) to afford the title compound (62 mg, yield: 69%) as a white solid. LC-MS (Method B5) m/z [M+H]⁺: 456.1/458.1; rt: 1.39 min; purity: 98%. 1H NMR (400 MHz, DMSO-d₆) δ 10.60 (s, 1H), 7.99-7.89 (m, 2H), 7.74-7.65 (m, 2H), 7.48 (s, 1H), 6.64 (d, J=5.8 Hz, 1H), 5.05-4.96 (m, 1H), 4.69-4.55 (m, 2H), 2.53 (s, 3H), 2.52 (s, 3H), 1.60 (d, J=6.7 Hz, 3H). 19F NMR (376 MHz, DMSO-d₆) δ −131.08.

### V. Biological Assays

The effect of compounds of Formula (I) on the system Xc⁻ functionality was examined in two independent cell assays: (i) measurement of cystine-induced glutamate release; and (ii) measurement of [¹⁴C]L-Cystine uptake.

As described above, System Xc⁻, also known as the cystine/glutamate antiporter, is an amino acid transporter that mediates the extrusion of intracellular L-glutamate outside the cell and the uptake of extracellular L-cystine into the cell.

By measuring respectively, the concentration of glutamate released outside the cell and the L-cystine uptaken in the cell, with increasing concentrations of compounds of formula (I) according to the present invention, the efficacy of said compounds to inhibit the System Xc⁻ function can be evaluated.

The efficacy of the compounds to inhibit system Xc⁻ is represented by measuring the IC₅₀ which corresponds to the concentration of compound necessary to inhibit 50% of the signals from the two control groups; DMSO 1% and Erastin 50 μM in the respective assays. pIC₅₀ values correspond to −log of the IC₅₀ in Molar.

The lower the value of the IC₅₀ (the higher the value of the pIC₅₀ is), the less compound is needed to perform the same amount of inhibition and therefore the higher is the inhibition potency.

Generally, inhibitors of system Xc⁻ function will display values of IC₅₀ of 500 nM or lower respectively in each of the cystine-induced glutamate release and [¹⁴C]L-Cystine uptake assays.

When tested in each of the cystine-induced glutamate release and [¹⁴C]L-Cystine uptake assays, compounds of formula (I) according to the present invention display values of pIC₅₀ generally greater than or equal to about 6.3, suitably greater than about 7.0, ideally greater than about 7.4, appositely greater than about 7.8.

### V.1. Cystine-Induced Glutamate Release Assay

Measurement of cystine-induced glutamate release indicates levels of glutamate (outside of the cell) that are dependent on system Xc⁻ antiporter function. Upon the addition of L-Cystine to H4 cells, intracellular glutamate is counter-transported and exported in the supernatant.

In this assay, the medium is devoid of sodium to prevent the transport of glutamate by sodium-dependent excitatory amino acid transporters (EAATs). H4 cells were plated on 384-well culture plate at 1×10⁴ cells/well in Dulbecco's Modified Eagle Medium (DMEM) with L-glutamine, penicillin/streptomycin and 10% fetal bovine serum (all from ThermoFisher Scientific) and incubated in a humidified CO₂ incubator at 37° C. After 2 days, cells were washed 4 times with prewarmed sodium-free HEPES buffer (contents in mM: HEPES 10, KCl 5.4, CaCl₂ 2.5, MgCl₂ 1, KH₂PO₄ 0.4, D-Glucose 5, Choline chloride 140), pH 7.4. Glutamate release was induced by addition of 50 μM of L-Cystine (Sigma-Aldrich), together with a test compound of Formula (I) at 1% final DMSO concentration. After 2 hours of incubation in the culture incubator, supernatants were transferred to a low-binding polypropylene 384-well plate to determine L-glutamate concentration using Amplex™ Red Glutamic Acid Assay Kit (ThermoFisher Scientific). Briefly, 10 μL of Amplex™ Red working solution was added to 10 μL of the supernatant, then the mixture was incubated for 30 min at 37° C. Fluorescence intensity was measured by EnVision microplate reader (PerkinElmer).

When tested, compounds of formula (I) according to the present invention have displayed a PIC₅₀ of about 6.3 or greater in the cystine-induced glutamate release assay.

### V.2. [¹⁴C]L-Cystine Uptake Assay

In this assay, the medium is devoid of sodium to prevent the transport of glutamate by sodium-dependent excitatory amino acid transporters (EAATs). H4 cells were plated on a 96-well CytoStar-T scintillating microplate (PerkinElmer) at 5×10⁴ cells/well in the medium described above. After 1 day, cells were washed one time with the sodium-free HEPES buffer and preincubated with test compounds of Formula (I) (1% final DMSO) for 15 min. Uptake was initiated by adding 0.5 μM L-[1,2, 1′,2′-¹⁴C] Cystine (0.02 mCi/mL, PerkinElmer, Waltham, US) and 4.5 μM L-Cystine (Sigma-Aldrich). After 1 hour at 37° C., plates were counted in a MicroBeta² microplate counter (PerkinElmer).

Table I exhibits the ranges of PIC₅₀ of the compounds of formula (I) according to the present invention when tested in the Cystine-induced glutamate release assay and/or [14C]L-Cystine uptake assay.

Category A: about 6.3<pIC₅₀≤about 7.00; Category B: about 7.00<pIC₅₀≤about 7.40; Category C: about 7.40<pIC₅₀≤about 7.80; Category D: pIC₅₀>about 7.80. n.t.: not tested.

| | Cystine- | [¹⁴C] L- |
|---|---|---|
| | induced | Cystine |
| | glutamate | uptake |
| Example | release assay | assay |
| #1 | C | C |
| #2 | A | A |
| #3 | C | C |
| #4 | B | B |
| #5 | D | D |
| #6 | D | D |
| #7 | B | D |
| #8 | D | D |
| #9 | A | B |
| #10 | A | B |
| #11 | C | C |
| #12 | B | B |
| #13 | C | D |
| #14 | B | C |
| #15 | B | B |
| #16 | C | D |
| #17 | B | D |
| #18 | B | B |
| #19 | C | D |
| #20 | B | C |
| #21 | C | D |
| #22 | B | C |
| #23 | B | B |
| #24 | B | C |
| #25 | B | C |
| #26 | A | C |
| #27 | B | C |
| #28 | B | A |
| #29 | B | B |
| #30 | A | A |
| #31 | C | C |
| #32 | A | C |
| #33 | D | D |
| #34 | B | B |
| #35 | C | D |
| #36 | n.t. | n.t. |

As shown in this Table I, compounds of formula (I) according to the present invention are potent inhibitors of the System Xc⁻ function.

1. A compound of formula (I) or a pharmaceutically acceptable salt thereof, 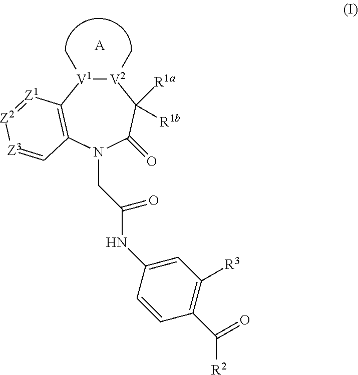
Wherein A together with the points of attachment V¹ and V² to the remainder of the molecule, represents an optionally substituted aryl or heteroaryl selected from the groups represented by A¹, A² and A³ 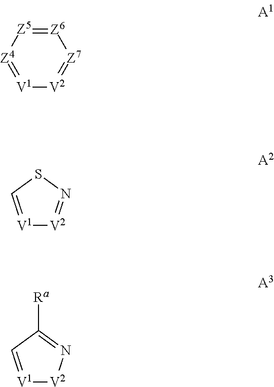
Wherein
V¹ represents C;
V² represents C or N;
Z⁴ represents N or C—R⁷;
Z⁵ represents N or C—R⁸;
Z⁶ represents N or C—R⁹;
Z⁷ represents N or C—R¹⁰;
R^{a} represents halogen;
R⁷, R⁸, R⁹, and R¹⁰ represent independently hydrogen or halogen; or C₁₋₄ alkyl or C₁₋₄ alkoxy, either of which groups may be optionally substituted by one or more substituents; and
Z¹ represents N or C—R⁴;
Z² represents N or C—R⁵;
Z³ represents N or C—R⁶;
Z⁴ represents N or C—R⁷;
R^{1a} and R^{1b} represent independently hydrogen; or C₁₋₄ alkyl, which group may be optionally substituted with one or more substituents;
R² represents C₁₋₄ alkyl or C₃₋₇ cycloalkyl, either of which groups may be optionally substituted by one or more substituents;
R³ represents hydrogen, halogen or hydroxyl; or C₁₋₄ alkyl, which group may be optionally substituted by one or more substituents; or
R² and R³ together with the phenyl group to which they are attached form an heteroaryl which group is optionally substituted with one or more substituents; and
R⁴ and R⁵ represent independently hydrogen, halogen or cyano; or C₁₋₄ alkyl or C₁₋₄ alkoxy, either of which groups may be optionally substituted by one or more substituents; and
R⁶ represents hydrogen, halogen or cyano; or C₁₋₄ alkyl, C₁₋₄ alkoxy or C₃₋₇ heterocycloalkyl, or C₃₋₇ cycloalkyl, any of which groups may be optionally substituted by one or more substitutents.

2. A compound of Formula (I) according to claim 1 wherein A represents A¹ and V¹ and V² independently represent C.

3. A compound of Formula (I) according to claim 1 wherein A represents A² and V¹ and V² independently represent C.

4. A compound of Formula (I) according to claim 1 wherein A represents A³, V¹ represents C, V² represents N and R^{a} represents fluoro.

5. A compound of Formula (I) according to claim 1 wherein Z² represents N, Z¹ represents C—R⁴ and Z³ represents C—R⁶.

6. A compound of Formula (I) according to claim 1 wherein Z⁷ represents N, Z⁴ represents C—R⁷, Z⁵ represents C—R⁸, Z⁶ represents C—R⁹.

7. A compound of Formula (I) according to claim 1 wherein R^{1a}, R^{1b}, R², R³, R⁴, R⁵, R⁶, R⁷, R⁸, R⁹ and R¹⁰ are optionally substituted by hydroxyl, halogen, C₁₋₄ alkyl, or C₁₋₄ alkoxy.

8. A compound of Formula (I) according to claim 1 wherein R^{1a}, Rib, R², R³, R⁴, R⁵, R⁷, R⁸, R⁹, and R¹⁰ are unsubstituted.

9. A compound of Formula (I) according to claim 1 wherein
R^{1a} represents C₁₋₄ alkyl and R^{1b} represents hydrogen;
R² represents C₁₋₄ alkyl or C₃₋₇ cycloalkyl;
R³ represents hydrogen;
R⁴, R⁷, R⁸, and R⁹ represent independently hydrogen, halogen or C₁₋₄ alkoxy; and
R⁶ represents C₁₋₄ alkyl, C₁₋₄ alkoxy or optionally substituted C₃₋₇ cycloalkyl or C₃₋₇ heterocycloalkyl.

10. A compound of Formula (I) according to claim 1, represented by formula (IA), 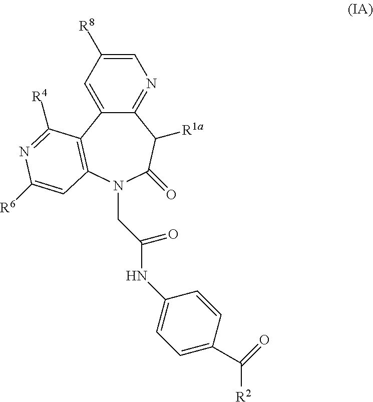
wherein
R^{1a} represents C₁₋₄ alkyl;
R² represents C₁₋₄ alkyl or C₃₋₇ cycloalkyl;
R⁴ and R⁸ represent independently hydrogen, halogen, or C₁₋₄ alkoxy; and
R⁶ represents C₁₋₄ alkyl, C₁₋₄ alkoxy or C₃₋₇ heterocycloalkyl.

11. A compound of formula (I) according to claim 1 wherein R^{1a} is C₁₋₄ alkyl.

12. A compound of formula (I) according to claim 1, wherein R² is C₁₋₄ alkyl.

13. A compound of formula (I) according to claim 1 wherein R⁴ is a halogen.

14. A compound of formula (I) according to claim 1 wherein R⁸ is a halogen.

15. A compound of formula (I) according to claim 1 selected from the group consisting of
N-(4-acetylphenyl)-2-[3-fluoro-5,10-dimethyl-9-oxo-4,8,12-triazatricyclo[9.4.0.0^{2,7}]pentadeca-1(11),2,4,6,12,14-hexaen-8-yl]acetamide;
N-[4-(cyclopropanecarbonyl)phenyl]-2-[3-fluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[3,14-difluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[14-fluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[(10R)-3-chloro-14-fluoro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[3-chloro-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[5-(3,3-difluoroazetidin-1-yl)-3-fluoro-10-methyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[3-fluoro-5-(3-fluoroazetidin-1-yl)-10-methyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[3-chloro-5-(2,2-difluoro-5-azaspiro[2.3]hexan-5-yl)-9-oxo-4,8,12-triazatricyclo[9.4.0.0^{2,7}]pentadeca-1(11),2,4,6,12,14-hexaen-8-yl]acetamide; and
N-(4-acetylphenyl)-2-(1-fluoro-3-methyl-6-oxo-7H-pyrido[4,3-d][3]benzazepin-5-yl)acetamide, N-(4-acetylphenyl)-2-[3-fluoro-5-methoxy-10-methyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[14-methoxy-5,10-dimethyl-9-oxo-4,8,12-triazatricyclo[9.4.0.0^{2,7}]pentadeca-1(11),2,4,6,12,14-hexaen-8-yl]acetamide;
N-(4-acetylphenyl)-2-[3-methoxy-5,10-dimethyl-9-oxo-4,8,12-triazatricyclo[9.4.0.0^{2,7}]pentadeca-1(15),2(7),3,5,11,13-hexaen-8-yl]acetamide;
N-(4-acetylphenyl)-2-(3-methoxy-10-methyl-9-oxo-4,8,12-triazatricyclo[9.4.0.0^{2,7}]pentadeca-1(11),2(7),3,5,12,14-hexaen-8-yl)acetamide;
2-[3-fluoro-5,10-dimethyl-9-oxo-4,8,12-triazatricyclo[9.4.0.0^{2,7}]pentadeca-1(11),2,4,6,12,14-hexaen-8-yl]-N-(4-propanoylphenyl)acetamide;
N-(4-acetyl-3-hydroxy-phenyl)-2-[3-fluoro-5,10-dimethyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-(3,14-difluoro-5-methoxy-10-methyl-9-oxo-4,8,12-triazatricyclo[9.4.0.0^{2,7}]pentadeca-1(11),2(7),3,5,12,14-hexaen-8-yl)acetamide;
2-[3,14-difluoro-5,10-dimethyl-9-oxo-4,8,12-triazatricyclo[9.4.0.0^{2,7}]pentadeca-1(11),2(7),3,5,12,14-hexaen-8-yl]-N-(3-oxobenzofuran-6-yl)acetamide;
N-(4-acetylphenyl)-2-(10-cyano-2-fluoro-5,9-dimethyl-6-oxo-5H-pyrido[2,3-d][1]benzazepin-7-yl)acetamide;
N-(4-acetylphenyl)-2-[(10)-3-fluoro-14-methoxy-5,10-dimethyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[3-fluoro-5-(3-fluoroazetidin-1-yl)-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[5-(3,3-difluoroazetidin-1-yl)-3-fluoro-9-oxo-4,8,12-triazatricyclo[9.4.0.0^{2,7}]pentadeca-1(11),2(7),3,5,12,14-hexaen-8-yl]acetamide;
N-(4-acetylphenyl)-2-(3,5-difluoro-10-methyl-9-oxo-4,8,12-triazatricyclo[9.4.0.0^{2,7}]pentadeca-1(11),2(7),3,5,12,14-hexaen-8-yl)acetamide;
N-(4-acetyl-3-fluoro-phenyl)-2-[(10)-3-fluoro-5,10-dimethyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[3-fluoro-5-(3-hydroxy-3-methyl-azetidin-1-yl)-10-methyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[3-fluoro-5-(3-fluoro-3-methyl-azetidin-1-yl)-10-methyl-9-oxo-4,8,12-triazatricyclo[9.4.0.0^{2,7}]pentadeca-1(11),2(7),3,5,12,14-hexaen-8-yl]acetamide;
N-(4-acetylphenyl)-2-[3-fluoro-5-(3-hydroxy-3-methyl-azetidin-1-yl)-9-oxo-4,8,12-triazatricyclo[9.4.0.0^{2,7}]pentadeca-1(11),2(7),3,5,12,14-hexaen-8-yl]acetamide;
N-(4-acetylphenyl)-2-(14-fluoro-7,12-dimethyl-8-oxo-4-thia-5,9,13-triazatricyclo[8.4.0.0^{2,6}]tetradeca-1(10),2,5,11,13-pentaen-9-yl)acetamide;
N-(4-acetylphenyl)-2-[3-fluoro-5-(1-hydroxycyclobutyl)-10-methyl-9-oxo-4,8,12-triazatricyclo[9.4.0.0^{2,7}]pentadeca-1(15),2,4,6,11,13-hexaen-8-yl]acetamide;
N-(4-acetylphenyl)-2-[3,14-difluoro-5-(hydroxymethyl)-10-methyl-9-oxo-4,8,12-triazatricyclo[9.4.0.0^{2,7}]pentadeca-1(11),2,4,6,12,14-hexaen-8-yl]acetamide;
N-(4-acetylphenyl)-2-(5-cyclobutyl-3-fluoro-10-methyl-9-oxo-4,8,12-triazatricyclo[9.4.0.0^{2,7}]pentadeca-1(11),2(7),3,5,12,14-hexaen-8-yl)acetamide;
N-(4-acetylphenyl)-2-(14-fluoro-5-methoxy-10-methyl-9-oxo-4,8,12-triazatricyclo[9.4.0.0^{2,7}]pentadeca-1(11),2(7),3,5,12,14-hexaen-8-yl)acetamide;
N-(4-acetyl-3-hydroxy-phenyl)-2-(3,14-difluoro-5,10-dimethyl-9-oxo-4,8,12-triazatricyclo[9.4.0.0^{2,7}]pentadeca-1(11),2(7),3,5,12,14-hexaen-8-yl)acetamide;
N-(4-acetylphenyl)-2-(3,14-difluoro-5,10-dimethyl-9-oxo-4,8,13-triazatricyclo[9.4.0.0^{2,7}]pentadeca-1(11),2(7),3,5,12,14-hexaen-8-yl)acetamide;
N-(4-acetylphenyl)-2-[11-fluoro-9-(3-fluoroazetidin-1-yl)-5-methyl-6-oxo-5H-pyrido[2,3-d][1]benzazepin-7-yl]acetamide;
N-(4-acetylphenyl)-2-(14-chloro-4-fluoro-7,12-dimethyl-8-oxo-5,6,9,13-tetrazatricyclo[8.4.0.0^{2,6}]tetradeca-1(14),2,4,10,12-pentaen-9-yl)acetamide;
and enantiomers thereof.

16. (canceled)

17. A method for the treatment of cancers or epilepsy syndromes where System Xc- plays a role, or cancer treatment resistance, which comprises administering to a patient in need of such a treatment an effective amount of a compound of Formula (I) according to claim 1.

18. (canceled)

19. A compound of formula (I) according to claim 10 wherein R^{1a} is C₁₋₄ alkyl.

20. A compound of formula (I) according to claim 10 wherein R² is C₁₋₄ alkyl.

21. A compound of formula (I) according to claim 10 wherein R⁴ is a halogen.

22. A compound of formula (I) according to claim 10 wherein R⁸ is a halogen.
